(19) United States
(12) Patent Application Publication
(10) Pub. No.: US 2026/0132164 A1
(43) Pub. Date: May 14, 2026

(54) PNU ANTHRACYCLINE DERIVATIVES AND METHODS OF USE THEREOF

(71) Applicant: Merck Sharp & Dohme LLC, Rahway, NJ (US)

(72) Inventors: Sijie Chen, Millbrae, CA (US); John A. Flygare, Burlingame, CA (US); Rebecca Elizabeth Johnson, Oakland, CA (US); Simon B. Lang, San Francisco, CA (US); Christian L. Morales, Belmont, CA (US); Ryan Quiroz, San Francisco, CA (US); W. Michael Seganish, Castro Valley, CA (US); Ling Tong, Warren, NJ (US); Xin Wen, Burlingame, CA (US)

(73) Assignee: Merck Sharp & Dohme LLC, Rahway, NJ (US)

(21) Appl. No.: 19/121,528

(22) PCT Filed: Oct. 24, 2023
(86) PCT No.: PCT/US2023/077570

§ 371 Date: Apr. 16, 2025

Related U.S. Application Data
(60) Provisional application No. 63/419,236, filed on Oct. 25, 2022.

(51) Int. Cl. C07H 15/252; A61K 31/706; A61K 39/395

(52) U.S. Cl. CPC C07H 15/252; A61K 31/706; A61K 39/3955

(57) ABSTRACT

The present disclosure is directed to novel PNU Anthracycline Derivatives of formula (I); and pharmaceutically acceptable salts thereof, wherein R¹ and R² are as defined in the above disclosure. The disclosure is also directed to pharmaceutical compositions comprising the PNU Anthracycline Derivatives, and the use of these compounds and compositions in the prevention or treatment of cellular proliferative disorders.

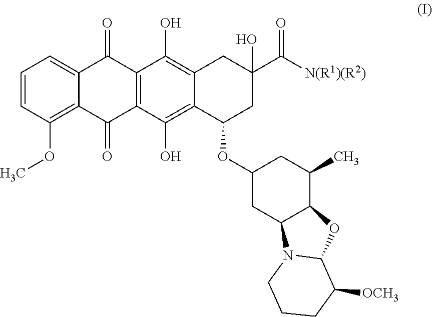

### FIELD OF THE INVENTION

The present disclosure relates to novel PNU Anthracycline Derivatives, compositions comprising at least one PNU Anthracycline Derivative, and methods of using the PNU Anthracycline Derivatives for treating or preventing a cellular proliferative disorder in a patient.

### BACKGROUND OF THE INVENTION

The global cancer burden has been continually rising and is predicted to keep growing throughout the coming decades, given population ageing and contemporary lifestyles. The American Cancer Society estimates that in 2022, 1.9 million new cancer cases will be diagnosed, and 609,360 cancer deaths will be recorded in the United States alone. The nearly 200 existing types of cancer share the fundamental hallmarks of uncontrolled growth and spread, due to the progressive acquirement of cellular capabilities of limitless proliferation and evasion from regulatory mechanisms.

Exploiting the rapid replication of cancer cells as a selective feature, numerous antineoplastic drugs, such as alkylating agents, antimetabolites, intercalators, polymerase inhibitors, and a vast majority of available therapeutic agents strategically disrupt nuclear mechanisms to kill tumors, though affecting healthy tissues with an inherent high cell renovation rate. Despite some weaknesses of cytotoxic agents, their discovery remains a milestone in anticancer therapy. Many old but effective drugs remain clinically relevant and, instead of being supplanted by modern targeted therapies, they are still used, especially in combination regimens.

Anthracyclines are a class of drugs used in cancer chemotherapy that are extracted from Streptomyces bacterium, and are among the most effective anticancer treatments ever developed, demonstrating efficacy against more types of cancer than any other class of chemotherapeutic agents. Clinically the most important anthracyclines are doxorubicin (adriamycin), daunorubicin (daunomycin), epirubicin and idarubicin. These compounds are used to treat many cancers, including leukemias, lymphomas, breast, stomach, uterine, ovarian, bladder cancer, and lung cancers.

Anthracycline drugs act mainly by intercalating with DNA and interfering with DNA metabolism and RNA production. Cytotoxicity is primarily due to inhibition of topoisomerase II after the enzyme induces a break in DNA, preventing religation of the break and leading to cell death. The basic structure of anthracyclines is that of a tetracyclic molecule with an anthraquinone backbone connected to a sugar moiety by a glycosidic linkage. When taken up by a cell, the four-ring structure intercalates between DNA bases pairs while the sugar sits within the minor groove and interacts with adjacent base pairs.

The usefulness of anthracyclines, however, is limited by their toxicity, with their main adverse effects being cardiotoxicity, severe neutropenia, and vomiting. Due to this toxicity, only a small number of anthracyclines or related DNA intercalating agents are available for clinical use.

Thus, there remains a need in the art for anthracycline compounds that can potentially treat cancer. The presently disclosed PNU Anthracycline Derivatives help address that need.

### SUMMARY OF THE INVENTION

In one aspect, provided are Compounds of Formula (I):

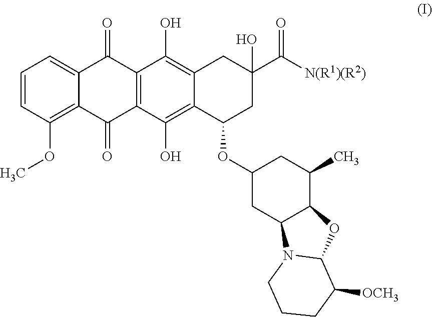

or a pharmaceutically acceptable salt thereof, wherein:
   R¹ is H or C₁₋₆ alkyl;
   R² is selected from C₁-C₆ hydroxyalkyl, 5 to 11-membered bridged bicyclic heterocycloalkyl, 5 to 11-membered fused bicyclic heterocycloalkyl, C₁-C₆ aminoalkyl, —(C₁-C₆ alkylene)-NR³C(O)—(C₁-C₆ aminoalkyl), —(C₁-C₆ alkylene)-NHC(O)NHNH₂, —(C₁-C₆ alkylene)-NR³, —(C₁-C₆ alkylene)-N(R³)—(C₁-C₆ alkylene)-N(R³)—NHC(O)—(C₁-C₆ aminoalkyl), —(C₁-C₆ haloalkylene)-NR³, —(C₁-C₆ alkylene)ₙ-(3 to 7-membered monocyclic heterocycloalkyl), —(C₁-C₆ alkylene)-NR³C(O)-(3 to 7-membered monocyclic heterocycloalkyl), —(C₁-C₆ alkylene)-NR³C(O)—(C₃-C₇ monocyclic cycloalkyl), —(C₁-C₆ alkylene)ₙ-(C₅-C₁₁ fused bicyclic cycloalkyl), —(C₁-C₆ alkylene)ₙ-(C₅-C₁₁ bridged bicyclic cycloalkyl), —(C₁-C₆ alkylene)ₙ-(C₃-C₇ monocyclic cycloalkyl), —(C₁-C₆ alkylene)ₙ-(C₆-C₁₀ aryl), —(C₁-C₆ alkylene)ₙ-(5 or 6-membered monocyclic heteroaryl), and 5-11 membered spirocyclic heterocycloalkyl, wherein said 3 to 7-membered monocyclic heterocycloalkyl group, said 5 to 10-membered fused bicyclic heterocycloalkyl group, said 5 to 10 membered bridged bicyclic heterocycloalkyl group, said C₃-C₇ monocyclic cycloalkyl group, said C₆-C₁₀ aryl group, said 5 or 6-membered monocyclic heteroaryl group, said C₅-C₁₁ bicyclic cycloalkyl group, and said 5-11 membered spirocyclic heterocycloalkyl group can each be optionally and independently substituted with one or more R^{A} groups, and wherein said 3 to 7-membered monocyclic heterocycloalkyl group can have one or more ring carbon atoms substituted with an oxo group:
alternatively, R¹ and R², taken together with the common nitrogen atom to which they are each attached, join to form: (i) a 3 to 7-membered monocyclic heterocycloalkyl group, (ii) a 5 to 11-membered bridged bicyclic heterocycloalkyl group, (iii) a 5 to 11-membered fused bicyclic heterocycloalkyl group, or (iv) a 5 to 11-membered spirocyclic heterocycloalkyl group, wherein said 3 to 7-membered monocyclic heterocycloalkyl group, said 5 to 11-membered bicyclic heterocycloalkyl group, said 5 to 11-membered fused heterocycloalkyl group, and said 5 to 11-membered spirocyclic heterocycloalkyl group can each be optionally and independently substituted with one or more R^{B} groups;
each occurrence of R³ is independently H or C₁₋₆ alkyl;
each occurrence of R^{A} is independently selected from C₁₋₆ alkyl, halo, —CN, —OR³, —(C₁-C₆ alkylene)ₙ-N(R³)₂, C₁-C₆ aminoalkyl, —(C₁-C₆ alkylene)-NHC(O)—(C₁-C₆ aminoalkyl), —O—(C₁₋₆ alkyl), —NHC(O)CH₂OH, and —(C₁-C₆ alkylene)-(3 to 7-membered monocyclic heterocycloalkyl), wherein a 3 to 7-membered monocyclic heterocycloalkyl group can be optionally and independently substituted with one or more R^{c} groups;
each occurrence of R^{B} is independently selected from C₁₋₆ alkyl, halo, —CN, —OR³, —N(R³)₂, C₁-C₆ aminoalkyl, —(C₁-C₆ alkylene)-NHC(O)—(C₁-C₆ aminoalkyl), —O—(C₁₋₆ alkyl), —NHC(O)CH₂OH, and —(C₁-C₆ alkylene)-(3 to 7-membered monocyclic heterocycloalkyl), wherein a 3 to 7-membered monocyclic heterocycloalkyl group can be optionally and independently substituted with one or more R^{C} groups;
each occurrence of R⁴ is independently selected from C₁₋₆ alkyl, halo, —CN, —OR³, —N(R³)₂, C₁-C₆ aminoalkyl, —O—(C₁₋₆ alkyl), —NHC(O)—(C₁-C₆ aminoalkyl), —(C₁-C₆ alkylene)-NHC(O)—(C₁-C₆ aminoalkyl), C₁₋₆ alkyl, halo, —CN, —OR³. —N(R³)₂, —O—(C₁₋₆ alkyl), and —NHC(O)CH₂OH; and each occurrence of n is independently 0 or 1.

The Compounds of Formula (I) (also referred to herein as the “Anthracycline Derivatives”), and pharmaceutically acceptable salts thereof, can be useful for treating or preventing a cellular proliferative disorder in a patient. Without being bound by any specific theory, it is believed that the Anthracycline Derivatives act as inhibitors of topoisomerases.

Accordingly, provided herein are methods for treating or preventing a cellular proliferative disorder in a patient, comprising administering to the patient an effective amount of at least one Anthracycline Derivative.

Further details are set forth in the accompanying detailed description below.

Although any methods and materials similar to those described herein can be used in the practice or testing of the Anthracycline Derivatives, illustrative methods and materials are now described. Other embodiments, aspects and features are either further described in or will be apparent from the ensuing description, examples and appended claims.

### DETAILED DESCRIPTION OF THE INVENTION

Described are novel Anthracycline Derivatives, compositions comprising at least one Anthracycline Derivative, and methods of using the Anthracycline Derivatives for treating or preventing a cellular proliferative disorder in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, it is to be understood that the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl.” “haloalkyl,” “—O-alkyl,” etc.

As used herein, and throughout this disclosure, the following terms, unless otherwise indicated, shall be understood to have the following meanings:

A “patient” is a human or non-human mammal. In one embodiment, a patient is a human.

The term “effective amount” as used herein, refers to an amount of PNU Anthracycline Derivative, and/or an additional therapeutic agent, or a composition thereof that is effective in producing the desired therapeutic, ameliorative, inhibitory or preventative effect when administered to a patient suffering from a cellular proliferative disorder. In the combination therapies described herein, an effective amount can refer to each individual agent or to the combination as a whole, wherein the amounts of all agents administered are together effective, but wherein the component agent of the combination may not be present individually in an effective amount.

The term “preventing,” as used herein with respect to a cellular proliferative disorder, refers to reducing the likelihood of a cellular proliferative disorder.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 10 carbon atoms. In different embodiments, an alkyl group contains from 1 to 10 carbon atoms (“C₁-C₁₀ alkyl”) or from about 1 to about 6 carbon atoms (“C₁-C₆ alkyl”). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, NH(cycloalkyl), —O—C(O)-alkyl, —O—C(O)-aryl, —O—C(O)— cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 10 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₁₀ alkenyl” refers to an alkenyl group having from 2 to 10 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 10 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)— alkyl, —O—C(O)-aryl, —O—C(O)-cycloalkyl, —C(O)OH and —C(O)O-alkyl. The term “C₂-C₁₀ alkynyl” refers to an alkynyl group having from 2 to 10 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 10 carbon atoms. In another embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “alkenylene,” as used herein, refers to an alkenyl group, as defined above, wherein one of the alkenyl group's hydrogen atoms has been replaced with a bond. Non-limiting examples of alkylene groups include —CH═CH—, —CH═CHCH₂—, —CH₂CH₂CH═CH—, and —CH₂(CH₃)C═CH—. In one embodiment, an alkenylene group has from 2 to about 6 carbon atoms. In one embodiment, an alkenylene group has from 2 to about 10 carbon atoms. In another embodiment, an alkenylene group is branched. In another embodiment, an alkenylene group is linear. The term “C₂-C₆ alkenylene” refers to an alkenylene group having from 2 to 6 carbon atoms.

The term “alkynylene,” as used herein, refers to an alkynyl group, as defined above, wherein one of the alkynyl group's hydrogen atoms has been replaced with a bond. Non-limiting examples of alkylene groups include —C≡C—, —C≡CCH₂—, and —C≡CCH(CH₃)₂—. In one embodiment, an alkynylene group has from 2 to about 6 carbon atoms. In another embodiment, an alkynylene group has from 2 to about 10 carbon atoms. In another embodiment, an alkynylene group is branched. In another embodiment, an alkynylene group is linear. The term “C₂-C₆ alkynylene” refers to an alkynylene group having from 2 to 6 carbon atoms. The term “C₂-C₁₀ alkynylene” refers to an alkynylene group having from 2 to 10 carbon atoms.

The term “aminoalkyl,” as used herein, refers to an alkyl group as defined above, wherein one of the alkyl group's hydrogen atoms has been replaced with —NH₂, —NH(C₁-C₆ alkyl), or —N(C₁-C₆ alkyl)₂. In one embodiment, an aminoalkyl group has from 1 to 6 carbon atoms. Non-limiting examples of aminoalkyl groups include —CH₂NH₂, —CH₂N(CH₃)₂, —CH₂CH₂NH₂, and —CH₂NH(CH)₃. The term “C₁-C₆ aminoalkyl” refers to an aminoalkyl group having from 1 to 6 carbon atoms.

The term “aryl,” as used herein, refers to an aromatic monocyclic or multicyclic ring system comprising from about 6 to about 14 carbon. In one embodiment, an aryl group contains from about 6 to about 10 carbon atoms (“C₆-C₁₀ aryl”). An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. An example of an aryl group fused to a cycloalkyl ring includes:

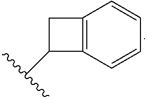 [SMILES: *C1Cc2ccccc21]

In one embodiment, an aryl group is phenyl. In another embodiment, an aryl group is napthalene. Unless otherwise indicated, an alkyl group is unsubstituted.

The term “cycloalkyl.” as used herein, refers to a non-aromatic mono- or multicyclic ring system comprising from about 3 to about 11 ring carbon atoms. In one embodiment, a cycloalkyl contains from about 5 to about 11 ring carbon atoms. In another embodiment, a cycloalkyl is monocyclic, and contains from about 3 to about 7 ring atoms. In another embodiment, a cycloalkyl is monocyclic, and contains from about 5 to about 6 ring atoms. In another embodiment, a cycloalkyl is bicyclic and contains about 4 to 10 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. Unless otherwise indicated, cycloalkyl group is unsubstituted. In one embodiment, a cycloalkyl group is unsubstituted. The term “3 to 7-membered monocyclic cycloalkyl” refers to a monocyclic cycloalkyl group having from 3 to 7 ring carbon atoms. The term “5 to 11-membered bicyclic cycloalkyl group” refers to a bicyclic cycloalkyl group having from 5 to 11 ring carbon atoms.

A multicyclic cycloalkyl group may have rings that are fused, rings that are joined in a spirocyclic manner, and rings that are bridged. In one embodiment, a cycloalkyl group can be a spirocyclic cycloalkyl group having from 5 to 11 ring carbon atoms (“C₅-C₁₁ spirocyclic cycloalkyl”). Illustrative examples of such a bicyclic cycloalkyl group include:

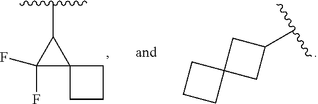 [SMILES: *C1C(F)(F)C12CCC2 ; *C1CC2(CCC2)C1]

In another embodiment, a cycloalkyl group can be a fused bicyclic cycloalkyl group having from 5 to 11 ring carbon atoms (“C₅-C₁₁ fused bicyclic cycloalkyl”). Illustrative examples of such a fused bicyclic cycloalkyl group include:

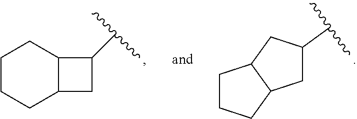 [SMILES: *C1CC2CCCCC12 ; *C1CC2CCCC2C1]

In another embodiment, a cycloalkyl group can be a bridged bicyclic cycloalkyl group having from 5 to 11 ring carbon atoms (“C₅-C₁₁ bridged bicyclic cycloalkyl”), or a bridged tricyclic cycloalkyl group having from 6 to 14 ring carbon atoms. Illustrative examples of such bridged bicyclic and tricyclic heterocycloalkyl groups include:

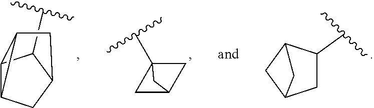 [SMILES: *C1C2CC3C(C2)C13 ; *C12CC(C1)C2 ; *C1CC2CC1C2]

A ring carbon atom of a cycloalkyl group may be functionalized as a carbonyl group. An illustrative example of such a cycloalkyl group (also referred to herein as a “cycloalkanoyl” group) includes, but is not limited to, cyclobutanol:

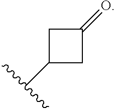 [SMILES: *C1CC(=O)C1]

The term “cycloalkenyl,” as used herein, refers to anon-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is cyclopentenyl. In another embodiment, a cycloalkenyl group is cyclohexenyl. The term “4 to 6-membered cycloalkenyl” refers to a cycloalkenyl group having from 4 to 6 ring carbon atoms.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 10 carbon atoms. In another embodiment, a haloalkyl group has from 1 to 6 carbon atoms. In another embodiment, a haloalkyl group is substituted with from 1 to 6 F atoms. In a class of this embodiment, the haloalkyl group is substituted with from 1 to 3 F atoms. Non-limiting examples of haloalkyl groups include —CH₂CHF₂, —CH₂F, —CHF₂, —CF₃, —CH₂Cl and —CCl₃. The term “C₁-C₆ haloalkyl” refers to a haloalkyl group having from 1 to 6 carbon atoms.

The term “haloalkylene,” as used herein, refers to a haloalkyl group as defined above, wherein one or more of the haloalkyl group's hydrogen atoms has been replaced with a halogen. In one embodiment, a haloalkylene group has from 1 to 10 carbon atoms. In another embodiment, a haloalkylene group has from 1 to 6 carbon atoms. In another embodiment, a haloalkylene group is substituted with from 1 to 6 F atoms. In a class of this embodiment, the haloalkylene group is substituted with from 1 to 3 F atoms. Non-limiting examples of haloalkylene groups include —CH₂CHF₂, —CH₂F. —CHF₂, —CF₃, —CH₂Cl and —CCl₃. The term “C₁-C₆ haloalkylene” refers to a haloalkylene group having from 1 to 6 carbon atoms.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 10 carbon atoms. In another embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₁₀ hydroxyalkyl” refers to a hydroxyalkyl group having from 1 to 10 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms (“5 or 6-membered monocyclic heteroaryl”). In another embodiment, a heteroaryl group is bicyclic and had 8 to 10 ring atoms (“8 to 10-membered bicyclic heteroaryl”). In still another embodiment, a heteroaryl group is bicyclic and has 9 or 10 ring atoms (“9 or 10-membered bicyclic heteroaryl”). A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl, such as pyridyl.

In one embodiment, an 8 to 10-membered bicyclic heteroaryl group comprises a fused bicyclic heterocyclic group in which one of the two fused rings is phenyl or monocyclic heteroaryl, such as:

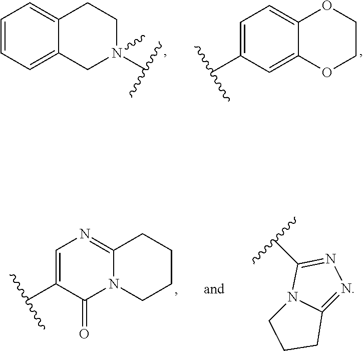

A “9 to 14-membered tricyclic heteroaryl” comprises an 8 to 10-membered bicyclic heteroaryl group, wherein a third ring is fused to one of the rings of the 8 to 10-membered bicyclic heteroaryl group. Such third ring can be a cycloalkyl, heterocycloalkyl, or heteroaryl ring. Examples of a 9 to 14-membered tricyclic heteroaryl group include:

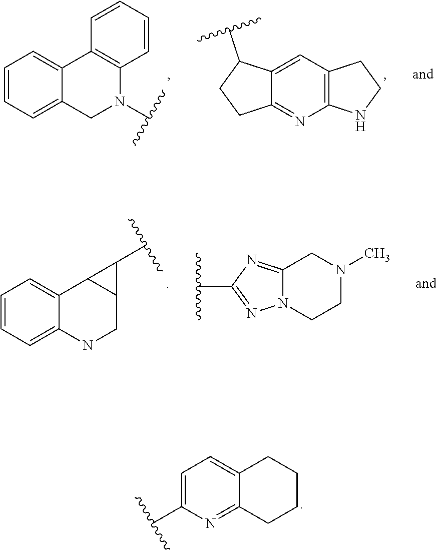 [SMILES: *N1Cc2ccccc2-c2ccccc21 ; *C1CCc2nc3c(cc21)CCN3 ; *C1C2CNc3ccccc3C12 ; *c1nc2n(n1)CCN(C)C2 ; *c1ccc2c(n1)CCCC2]

The term “heterocycloalkyl,” as used herein, refers to a non-aromatic saturated monocyclic or multicyclic ring system comprising 3 to about 14 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic. In one embodiment, a heterocycloalkyl group is monocyclic and has from about 3 to about 7 ring atoms (“3 to 7-membered monocyclic heterocycloalkyl”). In another embodiment, a heterocycloalkyl group is monocyclic and has 5 or 6 ring atoms (“5 or 6-membered monocyclic heterocycloalkyl”). In one embodiment, a heterocycloalkyl group is bicyclic. In another embodiment, a heterocycloalkyl group is bicyclic and has from about 5 to about 11 ring atoms (“5 to 11-membered bicyclic heterocycloalkyl”). In another embodiment, a heterocycloalkyl group is tricyclic and has from about 10 to about 14 ring atoms (“10 to 14-membered tricyclic heterocycloalkyl”). There are no adjacent oxygen and/or sulfur atoms present in the ring system. Any —NH group in a heterocycloalkyl ring may exist protected such as, for example, as an —N(BOC), —N(CBz). —N(Tos) group and the like; such protected heterocycloalkyl groups are considered part of the present disclosure. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

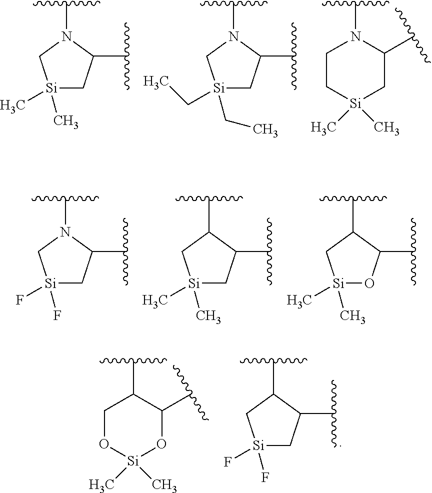

A ring carbon atom of a heterocycloalkyl group may be functionalized as a carbonyl group (“oxo”). Illustrative examples of such a heterocycloalkyl group include, but are not limited to:

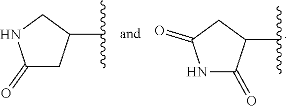 [SMILES: *C1CNC(=O)C1 ; *C1CC(=O)NC1=O]

A ring sulfur atom of a heterocycloalkyl group may also be functionalized as a sulfonyl group. An example of such a heterocycloalkyl group is:

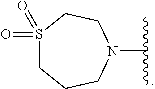 [SMILES: *N1CCCS(=O)(=O)CC1]

In one embodiment, a heterocycloalkyl group is a 5-membered monocyclic heterocycloalkyl. In another embodiment, a heterocycloalkyl group is a 6-membered monocyclic heterocycloalkyl.

A multicyclic heterocycloalkyl group may have rings that are fused, rings that are joined in a spirocyclic manner, and rings that are bridged. In one embodiment, a heterocycloalkyl group can be a bicyclic spirocyclic heteroaryl group having from 1 to 11 ring atoms. Illustrative examples of such a bicyclic heterocycloalkyl group include:

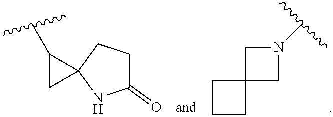 [SMILES: *C1CC12CCC(=O)N2 ; "and" ; *N1CC2(CCC2)C1]

In another embodiment, a heterocycloalkyl group can be a fused bicyclic heterocycloalkyl group having from 5 to 11 ring atoms (“5 to 1 l-membered fused bicyclic heterocycloalkyl”). Illustrative examples of such a fused bicyclic heterocycloalkyl group include:

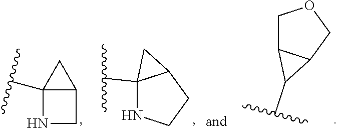

In another embodiment, a heterocycloalkyl group can be a bridged heterocycloalkyl group having from 5 to 11 ring atoms (“5 to 11-membered bridged bicyclic heterocycloalkyl”).

Illustrative examples of such a bridged bicyclic heterocycloalkyl group include:

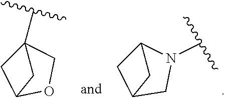 [SMILES: *C12COC(C1)C2 ; *N1CC2CC1C2]

The term “heterocycloalkenyl.” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 6 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide. S-oxide or S,S-dioxide. A ring carbon atom of a heterocycloalkenyl group may be functionalized as a carbonyl group. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl. 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. In one embodiment, a heterocycloalkenyl group is a 5-membered heterocycloalkenyl. In another embodiment, a heterocycloalkenyl group is a 6-membered heterocycloalkenyl. The term “4 to 6-membered heterocycloalkenyl” refers to a heterocycloalkenyl group having from 4 to 6 ring atoms.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, Greene et al., Protective Groups in Organic Synthesis, Wiley-Interscience, New York, (1999).

Examples of “ring system substituents” include, but are not limited to, alkyl, alkenyl, alknyl, aryl, heteroaryl, -alkylene-aryl, -arylene-alkyl, -alkylene-heteroaryl,-alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkyleneheteroaryl, —S(O)₂-alkylene-aryl, —S(O)₂-alkylene-heteroaryl, —Si(alkyl)₂, —Si(aryl)₂, Si(heteroaryl)₂-Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y¹)(Y²), -alkylene-N(Y¹)(Y²), —C(O)N(Y′)(Y²), and —S(O)₂N(Y¹)(Y²), wherein Y¹ and Y² can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

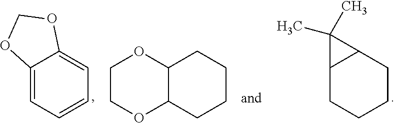

When any substituent or variable (e.g., R⁵, n, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

Prodrugs and solvates of the compounds of the present disclosure are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a PNU Anthracycline Derivative or a pharmaceutically acceptable salt or solvate of the compound. The transformation may occur by various mechanisms (e.g., by metabolic or chemical processes), such as, for example, through hydrolysis in blood.

For example, if a PNU Anthracycline Derivative or a pharmaceutically acceptable salt, hydrate or solvate thereof contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 6 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a PNU Anthracycline Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a PNU Anthracycline Derivative incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino): (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the present disclosure may exist in unsolvated as well as solvated forms with pharmaceutically acceptable solvents such as water, ethanol, and the like, and it is intended that the present disclosure embrace both solvated and unsolvated forms. “Solvate” means a physical association of a compound of the present disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the present disclosure may optionally be converted to a solvate. Preparation of solvates is generally known. Thus, for example, M. Caira et al, J Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The PNU Anthracycline Derivatives can form salts which are also within the scope of the present disclosure. The term “salt(s)”, as employed herein, denotes acidic salts formed with inorganic and/or organic acids, as well as basic salts formed with inorganic and/or organic bases. In addition, when a PNU Anthracycline Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a PNU Anthracycline Derivative with an amount of acid or base, such as an equivalent amount, in a medium such as one in which the salt precipitates or in an aqueous medium followed by lyophilization.

Exemplary acid addition salts include acetates, ammonium, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (also known as mesylates), naphthalenesulfonates, nitrates, oxalates, phosphates, propionates, salicylates, succinates, sulfates, tartarates, thiocyanates, toluenesulfonates (also known as tosylates), and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto. In one embodiment, an acid salt is an ammonium salt or a di-ammonium salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the present disclosure and all acid and base salts are considered equivalent to the free forms of the corresponding compounds for purposes of the present disclosure.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the PNU Anthracycline Derivatives may be atropisomers (e.g., substituted biaryls), and are considered as part of the present disclosure. Enantiomers can also be directly separated using chiral chromatographic techniques.

It is also possible that the PNU Anthracycline Derivatives may exist in different tautomeric forms, and all such forms are embraced within the scope of the present disclosure. For example, all keto-enol and imine-enamine forms of the compounds are included in the present disclosure.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of the present disclosure. If a PNU Anthracycline Derivative incorporates a double bond or a fused ring, both the cis- and trans-forms, as well as mixtures, are embraced within the scope of the present disclosure.

Individual stereoisomers of the compounds of the present disclosure may, for example, be substantially free of other isomers, or may be admixed, for example, as racemates or with all other, or other selected, stereoisomers. The chiral centers of the present disclosure can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of generic Formula I. For example, different isotopic forms of hydrogen (H) include protium (¹H), and deuterium (²H). Protium is the predominant hydrogen isotope found in nature. Enriching for deuterium may provide certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the PNU Anthracycline Derivatives, and of the salts, solvates, hydrates, esters and prodrugs of the PNU Anthracycline Derivatives, are intended to be included in the present disclosure.

The following abbreviations are used below and have the following meanings:

| | |
|---|---|
| aq | Aqueous |
| Boc₂O | di-tert-butyl dicarbonate |
| CDI | Carbonyldiimidazole |
| Celite | Diatomaceous earth |
| CH₃I | iodomethane |
| DAST | Diethylaminosulfur trifluoride |
| DCE | Dichloroethane |
| DCM | Dichloromethane |
| DIEA or DIPEA | N,N-Diisopropylethylamine |
| DMAP | 4-Dimethylaminopyridine |
| DMF | Dimethylformamide |
| DMSO | Dimethyl sulfoxide |
| EDC | 1-Ethyl-3-[3-dimethylaminopropyl]carbodiimide |
| | hydrochloride |
| ESI | Electron-spray ionization |
| EtOAc | Ethyl acetate |
| Et₃N | Triethylamine |
| h | Hours |
| HATU | (1-[Bis(dimethylamino)methylene]-1H-1,2,3-triazolo[4,5-b]pyridinium |
| | 3-oxide hexafluorophosphate |
| ¹H-NMR | Proton nuclear magnetic resonance |
| HCl | Hydrochloric acid |
| HPLC | High performance liquid chromatography |
| IPA | Isopropyl alcohol |
| KHMDS | Potassium bis(trimethylsilyl)amide |
| LAH | Lithium aluminum hydride |
| LCMS | Liquid chromatography-mass spectrometry |
| LiOH | Lithium hydroxide |
| MgSO₄ | Magnesium Sulfate |
| MeCN | Acetonitrile |
| MeOH | Methanol |
| MS | Mass spectrometry |
| m/z | Mass to charge ratio |
| Na₂SO₄ | Sodium sulfate |
| NaHCO₃ | Sodium bicarbonate |
| NaBH₄ | Sodium borohydride |
| NaH | Sodium hydride |
| Pd | Palladium |
| Pd/C | Palladium on Carbon |
| SFC | Supercritical Fluid Chromatography |
| TBAI | Tetra-n-butylammonium iodide |
| TBSCl | tert-Butyldimethylsilyl chloride |
| TEA | triethylamine |
| TFA | Trifluoroacetic acid |
| THF | Tetrahydrofuran |
| TLC | Thin Layer Chromatography |
| t_{R} | Retention time |
| trt | Triphenyl methyl |

### The Compounds of Formula (I)

Provided herein are PNU Anthracycline Derivatives of formula (I):

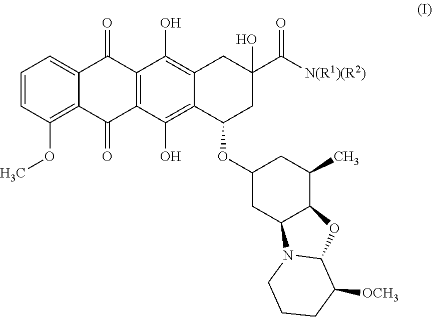

and pharmaceutically acceptable salts and solvates thereof, wherein:
   R¹ is H or C₁₋₆ alkyl;
   R² is selected from C₁-C₆ hydroxyalkyl, 5 to 11-membered bridged bicyclic heterocycloalkyl, 5 to 11-membered fused bicyclic heterocycloalkyl, C₁-C₆ aminoalkyl, —(C₁-C₆ alkylene)-NR³C(O)—(C₁-C₆ aminoalkyl), —(C₁-C₆ alkylene)-NHC(O)NHNH₂, —(C₁-C₆ alkylene)-NR³, —(C₁-C₆ alkylene)-N(R)—(C₁-C₆ alkylene)-N(R³)—NHC(O)—(C₁-C₆ aminoalkyl), —(C₁-C₆ haloalkylene)-NR³, —(C₁-C₆ alkylene)ₙ-(3 to 7-membered monocyclic heterocycloalkyl), —(C₁-C₆ alkylene)-NR³C(O)-(3 to 7-membered monocyclic heterocycloalkyl), —(C₁-C₆ alkylene)-NR³C(O)—(C₃-C₇ monocyclic cycloalkyl), —(C₁-C₆ alkylene)ₙ-(C₅-C₁ fused bicyclic cycloalkyl), —(C₁-C₆ alkylene)ₙ-(C₅-C₁₁ bridged bicyclic cycloalkyl), —(C₁-C₆ alkylene)ₙ-(C₃-C₇ monocyclic cycloalkyl), —(C₁-C₆ alkylene)ₙ-(C₆-C₁₀ aryl), —(C₁-C₆ alkylene)ₙ-(5 or 6-membered monocyclic heteroaryl), and 5-11 membered spirocyclic heterocycloalkyl, wherein said 3 to 7-membered monocyclic heterocycloalkyl group, said 5 to 10-membered fused bicyclic heterocycloalkyl group, said 5 to 10 membered bridged bicyclic heterocycloalkyl group, said C₃-C₇ monocyclic cycloalkyl group, said C₆-C₁₀ aryl group, said 5 or 6-membered monocyclic heteroaryl group, said C₅-C₁₁ bicyclic cycloalkyl group, and said 5-11 membered spirocyclic heterocycloalkyl group can each be optionally and independently substituted with one or more R^{A} groups, and wherein said 3 to 7-membered monocyclic heterocycloalkyl group can have one or more ring carbon atoms substituted with an oxo group;
alternatively, R¹ and R², taken together with the common nitrogen atom to which they are each attached, join to form: (i) a 3 to 7-membered monocyclic heterocycloalkyl group, (ii) a 5 to 11-membered bridged bicyclic heterocycloalkyl group, (iii) a 5 to 11-membered fused bicyclic heterocycloalkyl group, or (iv) a 5 to 11-membered spirocyclic heterocycloalkyl group, wherein said 3 to 7-membered monocyclic heterocycloalkyl group, said 5 to 11-membered bicyclic heterocycloalkyl group, said 5 to 11-membered fused heterocycloalkyl group, and said 5 to 11-membered spirocyclic heterocycloalkyl group can each be optionally and independently substituted with one or more R^{B} groups;
each occurrence of R³ is independently H or C₁₋₆ alkyl;
each occurrence of R^{A} is independently selected from C₁₋₆ alkyl, halo, —CN, —OR³, —(C₁-C₆ alkylene)ₙ-N(R³)₂, C₁-C₆ aminoalkyl, —(C₁-C₆ alkylene)-NHC(O)—(C₁-C₆ aminoalkyl), —O—(C₁₋₆ alkyl), —NHC(O)CH₂OH, and —(C₁-C₆ alkylene)-(3 to 7-membered monocyclic heterocycloalkyl), wherein a 3 to 7-membered monocyclic heterocycloalkyl group can be optionally and independently substituted with one or more R^{c} groups;
each occurrence of R^{B} is independently selected from C₁₋₆ alkyl, halo, —CN, —OR³, —N(R³)₂, C₁-C₆ aminoalkyl, —(C₁-C₆ alkylene)-NHC(O)—(C₁-C₆ aminoalkyl), —O—(C₁₋₆ alkyl), —NHC(O)CH₂OH, and —(C₁-C₆ alkylene)-(3 to 7-membered monocyclic heterocycloalkyl), wherein a 3 to 7-membered monocyclic heterocycloalkyl group can be optionally and independently substituted with one or more R^{c} groups;
each occurrence of R^{c} is independently selected from C₁₋₆ alkyl, halo, —CN, —OR³, —N(R³)₂, C₁-C₆ aminoalkyl, —O—(C₁₋₆ alkyl), —NHC(O)—(C₁-C₆ aminoalkyl), —(C₁-C₆ alkylene)-NHC(O)—(C₁-C₆ aminoalkyl), C₁₋₆ alkyl, halo, —CN, —OR³, —N(R³)₂, —O—(C₁₋₆ alkyl), and —NHC(O)CH₂OH; and
each occurrence of n is independently 0 or 1.

In one embodiment, the compounds of formula (I) have the formula (Ia):

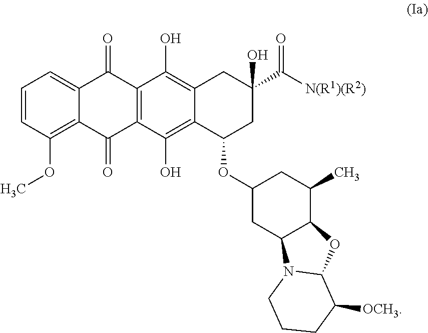

In another embodiment, the compounds of formula (I) have the formula (Ib):

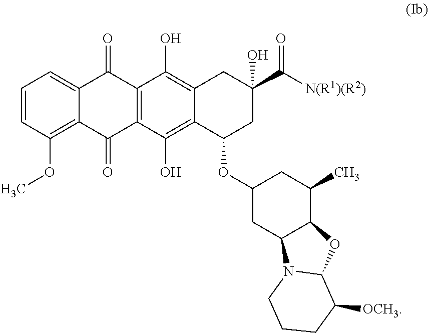

In one embodiment. R¹ is H, and R² is selected from C₃-C₇ monocyclic cycloalkyl, C₅-C₁₁ fused bicyclic cycloalkyl, C₅-C₁₁ bridged bicyclic cycloalkyl. C₆-C₁₀ aryl, 3 to 7-membered monocyclic heterocycloalkyl, 5 to 11-membered bridged bicyclic heterocycloalkyl, 5 to 11-membered fused bicyclic heterocycloalkyl, 5-11 membered spirocyclic heterocycloalkyl, and 5 or 6-membered monocyclic heteroaryl, wherein said C₃-C₇ monocyclic cycloalkyl group, said C₅-C₁₁ fused bicyclic cycloalkyl group, said C₅-C₁₁ bridged bicyclic cycloalkyl group, said C₆-C₁₀ aryl group, said 3 to 7-membered monocyclic heterocycloalkyl group, said 5 to 10-membered fused bicyclic heterocycloalkyl group, said 5 to 10 membered bridged bicyclic heterocycloalkyl group, said 5-11 membered spirocyclic heterocycloalkyl group, and said 5 or 6-membered heteroaryl group can each be optionally and independently substituted with one or more R^{A} groups.

In another embodiment. R¹ is H, and R² is selected from:

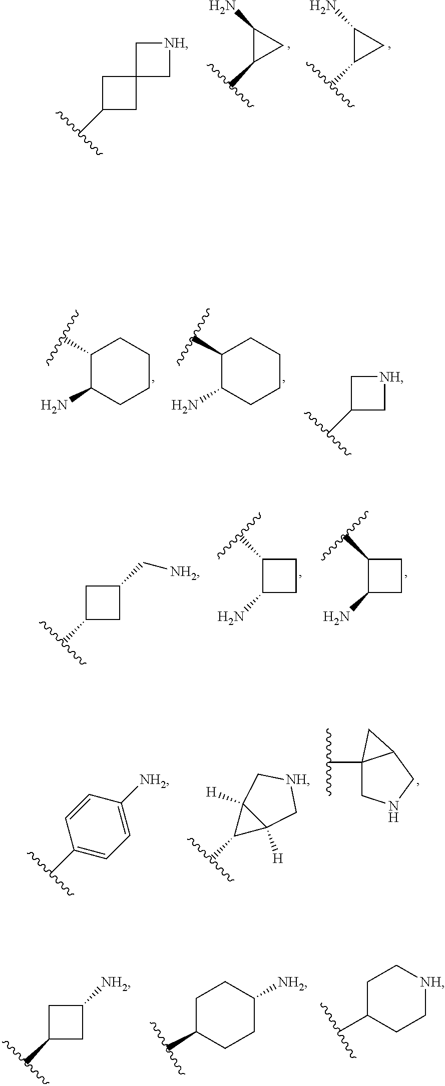

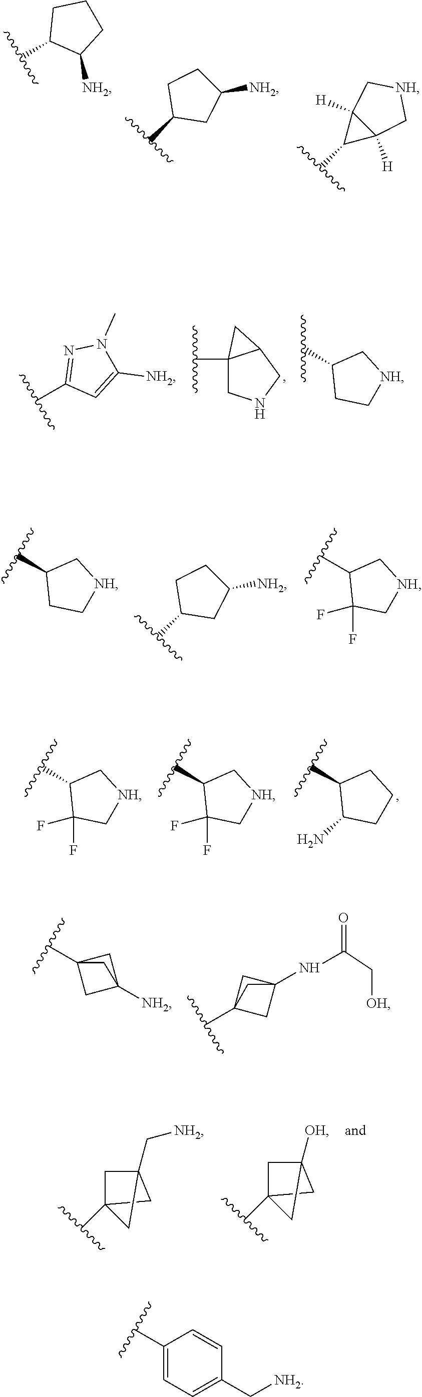

In another embodiment, R¹ is H, and R² is selected from C₁-C₆ hydroxyalkyl, C—C₆ aminoalkyl), —(C₁-C₆ alkylene)-NR³C(O)—(C₃-C₆ aminoalkyl), —(C₁-C₆ alkylene)-NHC(O)NHNH₂—(C₁-C₆ alkylene)-N(R³)—(C₁-C₆ alkylene)-N(R³)—NRHC(O)—(C₁-C₆ aminoalkyl), and —(C₁-C₆ haloalkylene)-NR³.

In a further embodiment, R¹ is H. and R² is selected from —CH₂CH₂NHC(O)CH₂NH₂, —CH₂CH₂NH—C(O)NHNH₂, —CH₂CH₂NHC(O)CH(CH₃)NH₂, —(CH₂)₃NH(CH₂)₃NHC(O)CH₂NH₂, —CH₂CH₂N(CH₃)C(O)C(CH₃)(NH₂)CH(CH₃)₂, —CH₂CH₂NHCH₃, —(CH₂)₄NH₁₂, —CH₂C(CH₃)₂CH₂NH₂, —(CH₂)₃N(CH₃)CH₂CH₂CH₂NH₂, —CH₂C(CH₃)₂NH₂, —(CH₂)₃NH-2, —CH₂CH(NH₂)CF₃, —CH₂CH(NH₂)CHF₂, —CH₂CH₂CH(NH₂)CF₃, —CH₂CH₂CH(NH₂)CHF₂, —(CH₂)₃NHCH₃, —CH₂CH₂OH, and —CH₂NH₂.

In one embodiment, R¹ is H, and R² is selected from —(C₁-C₆ alkylene)-(3 to 7-membered monocyclic heterocycloalkyl), —(C₁-C₆ alkylene)-NR³C(O)-(3 to 7-membered monocyclic heterocycloalkyl), —(C₁-C₆ alkylene)-NR³C(O)—(C₃-C₇ monocyclic cycloalkyl). —(C₁—C₆ alkylene)-(C₅-C₁₁ bridged bicyclic cycloalkyl group), —(C₁-C₆ alkylene)-(C₃-C₇ monocyclic cycloalkyl), —(C₁-C₆ alkylene)-(C₆-C₁₀ aryl), and —(C₁-C₆ alkylene)-(5 or 6-membered monocyclic heteroaryl), wherein any of said 3 to 7-membered monocyclic heterocycloalkyl groups, any of said C₃-C₇ monocyclic cycloalkyl groups, said C₆-C₁₀ aryl group, and said 5 or 6-membered monocyclic heteroaryl group can each be optionally and independently substituted with one or more R^{A} groups.

In another embodiment, R¹ is H, and R² is selected from:

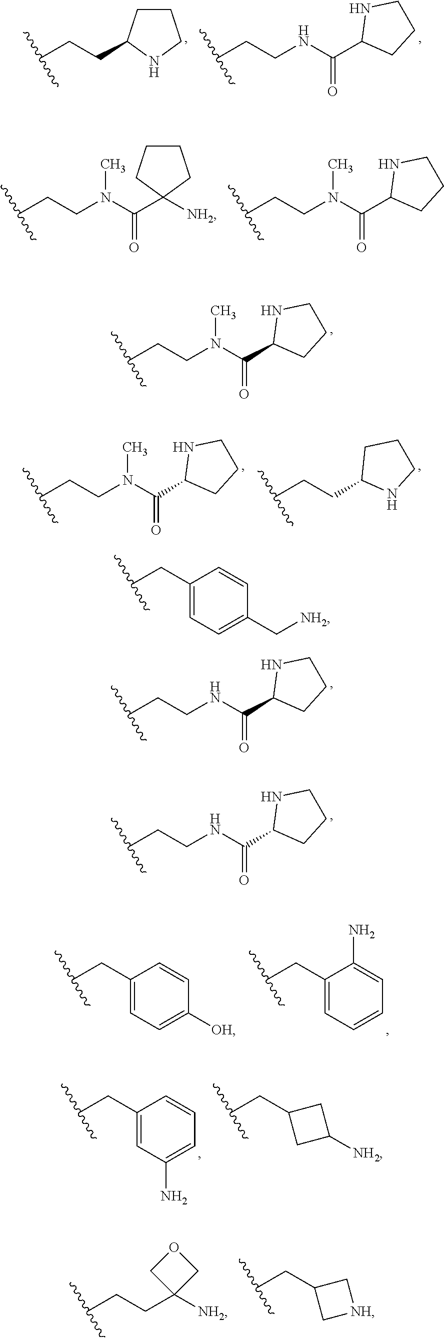

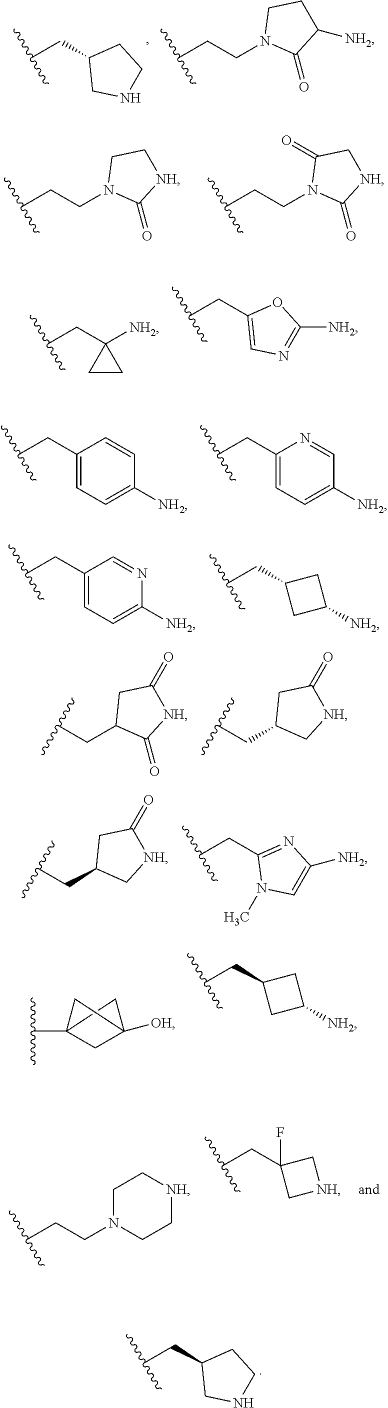

In one embodiment, R¹ and R², taken together with the common nitrogen atom to which they are each attached, join to form a 3 to 7-membered monocyclic heterocycloalkyl group, 5 to 11-membered bridged bicyclic heterocycloalkyl group, or a 5 to 11-membered fused bicyclic heterocycloalkyl group, wherein said 3 to 7-membered monocyclic heterocycloalkyl group, said 5 to 11-membered bridged bicyclic heterocycloalkyl group, and said 5 to 11-membered fused bicyclic heterocycloalkyl group can each be optionally and independently substituted with one or more R^{B} groups.

In another embodiment. R¹ and R², taken together with the common nitrogen atom to which they are each attached, join to form group selected from the following:

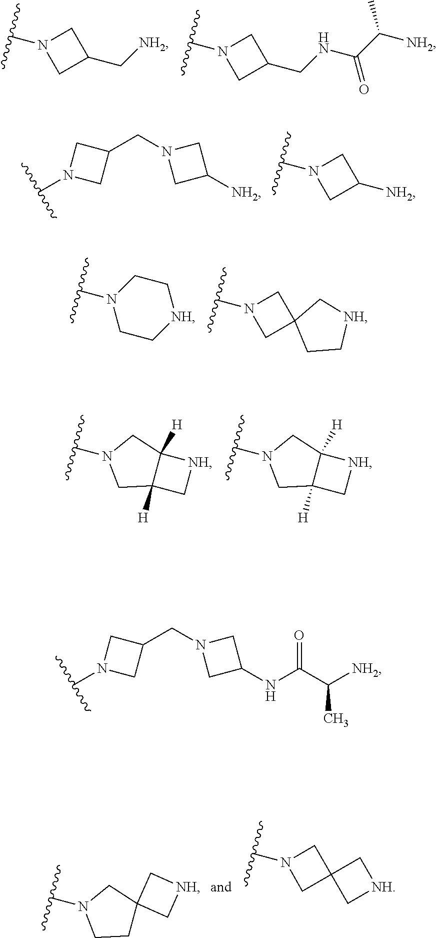

In one embodiment, for the compounds of formula (I), (Ia), and (Ib). R¹ is H, and R² is selected from —(C₁-C₁₀ alkylene)-(C₆-C₁₀ aryl), —(C₁-C₁₀ alkylene)-(3 to 7-membered monocyclic heterocycloalkyl), 5 to 10-membered bicyclic cycloalkyl, —(C₁-C₁₀ alkylene)-(C₃-C₇ monocyclic cycloalkyl); and 5 or 6-membered monocyclic heteroaryl, wherein said C₆-C₁₀ aryl group, said 3 to 7-membered monocyclic heterocycloalkyl group, said 5 to 10-membered bicyclic cycloalkyl group, said C₃-C₇ monocyclic cycloalkyl group; and said 5 or 6-membered monocyclic heteroaryl group can be optionally substituted by —NH₂.

In another embodiment, for the compounds of formula (I), (Ia), and (Ib), R¹ is H, and R² is selected from:

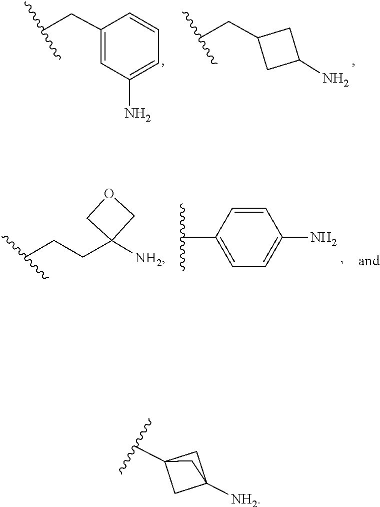

In one embodiment, the compound of formula (I) is any of the compounds numbered 1-90 in the instant specification, or a pharmaceutically acceptable salt thereof.

In one embodiment, the compound of formula (I) is in substantially purified form.

Other embodiments include the following:
(a) A pharmaceutical composition comprising an effective amount of a PNU Anthracycline Derivative, and a pharmaceutically acceptable carrier.
(b) The pharmaceutical composition of (a), further comprising a second therapeutic agent selected from the group consisting of anticancer agents.
(c) The pharmaceutical composition of (b), wherein the anticancer agent is an anti-human PD-1 antibody (or antigen-binding fragment thereof).
(d) A pharmaceutical combination that comprises: (i) a PNU Anthracycline Derivative, and (ii) a second therapeutic agent selected from the group consisting of anticancer agents, wherein the PNU Anthracycline Derivative, and the second therapeutic agent are each employed in an amount that renders the combination effective for inhibiting replication of cancer cells, or for treating cancer and/or reducing the likelihood or severity of symptoms of cancer.
(e) The combination of (d), wherein the second therapeutic agent is an anti-human PD-1 antibody (or antigen-binding fragment thereof).
(f) A method of inhibiting cancer cell replication in a subject in need thereof which comprises administering to the subject an effective amount of a PNU Anthracycline Derivative.
(g) A method of treating cancer and/or reducing the likelihood or severity of symptoms of cancer in a subject in need thereof which comprises administering to the subject an effective amount of a PNU Anthracycline Derivative.
(h) The method of (g), wherein the PNU Anthracycline Derivative is administered in combination with an effective amount of at least one second therapeutic agent selected from the group consisting of anticancer agents.
(i) The method of (h), wherein the second therapeutic agent is an anti-human PD-1 antibody (or antigen-binding fragment thereof).
(j) A method of inhibiting cancer cell replication in a subject in need thereof which comprises administering to the subject the pharmaceutical composition of (a), (b) or (c) or the combination of (d) or (e).
(k) A method of treating cancer and/or reducing the likelihood or severity of symptoms of cancer in a subject in need thereof which comprises administering to the subject the pharmaceutical composition of (a), (b) or (c) or the combination of (d) or (e).

Also described herein are PNU Anthracycline Derivative for use (i) in, (ii) as a medicament for, or (iii) in the preparation of a medicament for: (a) medicine; (b) inhibiting cancer cell replication, or (c) treating cancer and/or reducing the likelihood or severity of symptoms of cancer. In these uses, the PNU Anthracycline Derivative can optionally be employed in combination with one or more additional therapeutic agents selected from anticancer agents.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 1-90, as set forth in the Examples below, and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below Alternative synthetic pathways and analogous structures will be apparent to those skilled in the art of organic synthesis.

One skilled in the art of organic synthesis will recognize that the synthesis of the bicyclic heterocycle cores contained in Compounds of Formula (I) may require protection of certain functional groups (i.e., derivatization for the purpose of chemical compatibility with a particular reaction condition). Suitable protecting groups for the various functional groups of these Compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the bicyclic heterocycle cores of the Compounds of Formula (I) may be more desirable depending on the choice of appendage substituents.

Additionally, one skilled in the art will recognize that in some cases the order of reactions may differ from that presented herein to avoid functional group incompatibilities and thus adjust the synthetic route accordingly.

The preparation of multicyclic intermediates useful for making the bicyclic heterocycle cores of the Compounds of Formula (I) have been described in the literature and in compendia such as “Comprehensive Heterocyclic Chemistry” editions I, II and III, published by Elsevier and edited by A. R. Katritzky & R. J K Taylor. Manipulation of the required substitution patterns have also been described in the available chemical literature as summarized in compendia such as “Comprehensive Organic Chemistry” published by Elsevier and edited by D H R. Barton and W. D. Ollis; “Comprehensive Organic Functional Group Transformations” edited by edited by A. R. Katritzky & R. J K Taylor and “Comprehensive Organic Transformation” 3^{rd} Edition, published by Wiley-CVH and edited by R. C. Larock.

The starting materials used, and the intermediates prepared using the methods set forth in the Examples below may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analytical Development for Low-dose Oral Drug Products,” Wiley, 2009, ISBN.

### EXAMPLES

### General Methods

Solvents, reagents, and intermediates that are commercially available were used as received. Reagents and intermediates that are not commercially available were prepared in the manner as described below. ¹H NMR spectra are reported as ppm from residual solvent with number of protons, multiplicities, and coupling constants in Hertz indicated parenthetically.

Where LC/MS data are presented, the observed parent ions are given. Unless otherwise noted, flash chromatography is carried out on an Isco, Analogix, or Biotage automated chromatography system using a commercially available silica gel cartridge as the column. Reverse phase prep-HPLC conditions are described herein. When an aqueous solution was concentrated, concentration was achieved using a Genevac evaporator or lyophilization.

Reaction progress and synthetic intermediate analysis were assessed by LCMS (UV detection with ESI, APCI, or other mass detection) when applicable using a MeCN/water gradient with either TFA, formic acid, or NH₄HCO₃ modifier. Silica gel and reverse-phase flash column chromatography were conducted with commercially available pre-packed columns. Reverse-phase preparative HPLC purification was performed on preparative HPLC instruments with UV and MS detection using a MeCN/water gradient with either TFA, formic acid, or NH₄OH modifier. ¹H NMR spectra were collected at room temperature, and chemical shifts are reported in ppm relative to the residual proteo-solvent signals, and multiplicities, coupling constants (where applicable), and signal integrations are listed parenthetically. Unless otherwise noted, all EC₅₀ data presented in tables refers to the CellTiter-Glo® 2.0 Cytotoxicity Assay that is described in the Biological Assay section.

### Preparation of Intermediate Compounds

The following Examples 1-10 describe the synthesis of intermediate compounds useful for making the illustrative compounds of the present disclosure.

### Example 1

### Preparation of Intermediate Compound vi

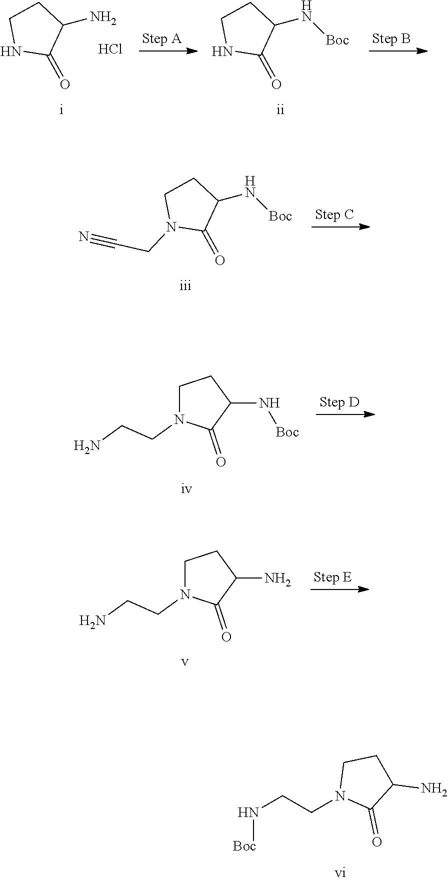

### Step A—Synthesis of Compound ii

To a solution of 3-aminopyrrolidin-2-one (i, 50.0 mg, 0.499 mmol) and Boc₂O (0.174 mL, 0.749 mmol) in MeOH (2 mL) was added TEA (0.209 mL, 1.50 mmol), and the resulting reaction was allowed to stir at 70° C. for 16 hours. The reaction mixture was then concentrated in vacuo, and the resulting residue was purified using flash silica gel chromatography (0-100% EtOAc/Petroleum ether) to provide tert-butyl (2-oxopyrrolidin-3-yl)carbamate ii. MS (ESI) m/z: 201.3 [M+H]⁺

### Step B—Synthesis of Compound iii

To a stirred suspension of NaH (24 mg, 0.60 mmol) in THF (1 mL) was added compound ii (100 mg, 0.499 mmol) at 0° C., and the resulting reaction was allowed to stir at room temperature for 30 minutes under N₂ atmosphere. A solution of 2-bromoacetonitrile (71.9 mg, 0.599 mmol) and TBAI (36.9 mg, 0.100 mmol) in THF (1 mL) was then added, and the resulting reaction was allowed to stir at 25° C. for 3 hours. Water (30 mL) was added, and the resulting mixture was extracted with ethyl acetate (100 mL×2). The combined organic extracts were washed with brine (100 mL×2), dried over Na₂SO₄, filtered and concentrated in vacuo. The residue obtained was purified using flash silica gel chromatography (0-100% EtOAc/Petroleum) to provide tert-butyl (1-(cyanomethyl)-2-oxopyrrolidin-3-yl)carbamate iii.

### Step C—Synthesis of Compound iv

To a solution of iii (1.3 g. 5.4 mmol) in EtOH (15 mL) was added platinum(IV) oxide (0.247 g, 1.09 mmol) and the resulting reaction was allowed to stir at 25° C. for 16 hours under H₂ atmosphere (obtained using a H₂ filled balloon). The reaction mixture was filtered, and the filter cake was washed with ethanol (10 mL). The combined filtrate and washing was concentrated in vacuo, and the residue obtained was purified using preparative HPLC (Column Welch Xtimate C18 150×25 mm×5 μm, 8 to 100% ACN:water (with TFA as modifier)), then lyophilized to provide tert-butyl (1-(2-aminoethyl)-2-oxopyrrolidin-3-yl)carbamate iv. MS (ESI) m/z: 244.2 [M+H⁺].

### Step D—Synthesis of Compound v

To a solution of iv (400 mg, 1.64 mmol) in DCM (5 mL) was added TFA (1.0 mL, 13 mmol), and the resulting reaction was allowed to stir at 0° C. for 1 hour. The reaction mixture was filtered, concentrated in vacuo, and lyophilized to provide the 3-amino-1-(2-aminoethyl)pyrrolidin-2-one v. LCMS (ESI) m/z: 144.1 [M+H]⁺.

### Step E—Synthesis of Compound vi

To a solution of compound v (200 mg, 1.40 mmol) in DCM (10 mL) was added TEA (0.389 mL, 2.79 mmol), followed by a solution of Boc₂O (0.259 mL, 1.12 mmol) in DCM (10 mL) at 0° C., and the resulting reaction was allowed to stir at 0° C. for 4 hours. The reaction mixture was concentrated in vacuo to provide the crude compound tert-butyl (2-(3-amino-2-oxopyrrolidin-1-yl)ethyl)carbamate vi which was used without further purification. LCMS (ESI) m/z: 244.4 [M+H]⁺.

### Example 2

Preparation of Intermediate Compounds ix-xvi

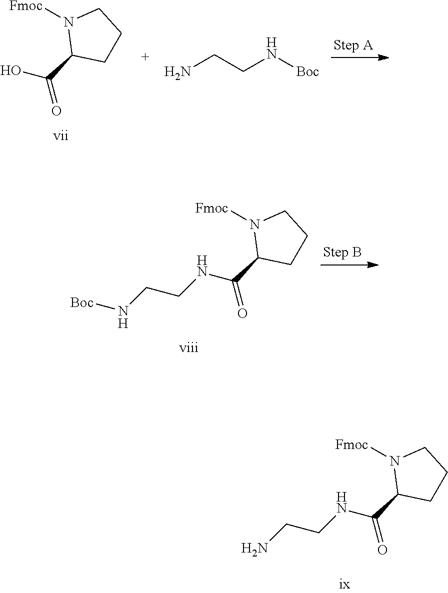

Step A—Synthesis of Compound viii

To a solution of (((9H-fluoren-9-yl)methoxy)carbonyl)-L-proline (vii, 2.0 g, 5.9 mmol), 1H-benzo[d][1,2,3]triazol-1-ol (0.801 g, 5.93 mmol) in DCM (40 mL) was added tert-butyl (2-aminoethyl)carbamate (1.05 g, 6.52 mmol) in DCM (3 mL) and EDC (1.25 g, 6.52 mmol) at 0° C. and the resulting reaction was allowed to warm to room temperature and stir at 25° C. for 16 hours. Saturated aqueous sodium hydrogen carbonate (50 mL) was added and the mixture was extracted with DCM (50 mL×2). The combined organic extracts were washed with aqueous HCl (0.1 M, 50 mL), dried over Na₂SO₄, filtered, and the solvent was evaporated under reduced pressure. The residue obtained was purified using flash silica gel chromatography (0 to 100% EtOAc/Petroleum ether) to provide (9H-fluoren-9-yl)methyl (S)-2-((2-((tert butoxycarbonyl)amino)ethyl)carbamoyl)pyrrolidine-1-carboxylate (viii, 2.5 g, 5.21 mmol). MS (ESI): m/z [M+H]⁺480.3

### Step B—Synthesis of Compound ix

To a solution of compound viii (500 mg, 1.0 mmol) in DCM (6 mL) was added TFA (2.0 mL, 26.0 mmol) and the resulting reaction was allowed to stir at 0° C. for 30 minutes. The reaction mixture was then concentrated in vacuo to provide 9H-fluoren-9-yl)methyl (S)-2-((2-aminoethyl)carbamoyl)pyrrolidine-1-carboxylate, ix, which was used without further purification. MS (ESI) m/z: 380.3 [M+H]⁺

The following intermediate compounds of the present disclosure were made using the methods described in Example 2, and substituting the appropriate reactants and/or reagents:

| | | MS |
|---|---|---|
| Compound | Structure | [M + H] |
| x | 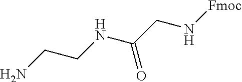 | 340 |
| xi | 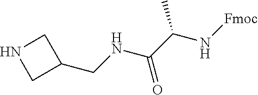 | 380 |
| xii | 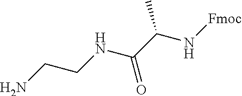 | 354 |
| xiii | 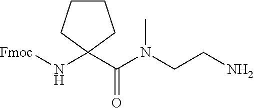 | 408 |
| xiv | 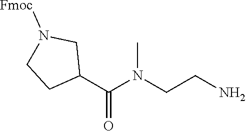 | 394 |
| xv | 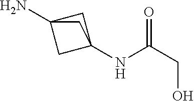 [SMILES: NC12CC(NC(=O)CO)(C1)C2] | 157 |
| xvi | 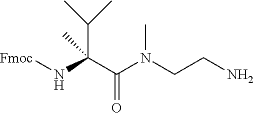 | 410 |

### Example 3

Preparation of Intermediate Compounds xx-xxii

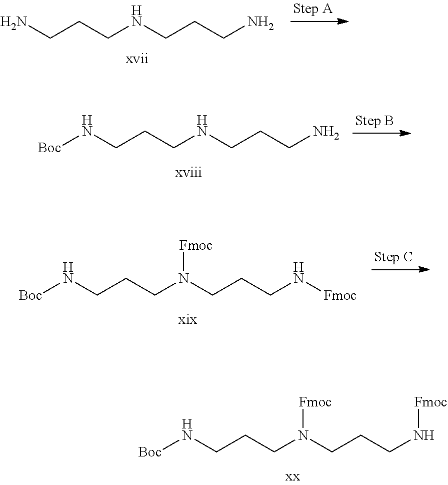

Step A—Synthesis of Compound xviii

To a solution of N1-(3-aminopropyl)propane-1,3-diamine (xvii, 5 g, 38.1 mmol) in THF (200 mL) was added tert-butyl 1H-imidazole-1-carboxylate (9.61 g, 57.2 mmol), and the resulting reaction was allowed to stir at 70° C. for 16 hours. The reaction mixture was then cooled to 20° C., and concentrated in vacuo, to provide tert-butyl (3-((3-aminopropyl)amino)propyl)carbamate-di-tert-butyl (azanediylbis(propane-3,1-diyl))dicarbamate (xviii, 15 g, 23.99 mmol) as an oil, which was used without further purification. LCMS (ESI) m/z: 232.1 [M+H]⁺.

### Step B—Synthesis of Compound xix

To a solution of compound xviii (15 g, 26.7 mmol) in THF (150 ML) was added K₂CO₃ (11.1 g. 80.0 mmol) in H₂O (150 mL), and the reaction mixture was allowed to stir at 20° C. for 17 hours. The reaction mixture was then diluted with EtOAc (200 mL), washed with H₂O (50 mL) and brine (50 mL), dried over Na₂SO₄, filtered, and concentrated in vacuo. The residue obtained was purified using flash silica gel chromatography (0-50% EtOAc/Petroleum) to provide the compound (9H-fluoren-9-yl)methyl (3-((((9H-fluoren-9-yl)methoxy)carbonyl)amino)propyl)(3-((tert-butoxycarbonyl)amino)propyl)carbamate (xix, 9.5 g. 13.35 mmol) as an oil. LCMS (ESI) m/z: 676.3 [M+H]⁺.

### Step C—Synthesis of Compound xx

A solution of compound xix (1.50 g, 2.22 mmol) and TFA (10 mL, 2.22 mmol) in DCM (50 mL) was allowed to stir at 20° C. for 18 hours. The reaction mixture was then concentrated in vacuo to provide (9H-fluoren-9-yl)methyl (3-((((9H-fluoren-9-yl)methoxy)carbonyl)amino)propyl)(3-aminopropyl)carbamate (xx, 1.5 g, 2.0 mmol) as an oil, which was used without further purification. LCMS (ESI) m/z: 576.3 [M+H]⁺. ¹H NMR (400 MHz, CD₃OD) δ 7.70-7.82 (m, 4H), 7.52-7.64 (m, 4H), 7.24-7.40 (m, 8H), 4.57-4.72 (m, 2H), 4.29-4.41 (m, 2H), 4.07-4.26 (m. 2H), 3.08-3.28 (m, 2H), 2.84-3.01 (m. 2H), 2.75 (br s, 2H). 2.42 (br s, 1H), 1.75 (br s, 1H), 1.31 (br s, 1H).

The following intermediate compounds of the present disclosure were made using the methods described in Example 3, and substituting the appropriate reactants and/or reagents:

| | | MS |
|---|---|---|
| | | [M + |
| Compound | Structure | H] |
| xxi | 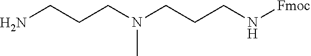 | 368 |
| xxii | 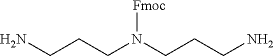 | 354 |

### Example 4

Preparation of Intermediate Compounds xxiii-xxvii

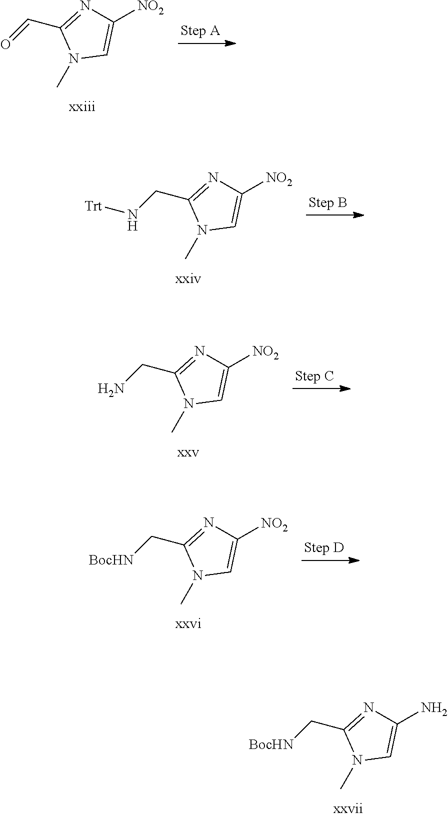

Step A—Synthesis of Compound xxiv

To a solution of compound xxiii (4.67 g, 30.1 mmol) in MeOH (20 mL) was added triphenylmethanamine (10.2 g, 39.1 mmol), acetic acid (0.18 g, 3.0 mmol) and sodium triacetoxyborohydride (9.57 g, 45.2 mmol), the mixture was stirred at 50° C. for 18 hours. Then sodium cyanoborohydride (3.78 g, 60.2 mmol) was added and the reaction mixture was stirred at 50° C. for 18 hours. The reaction mixture was then diluted with water (25 mL) and extracted with ethyl acetate (3×30 mL). The combined layers were washed with saturated aqueous sodium chloride (2×20 mL), dried over anhydrous sodium sulfate and concentrated in vacuo to provide N-((1-methyl-4-nitro-1H-imidazol-2-yl)methyl)-1,1,1-triphenylmethanamine xxiv (8 g, 6.02 mmol) as a solid. LCMS (ESI) m/z: 421.3 [M+Na]^{|}.

### Step B—Synthesis of Compound xxv

To a solution of xxiv (5.00 g, 12.6 mmol) in DCM (20 mL) was added TFA (4.0 mL, 52 mmol) and the mixture was stirred at 25° C. for 1 hour. The mixture was concentrated in vacuo to provide (1-methyl-4-nitro-1H-imidazol-2-yl)methanamine xxv (2.6 g, 10 mmol) as an oil, which was used without further purification.

Step C—Synthesis of Compound xxvi

To a solution of xxvi (2.6 g, 10 mmol) in DCM (30 mL) was added triethylamine (4.18 mL, 30.0 mmol), followed by di-tert-butyl dicarbonate (4.64 mL, 20.0 mmol) dropwise slowly. The solution was stirred at 20° C. for 18 hours. Then the reaction mixture was purified using silica gel column chromatography (60%-90% EtOAc/petroleum ether) to provide tert-butyl ((1-methyl-4-nitro-1H-imidazol-2-yl)methyl)carbamate xxvi (850 mg, 2.99 mmol) as a solid. LCMS (ESI) m/z: 257.2 [M+H]*.

Step D—Synthesis of Compound xxvii

To a mixture of xxvi (230 mg, 0.898 mmol) in trifluoroethanol (3 mL) was added 10% palladium on carbon (96 mg. 0.090 mmol) and the mixture was stirred at 20° C. for 0.5 hours under a H₂ atmosphere (15 psi). The mixture was filtered through a short Celite®, plug and the filtrate was concentrated in vacuo to provide tert-butyl ((4-amino-1-methyl-1H-imidazol-2-yl)methyl)carbamate xxvii (200 mg, 0.619 mmol) as a yellow solid, which was used without further purification. LCMS (ESI) m/z: 227.0 [M+H]+.

### Example 5

Preparation of Intermediate Compounds xxviii-xxx

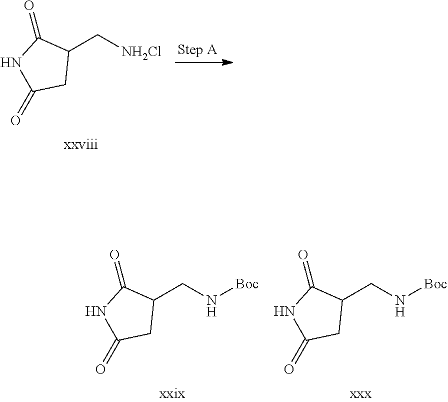

Step A—Synthesis of Compounds xxix and xxx

To a solution of xxviii (200 mg, 1.22 mmol) in DCM (3 mL) was added triethylamine (0.169 mL, 1.22 mmol) and di-tert-butyl dicarbonate (0.423 mL, 1.82 mmol), the reaction solution was stirred at 20° C. for 2 hours. The mixture was concentrated in vacuo and the residue was purified by preparative SFC (DAICEL CHIRALPAK IC (250 mm×30 mm×10 um), eluting with 20% 0.10% NH₃H₂O EtOH/acetonitrile at a flow rate of 70 mL/min) to provide xxix (53 mg, 0.232 mmol) and xxx (58 mg, 0.254 mmol) as solids. The material was used directly in the next step. LCMS (ESI) m/z: 251.1 [M+H]⁺.

### Example 6

Preparation of Intermediate Compounds xxxii-xxxiii

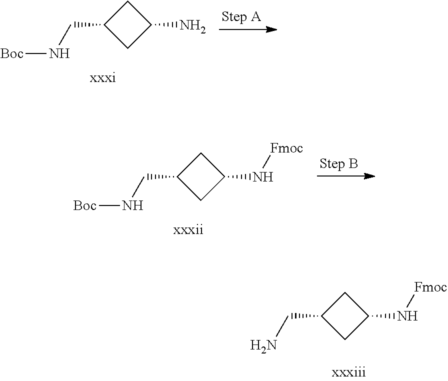

Step A—Synthesis of Compound xxxii

To a stirred solution of tert-butyl (((1s,3s)-3-aminocyclobutyl)methyl)carbamate (xxxi, 150 mg, 0.749 mmol) in DCM (10 mL) was added DIPEA (0.392 mL, 2.25 mmol). The resulting solution was allowed to stir at 20° C. for 10 minutes, then N-(9-fluorenylmethoxycarbonyl)succinimide (303 mg, 0.899 mmol) was added. The resulting reaction was allowed to stir at 20° C. for 18 hours, then concentrated in vacuo. The residue obtained was purified using flash silica gel chromatography (60-70% EtOAc/petroleum ether) to provide tert-butyl (((1s,3s)-3-((((9H-fluoren-9-yl)methoxy)carbonyl)amino)cyclobutyl)methyl) carbamate xxxii (180 mg, 0.405 mmol). MS (ESI) m/z: 445.3 [M+Na]⁺.

Step B—Synthesis of Compound xxxiii

To a stirred solution of compound xxxii (170 mg, 0.402 mmol) in DCM (0.9 mL) was added TFA (0.300 mL, 3.89 mmol), and the resulting reaction was allowed to stir at 25° C. for 2 hours. The reaction mixture was then concentrated in vacuo to provide (9H-fluoren-9-yl)methyl ((1s,3s)-3-(aminomethyl)cyclobutyl) carbamate xxxiii, which was used without further purification. MS (ESI) m/z: 323.1 [M+H]⁺.

The following intermediate compounds of the present disclosure were made using the methods described in Example 6, and substituting the appropriate reactants and/or reagents:

| | | MS |
|---|---|---|
| Compound | Structure | [M + H] |
| xxxiv | 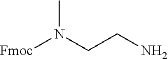 | 297 |
| xxxv | 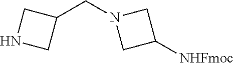 | 364 |
| xxxvi | 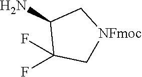 | 345 |
| xxxvii | 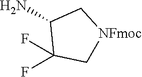 | 345 |
| xxxviii | 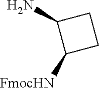 | 309 |
| xxxix | 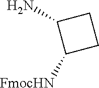 | 309 |
| xl | 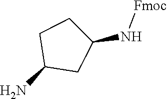 | 323 |
| xli | 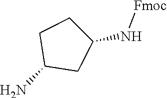 | 323 |
| xlii | 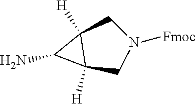 | 321 |
| xliii | 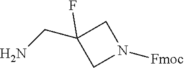 | 327 |
| xliv | 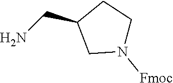 | 323 |
| xlv | 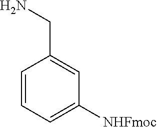 | 345 |
| xlvi | 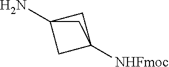 | 321 |
| xlvii | 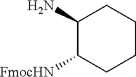 | 337 |
| xlviii | 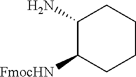 | 337 |
| xlix | 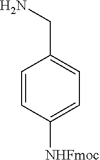 | 345 |
| l | 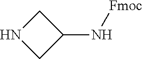 | 295 |
| li | 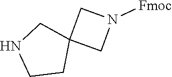 | 335 |
| lii | 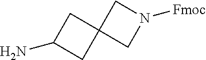 | 335 |
| liii | 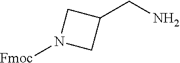 | 309 |
| liv | 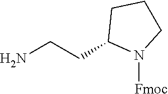 | 337 |
| lv | 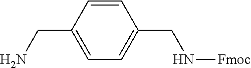 | 359 |
| lvi | 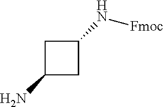 | 309 |
| lvii | 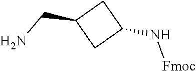 | 323 |
| lviii | 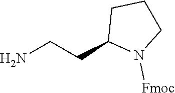 | 337 |
| lix | 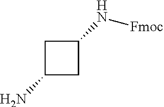 | Used directly in next step |
| lx | 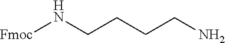 | 311 |
| lxi | 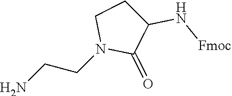 | 366 |
| lxii | 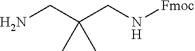 | 325 |
| lxiii | 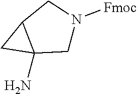 | 321 |
| lxiv | 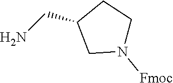 | 323 |
| lxv | 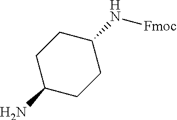 | 337 |
| lxvi | 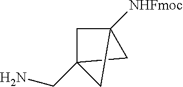 | 335 |
| lxvii | 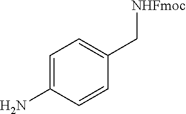 | 345 |
| lxviii | 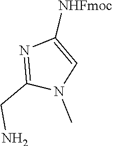 | 349 |

### Example 7

### Preparation of Intermediate Compounds lxx

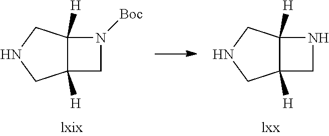 [SMILES: CC(C)(C)OC(=O)N1C[C@@H]2CNC[C@@H]21 ; C1NC[C@@H]2NC[C@H]12]

To a stirred solution of tert-butyl (1S,5R)-3,6-diazabicyclo[13.2.0]heptane-6-carboxylate (Ixix, 200 mg, 1.01 mmol) in DCM (1.5 mL) was added TFA (0.500 mL, 6.49 mmol), and the resulting reaction was allowed to stir at 25° C. for 2 hours. The reaction mixture was concentrated in vacuo to provide (1S,5R)-3,6-diazabicyclo[3.2.0]heptane (lxx), which was used without further purification.

The following intermediate compounds of the present disclosure were made using the methods described in Example 7, and substituting the appropriate reactants and/or reagents:

| | | MS |
|---|---|---|
| Compound | Structure | [M + H] |
| lxxi | 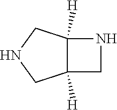 | NA |
| lxxii | 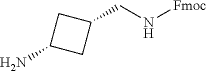 | 323 |
| lxxiii | 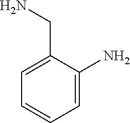 | NA |
| lxxiv | 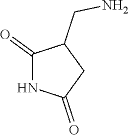 | NA |
| lxxv | 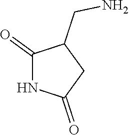 | NA |

### Example 8

Preparation of Intermediate Compound lxxxi

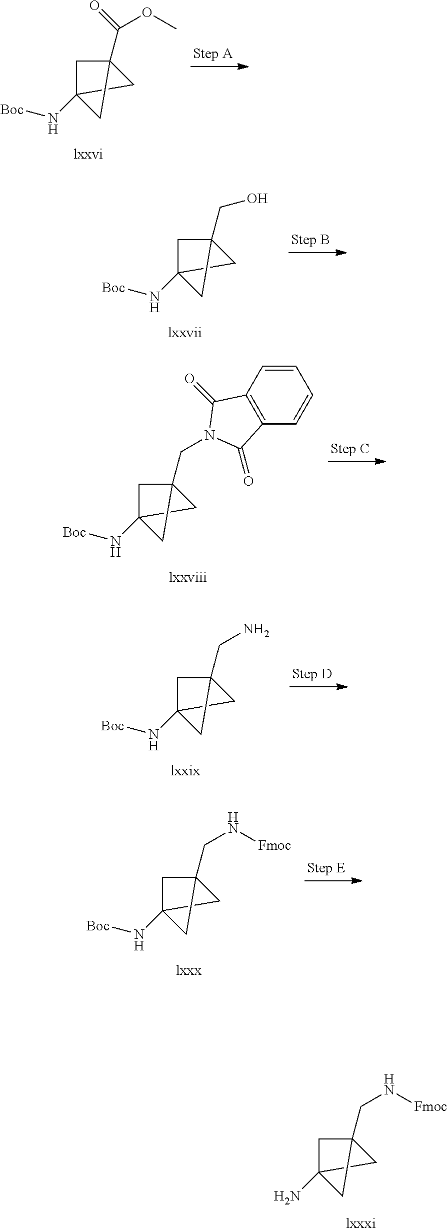

Step A—Synthesis of Compound lxxvii

To a solution of compound lxxvi (4.0 g, 17 mmol) in THF (20 mL)) was added LAH (1.0 M in THF. 24.9 mL, 24.9 mmol) at 0° C. under N₂. The resulting reaction was allowed to stir at 0° C. for 1 hour. The following were then sequentially added to the reaction mixture: 1 mL water, 1 mL aq.15% NaOH, and 3 mL water. The resulting suspension was filtered, washed with MeOH (50 mL) and the filtrate was concentrated in vacuo to provide tert-butyl (3-(hydroxymethyl)bicyclo[1.1.1]pentan-1-yl)carbamate (lxxvi, 2.50 g, 10.6 mmol) which was used without further purification.

Step B—Synthesis of Compound lxxviii

To a solution of compound lxxvi (1.50 g, 7.03 mmol) in THF (50 mL) was added isoindoline-1,3-dione (1.24 g, 8.44 mmol) and triphenylphosphine (2.77 mg, 10.6 mmol) followed by di-tert-butyl diazene-1,2-dicarboxylate (2.43 mg. 10.6 mmol), and the resulting reaction was allowed to stir at 20° C. for 17 hours. The reaction mixture was diluted with EtOAc (300 mL), washed with water (50 mL) and brine (50 mL), dried over Na₂SO₄, filtered and concentrated in vacuo. The resulting residue was purified using preparative HPLC (Boston Green ODS 150×30 mm×5 μm, 30% to 50% ACN/Water (with 0.1% TFA as modifier)) to provide tert-butyl (3-((1,3-dioxoisoindolin-2-yl)methyl)bicyclo[1.1.1]pentan-1-yl)carbamate (lxxviii, 1.50 g, 3.94 mmol). LCMS (ESI) m/z: 287.1 [(M−56)+H]⁺.

Step C—Synthesis of Compound lxxix

To a solution of compound lxxviii (1.00 g, 2.92 mmol) in EtOH (30 mL) was added hydrazine hydrate (1.72 g, 29.2 mmol), and the resulting reaction was allowed to stir at 20° C. for 17 hours. The reaction mixture was filtered, and the filtrate lyophilized to provide tert-butyl (3-(aminomethyl)bicyclo[1.1.1]pentan-1-yl)carbamate (lxxix, 700 mg, 2.64 mmol) which was used without further purification. LCMS (ESI) m/z: 213.1[M+H]⁺.

Step D—Synthesis of Compound lxxx

To a solution of compound lxxix (200 mg, 0.942 mmol) in DCM (10 mL) was added DIEA (0.329 mL, 1.88 mmol) followed by 9-fluorenylmethyl chloroformate (366 mg, 1.41 mmol), and the resulting reaction was allowed to stir at 20° C. for 17 hours. The reaction mixture was concentrated in vacuo, and the mixture was purified using preparative HPLC (Boston Green ODS 150×30 mm×5 μm, 20% to 40% ACN/Water (with 0.1% TFA as modifier)) to provide tert-butyl (3-(((((9H-fluoren-9-yl)methoxy)carbonyl)amino)methyl) bicyclo[1.1.1]pentan-1-yl)carbamate (lxxx, 250 mg, 0.518 mmol). LCMS (ESI) m/z: 457.2 [M+Na]⁺.

Step E—Synthesis of Compound lxxxi

To a solution of compound lxxx (240 mg, 0.552 mmol) in DCM (1.5 mL) was added TFA (0.5 mL) and the resulting reaction was allowed to stir at 20° C. for 1 hour. The reaction mixture was then concentrated in vacuo to provide (9H-fluoren-9-yl)methyl ((3-aminobicyclo[1.1.1]pentan-1-yl)methyl)carbamate (lxxxi. 235 mg, 0.512 mmol) which was used without further purification. LCMS (ESI) m/z: 335.2 [M+H]⁺.

### Example 9

Preparation of Intermediate Compound lxxxiii

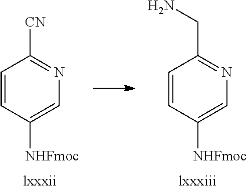

To a solution of (9H-fluoren-9-yl)methyl (6-cyanopyridin-3-yl)carbamate (lxxxii, 1.2 g, 3.5 mmol) in THF (10 mL) was added borane in THF (1.0 M, 14.1 mL, 14.1 mmol) at 25° C. under argon atmosphere. The resulting reaction was warmed to 70° C., and allowed to stir at this temperature for 6 hours. The reaction mixture was quenched using acetic acid (5 mL), and the resulting mixture was warmed to 70° C., and allowed to stir at this temperature for 16 hours. The reaction mixture was concentrated in vacuo, and the resulting residue was purified using silica gel column chromatography, eluting with 1 to 10% methanol in DCM to provide (9H-fluoren-9-yl)methyl(6-(aminomethyl)pyridin-3-yl)carbamate (lxxxiii, 500 mg, 1.45 mmol). MS: m/z=346.25 [M+H]⁺.

### Example 10

Preparation of Intermediate Compound lxxxix

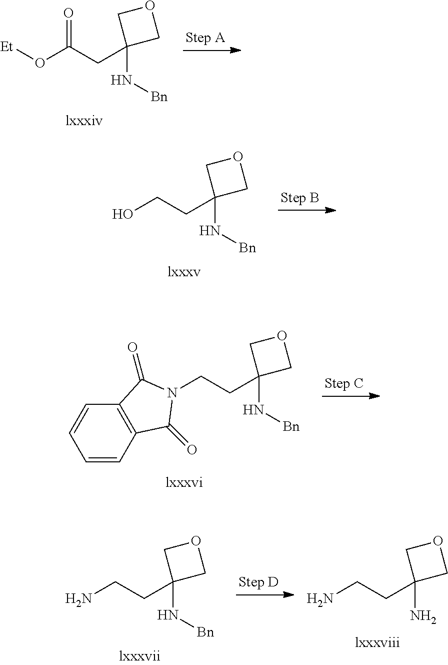 [SMILES: CCOC(=O)CC1(NCc2ccccc2)COC1 ; OCCC1(NCc2ccccc2)COC1 ; O=C1c2ccccc2C(=O)N1CCC1(NCc2ccccc2)COC1 ; NCCC1(NCc2ccccc2)COC1 ; NCCC1(N)COC1]

Step A—Synthesis of Compound lxxxv

To a solution of ethyl 2-(3-(benzylamino)oxetan-3-yl)acetate (lxxxiv, 1.50 g, 6.02 mmol) in THF (5 mL)) was added LAH (457 mg, 12.0 mmol) at 0° C. under N₂. The resulting reaction was allowed to stir at 0° C. for 1 hour, then H₂O (0.5 mL), 15% NaOH (0.5 mL, aq.) and H₂O (1.5 mL), were added sequentially. The resulting suspension was filtered, washed with DCM (20 mL), then concentrated in vacuo to provide 2-(3-(benzylamino)oxetan-3-yl)ethan-1-ol (lxxxv, 1.1 g. 4.25 mmol) as an oil, which was used without further purification. LCMS (ESI) m/z: 208.1 [M+H]⁺.

Step B—Synthesis of Compound lxxxvi

To a solution of compound lxxxv (1.00 g, 4.82 mmol) in THF (30 mL) was added isoindoline-1,3-dione (710 mg, 4.82 mmol) and triphenylphosphine (1.27 g, 4.82 mmol), followed by di-tert-butyl diazene-1,2-dicarboxylate (1.11 g. 4.82 mmol). The resulting reaction was allowed to stir at 20° C. for 17 hours, then the reaction mixture was diluted with EtOAc (100 mL). The resulting solution was washed sequentially with H₂O (50 mL) and brine (50 mL), dried over Na₂SO₄, filtered, and concentrated in vacuo. The residue obtained was purified using silica gel chromatography (0-50% EtOAc/Petroleum) to provide 2-(2-(3-(benzylamino)oxetan-3-yl)ethyl)isoindoline-1,3-dione (lxxxvi, 1.00 g, 2.68 mmol) as an oil. LCMS (ESI) m/z: 337.2 [M+H]⁺.

Step C—Synthesis of Compound lxxxvii

To a solution of compound lxxxvi (1.00 g, 2.97 mmol) in MeOH (10 mL) was added hydrazinium hydroxide (558 mg, 8.92 mmol), and the resulting reaction was allowed to stir at 20° C. for 17 hours. The reaction mixture was filtered, and directly purified using prep-HPLC (YMC-Actus Triart C18 150×30 mm×5 um, eluted with MeCN/water (0.1% TFA) from 0 to 60%), then lyophilized to provide 3-(2-aminoethyl)-N-benzyloxetan-3-amine (lxxxvii, 400 mg, 1.745 mmol) as an oil. LCMS (ESI) m/z: 207.1 [M+H]⁺.

Step D—Synthesis of Compound lxxxviii

To a solution of compound lxxxvii (400 mg, 1.94 mmol) in CF₃CH₂OH (5 mL) was added Pd—C (103 mg, 0.0970 mmol) under N₂. The resulting reaction was purged with H₂ three times, and the reaction was allowed to stir at 20° C. for 9 hours under H₂ atmosphere (provided by using an H₂ filled balloon). The reaction mixture was then filtered, and concentrated in vacuo to provide 3-(2-aminoethyl)oxetan-3-amine (lxxxviii, 250 mg, 1.93 mmol) as an oil which was used without further purification. LCMS (ESI) m/z: 134.1 [M+H₂O].

### Example 11

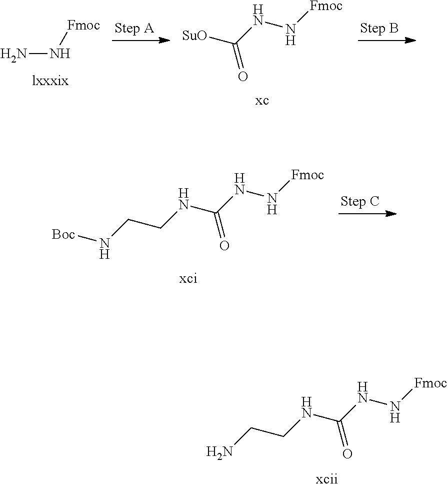

### Step A—Synthesis of Compound xc

To a solution of (9H-fluoren-9-yl)methyl hydrazinecarboxylate (lxxxix, 3.00 g, 11.8 mmol) in DMF (50.0 mL) was added bis(2,5-dioxopyrrolidin-1-yl) carbonate (3.02 g, 11.8 mmol), and the resulting reaction was allowed to stir at 25° C. for 1 hour. The reaction mixture, which contained 1-((9H-fluoren-9-yl)methyl) 2-(2,5-dioxopyrrolidin-1-yl) hydrazine-1,2-dicarboxylate (xc, 4.66 g, 11.8 mmol) was used directly in the next step without further purification.

### Step B—Synthesis of Compound xci

To a solution of compound xc (4.66 g, 11.8 mmol) in DMF (50.0 ml) was added tert-butyl (2-aminoethyl)carbamate (1.89 g, 11.8 mmol), and the resulting reaction was allowed to stir at 25° C. for 16 hours. Water (50.0 mL) was added, and the resulting mixture was extracted with ethyl acetate (2×40.0 mL). The combined organic extracts were washed with brine (50.0 mL), dried over Na₂SO₄, filtered, and concentrated in vacuo. The residue obtained was purified using flash silica gel chromatography (5% MeOH/DCM) to provide (9H-fluoren-9-yl)methyl 11,11-dimethyl-4,9-dioxo-10-oxa-2,3,5,8-tetraazadodecanoate (xci, 3.7 g, 8.40 mmol) as a solid. LCMS: MS (ESI) m/z: 441.3 [M+H]⁺.

Step C—Synthesis of Compound xcii

A solution of compound xci (1.00 g, 2.27 mmol) in TFA (3.00 ml) and DCM (10.0 mL) was allowed to stir at 25° C. for 1 hour. The reaction mixture was then concentrated in vacuo to provide (9H-fluoren-9-yl)methyl 2-((2-aminoethyl)carbamoyl)hydrazine-1-carboxylate (xcii, 770 mg, 2.262 mmol) as an oil, which was used without further purification. LCMS: MS (ESI) m/z: 341.1 [M+H]⁺.

### Example 12

Preparation of Intermediate Compound xciv

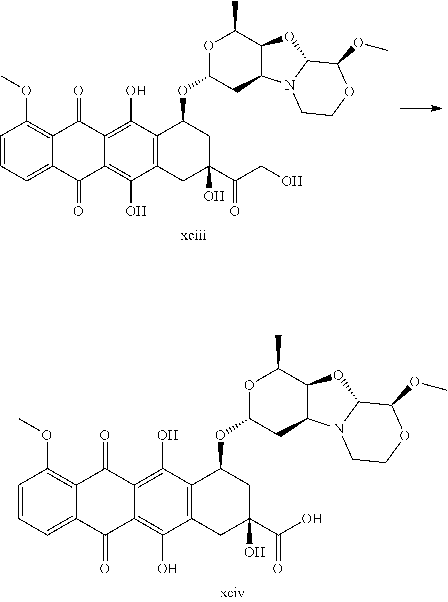 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC)OCCN32)[C@H](C)O1 ; COc1cccc2c1C(=O)c1c(O)c3c(c(O)c1C2=O)C[C@@](O)(C(=O)O)C[C@@H]3O[C@H]1C[C@H]2[C@H](O[C@@H]3[C@@H](OC)OCCN32)[C@H](C)O1]

To a solution of (8S,10S)-6,8,11-trihydroxy-8-(2-hydroxyacetyl)-1-methoxy-10-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-7,8,9,10-tetrahydrotetracene-5,12-dione (xciii, 200 mg, 0.312 mmol) in methanol (5 mL) and water (5 mL) was added a solution of sodium periodate (80 mg, 0.37 mmol) in water (1 mL). The resulting reaction was allowed to stir at room temperature for 30 minutes, then the solvent was gradually removed from the reaction mixture in vacuo at 25° C. over 18 hours. The resulting product xciv was used without further purification. LCMS (ESI) m/z: 628.6 [M+H]⁺.

### Preparation of Illustrative Compounds

Examples 13-14 describe the synthesis of illustrative compounds of the present disclosure.

### Example 13

### Preparation of Compounds 1-60

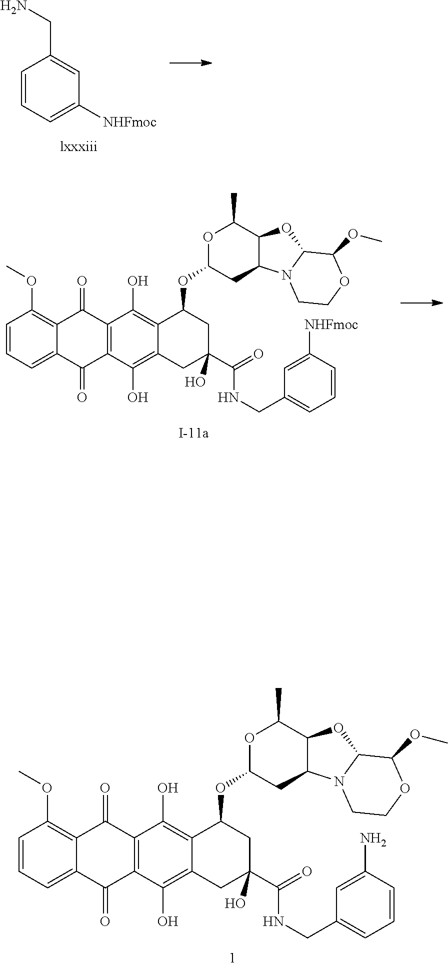

### Step A—Synthesis of Compound I-11a

To a solution of compound lxxxiii (200 mg, 0.319 mmol) in DMF (5 mL) was added HATU (242 mg, 0.637 mmol). (9H-fluoren-9-yl)methyl (3-(aminomethyl)phenyl)carbamate (132 mg, 0.382 mmol) and N-ethyl-N-isopropylpropan-2-amine (0.158 mL, 0.956 mmol) at room temperature under argon atmosphere. The resulting reaction was allowed to stir at room temperature for 2 hours. The reaction mixture was then directly purified using reverse phase Combiflash (Column: C18 Column, 40 g, 60 Å, 20-35 μm; 0% to 100% ACN/water) to provide (9H-fluoren-9-yl)methyl(3-(((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)methyl)phenyl)carbamate (I-11a, 150 mg. 0.157 mmol). LCMS MS (ESI): m/z=954.55 [M+H]⁺.

### Step B—Synthesis of Compound 1

To a solution of compound 1-11a (150 mg, 0.157 mmol) in DMF (6 mL) at 0° C. was added piperidine (0.20 mL, 0.16 mmol) under argon atmosphere. The resulting reaction was allowed to stir at 0° C. for 20 minutes, then the reaction mixture was concentrated in vacuo. The residue obtained was purified using reverse phase Combiflash (Column: C18 Column, 40 g, 60 Å, 20-35 μm: 0% to 60% ACN/water) to provide (2S,4S)—N-(3-aminobenzyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (1, 56.7 mg. 0.077 mmol) as a solid. LCMS MS (ESI): m/z=732.40 [M+H]⁺. ¹H NMR (400 MHz, DMSO-d₆) δ 8.31 (t, J=2.8 Hz, 1H). 7.92-7.91 (m, 2H). 7.65 (t, J=3.2 Hz, 1H), 6.93 (t, J=7.6 Hz, 1H), 6.46-6.41 (m, 3H), 5.38 (s, 1H), 5.27-5.24 (m, 1H), 5.00 (s, 3H), 4.58 (s, 1H), 4.23-3.92 (m, 8H), 3.66-3.52 (m, 3H), 3.39-3.31 (m, 5H), 3.10-2.68 (m, 3H), 2.31-2.21 (m, 2H), 1.87-1.65 (m, 3H), 1.20 (d, J=6.4 Hz, 3H).

The following illustrative compounds of the present disclosure were made using the methods described in Example 13, and substituting the appropriate reactants and/or reagents:

| Com- | | MS |
|---|---|---|
| pound | Structure | [M + H] |
| 2 | 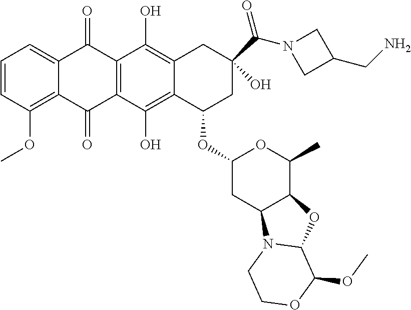 [SMILES: COc1cccc2c1C(=O)c1c(O)c3c(c(O)c1C2=O)C[C@@](O)(C(=O)N1CC(CN)C1)C[C@@H]3O[C@H]1C[C@H]2[C@H](O[C@@H]3[C@@H](OC)OCCN32)[C@H](C)O1] | 696 |
| 3 | 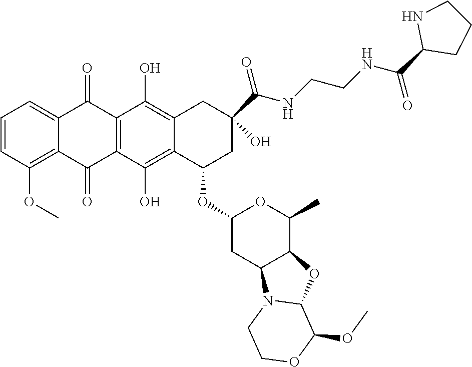 [SMILES: COc1cccc2c1C(=O)c1c(O)c3c(c(O)c1C2=O)C[C@@](O)(C(=O)NCCNC(=O)[C@@H]1CCCN1)C[C@@H]3O[C@H]1C[C@H]2[C@H](O[C@@H]3[C@@H](OC)OCCN32)[C@H](C)O1] | 767 |
| 4 | 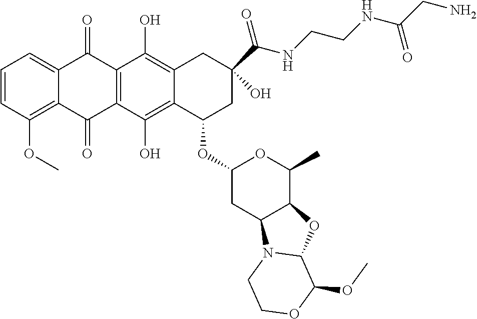 [SMILES: COc1cccc2c1C(=O)c1c(O)c3c(c(O)c1C2=O)C[C@@](O)(C(=O)NCCNC(=O)CN)C[C@@H]3O[C@H]1C[C@H]2[C@H](O[C@@H]3[C@@H](OC)OCCN32)[C@H](C)O1] | 727 |
| 5 | 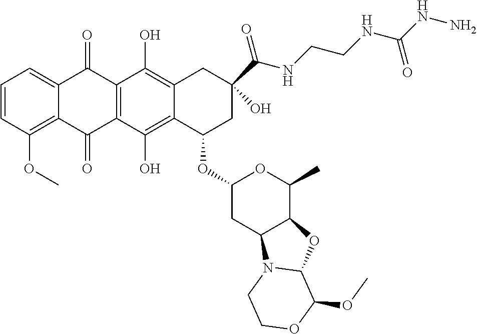 [SMILES: COc1cccc2c1C(=O)c1c(O)c3c(c(O)c1C2=O)C[C@@](O)(C(=O)NCCNC(=O)NN)C[C@@H]3O[C@H]1C[C@H]2[C@H](O[C@@H]3[C@@H](OC)OCCN32)[C@H](C)O1] | 728 |
| 6 | 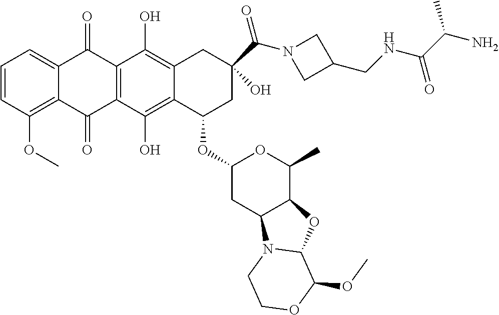 [SMILES: COc1cccc2c1C(=O)c1c(O)c3c(c(O)c1C2=O)C[C@@](O)(C(=O)N1CC(CNC(=O)[C@H](C)N)C1)C[C@@H]3O[C@H]1C[C@H]2[C@H](O[C@@H]3[C@@H](OC)OCCN32)[C@H](C)O1] | 767 |
| 7 | 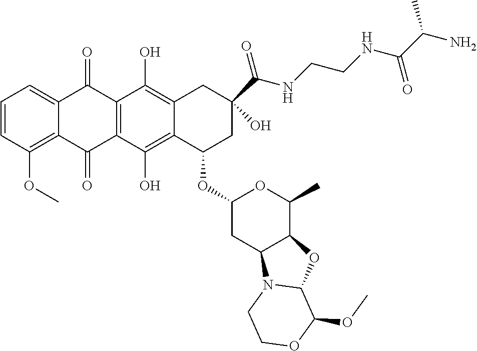 [SMILES: COc1cccc2c1C(=O)c1c(O)c3c(c(O)c1C2=O)C[C@@](O)(C(=O)NCCNC(=O)[C@H](C)N)C[C@@H]3O[C@H]1C[C@H]2[C@H](O[C@@H]3[C@@H](OC)OCCN32)[C@H](C)O1] | 741 |
| 8 | 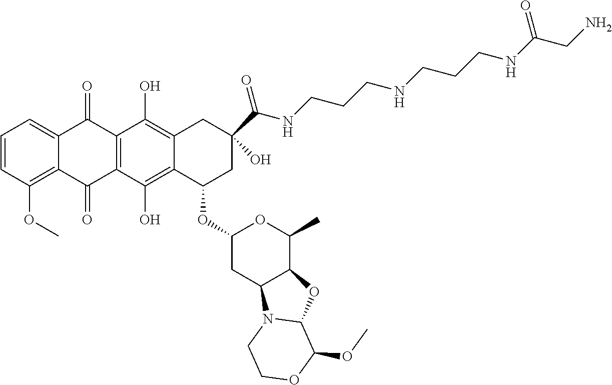 [SMILES: COc1cccc2c1C(=O)c1c(O)c3c(c(O)c1C2=O)C[C@@](O)(C(=O)NCCCNCCCNC(=O)CN)C[C@@H]3O[C@H]1C[C@H]2[C@H](O[C@@H]3[C@@H](OC)OCCN32)[C@H](C)O1] | 798 |
| 9 | 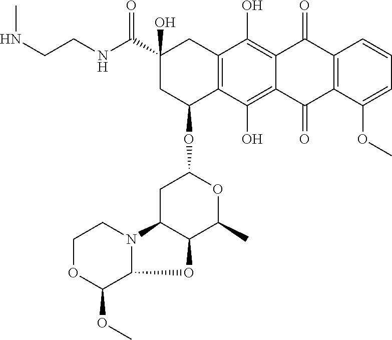 [SMILES: CNCCNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O] | 684 |
| 10 | 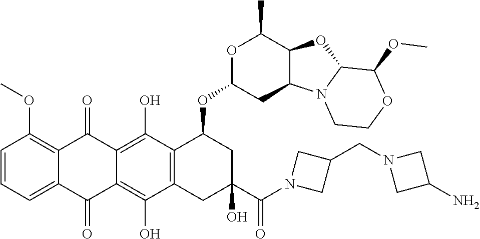 [SMILES: COc1cccc2c1C(=O)c1c(O)c3c(c(O)c1C2=O)C[C@@](O)(C(=O)N1CC(CN2CC(N)C2)C1)C[C@@H]3O[C@H]1C[C@H]2[C@H](O[C@@H]3[C@@H](OC)OCCN32)[C@H](C)O1] | 751 |
| 11 | 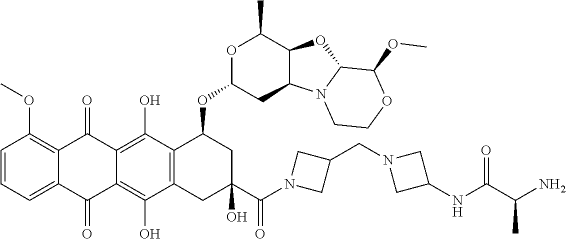 [SMILES: COc1cccc2c1C(=O)c1c(O)c3c(c(O)c1C2=O)C[C@@](O)(C(=O)N1CC(CN2CC(NC(=O)[C@H](C)N)C2)C1)C[C@@H]3O[C@H]1C[C@H]2[C@H](O[C@@H]3[C@@H](OC)OCCN32)[C@H](C)O1] | 822 |
| 12 | 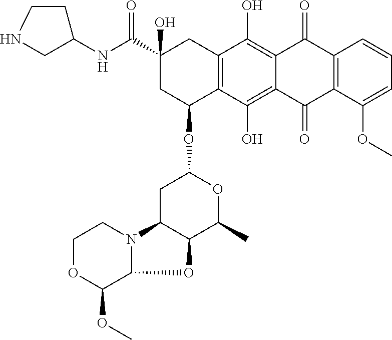 [SMILES: COc1cccc2c1C(=O)c1c(O)c3c(c(O)c1C2=O)C[C@@](O)(C(=O)NC1CCNC1)C[C@@H]3O[C@H]1C[C@H]2[C@H](O[C@@H]3[C@@H](OC)OCCN32)[C@H](C)O1] | 696 |
| 13 | 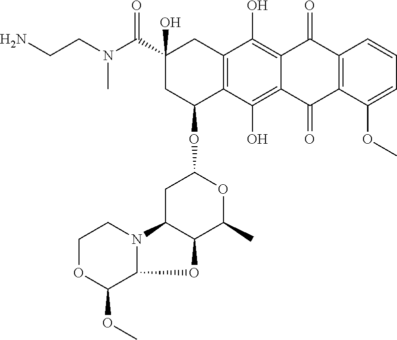 [SMILES: COc1cccc2c1C(=O)c1c(O)c3c(c(O)c1C2=O)C[C@@](O)(C(=O)N(C)CCN)C[C@@H]3O[C@H]1C[C@H]2[C@H](O[C@@H]3[C@@H](OC)OCCN32)[C@H](C)O1] | 684 |
| 14 | 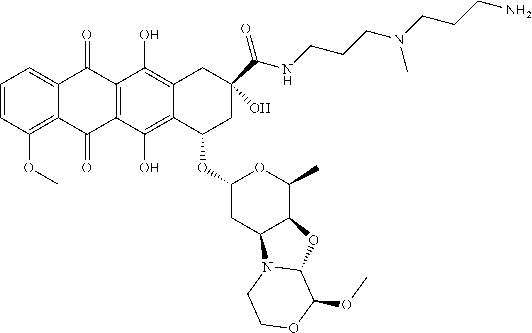 [SMILES: COc1cccc2c1C(=O)c1c(O)c3c(c(O)c1C2=O)C[C@@](O)(C(=O)NCCCN(C)CCCN)C[C@@H]3O[C@H]1C[C@H]2[C@H](O[C@@H]3[C@@H](OC)OCCN32)[C@H](C)O1] | 755 |
| 15 | 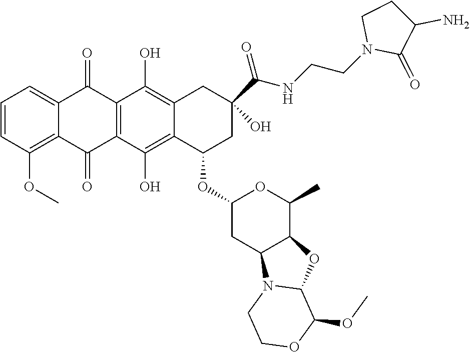 [SMILES: COc1cccc2c1C(=O)c1c(O)c3c(c(O)c1C2=O)C[C@@](O)(C(=O)NCCN1CCC(N)C1=O)C[C@@H]3O[C@H]1C[C@H]2[C@H](O[C@@H]3[C@@H](OC)OCCN32)[C@H](C)O1] | 753 |
| 16 | 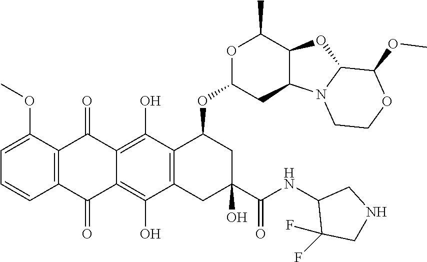 [SMILES: COc1cccc2c1C(=O)c1c(O)c3c(c(O)c1C2=O)C[C@@](O)(C(=O)NC1CNCC1(F)F)C[C@@H]3O[C@H]1C[C@H]2[C@H](O[C@@H]3[C@@H](OC)OCCN32)[C@H](C)O1] | 732 |
| 17 | 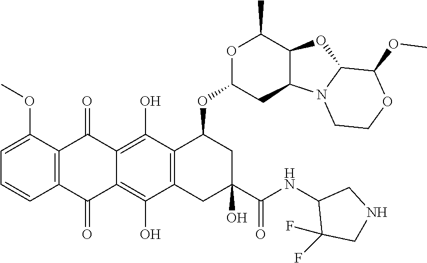 [SMILES: COc1cccc2c1C(=O)c1c(O)c3c(c(O)c1C2=O)C[C@@](O)(C(=O)NC1CNCC1(F)F)C[C@@H]3O[C@H]1C[C@H]2[C@H](O[C@@H]3[C@@H](OC)OCCN32)[C@H](C)O1] | 732 |
| 18 | 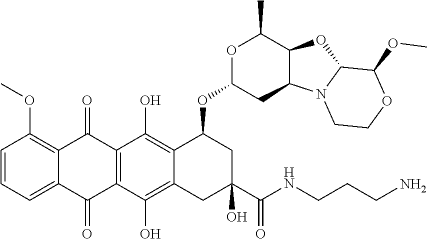 [SMILES: COc1cccc2c1C(=O)c1c(O)c3c(c(O)c1C2=O)C[C@@](O)(C(=O)NCCCN)C[C@@H]3O[C@H]1C[C@H]2[C@H](O[C@@H]3[C@@H](OC)OCCN32)[C@H](C)O1] | 684 |
| 19 | 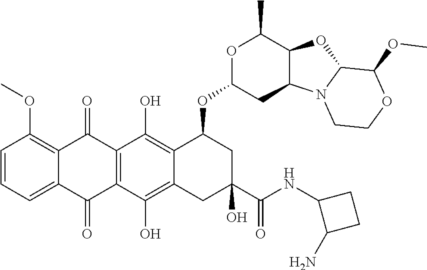 [SMILES: COc1cccc2c1C(=O)c1c(O)c3c(c(O)c1C2=O)C[C@@](O)(C(=O)NC1CCC1N)C[C@@H]3O[C@H]1C[C@H]2[C@H](O[C@@H]3[C@@H](OC)OCCN32)[C@H](C)O1] | 696 |
| 20 | 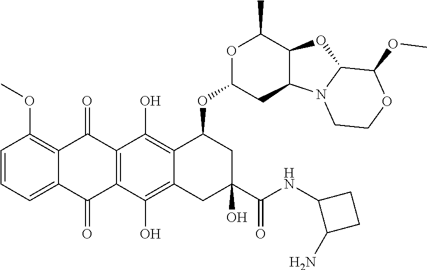 [SMILES: COc1cccc2c1C(=O)c1c(O)c3c(c(O)c1C2=O)C[C@@](O)(C(=O)NC1CCC1N)C[C@@H]3O[C@H]1C[C@H]2[C@H](O[C@@H]3[C@@H](OC)OCCN32)[C@H](C)O1] | 696 |
| 21 | 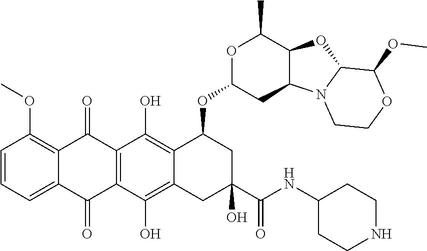 [SMILES: COc1cccc2c1C(=O)c1c(O)c3c(c(O)c1C2=O)C[C@@](O)(C(=O)NC1CCNCC1)C[C@@H]3O[C@H]1C[C@H]2[C@H](O[C@@H]3[C@@H](OC)OCCN32)[C@H](C)O1] | 710 |
| 22 | 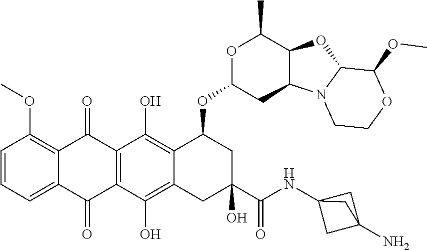 [SMILES: COc1cccc2c1C(=O)c1c(O)c3c(c(O)c1C2=O)C[C@@](O)(C(=O)NC12CC(N)(C1)C2)C[C@@H]3O[C@H]1C[C@H]2[C@H](O[C@@H]3[C@@H](OC)OCCN32)[C@H](C)O1] | 708 |
| 23 | 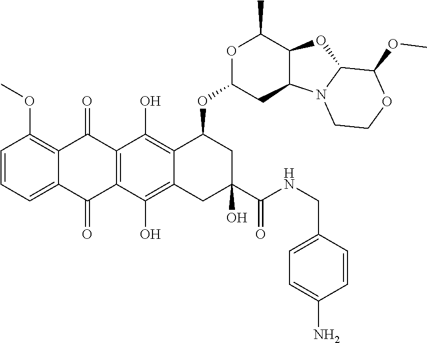 [SMILES: COc1cccc2c1C(=O)c1c(O)c3c(c(O)c1C2=O)C[C@@](O)(C(=O)NCc1ccc(N)cc1)C[C@@H]3O[C@H]1C[C@H]2[C@H](O[C@@H]3[C@@H](OC)OCCN32)[C@H](C)O1] | 732 |
| 24 | 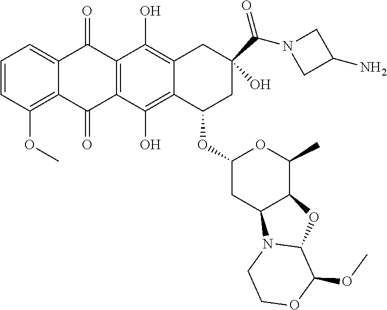 [SMILES: COc1cccc2c1C(=O)c1c(O)c3c(c(O)c1C2=O)C[C@@](O)(C(=O)N1CC(N)C1)C[C@@H]3O[C@H]1C[C@H]2[C@H](O[C@@H]3[C@@H](OC)OCCN32)[C@H](C)O1] | 682 |
| 25 | 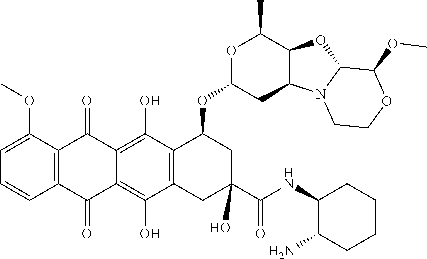 [SMILES: COc1cccc2c1C(=O)c1c(O)c3c(c(O)c1C2=O)C[C@@](O)(C(=O)N[C@H]1CCCC[C@@H]1N)C[C@@H]3O[C@H]1C[C@H]2[C@H](O[C@@H]3[C@@H](OC)OCCN32)[C@H](C)O1] | 724 |
| 26 | 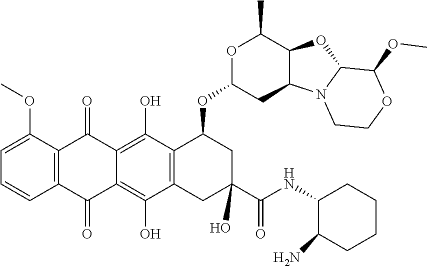 [SMILES: COc1cccc2c1C(=O)c1c(O)c3c(c(O)c1C2=O)C[C@@](O)(C(=O)N[C@@H]1CCCC[C@H]1N)C[C@@H]3O[C@H]1C[C@H]2[C@H](O[C@@H]3[C@@H](OC)OCCN32)[C@H](C)O1] | 724 |
| 27 | 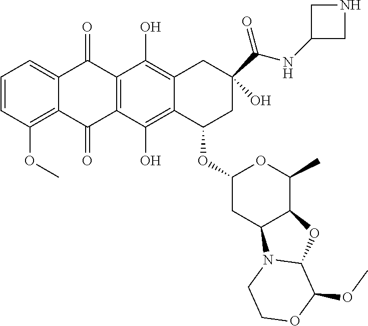 [SMILES: COc1cccc2c1C(=O)c1c(O)c3c(c(O)c1C2=O)C[C@@](O)(C(=O)NC1CNC1)C[C@@H]3O[C@H]1C[C@H]2[C@H](O[C@@H]3[C@@H](OC)OCCN32)[C@H](C)O1] | 682 |
| 28 | 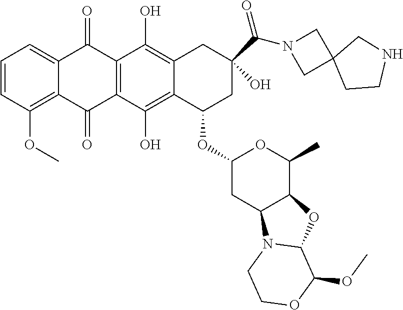 [SMILES: COc1cccc2c1C(=O)c1c(O)c3c(c(O)c1C2=O)C[C@@](O)(C(=O)N1CC2(CCNC2)C1)C[C@@H]3O[C@H]1C[C@H]2[C@H](O[C@@H]3[C@@H](OC)OCCN32)[C@H](C)O1] | 722 |
| 29 | 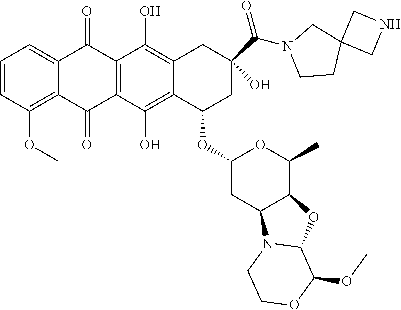 [SMILES: COc1cccc2c1C(=O)c1c(O)c3c(c(O)c1C2=O)C[C@@](O)(C(=O)N1CCC2(CNC2)C1)C[C@@H]3O[C@H]1C[C@H]2[C@H](O[C@@H]3[C@@H](OC)OCCN32)[C@H](C)O1] | 722 |
| 30 | 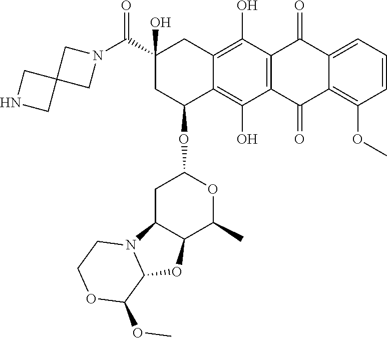 [SMILES: COc1cccc2c1C(=O)c1c(O)c3c(c(O)c1C2=O)C[C@@](O)(C(=O)N1CC2(CNC2)C1)C[C@@H]3O[C@H]1C[C@H]2[C@H](O[C@@H]3[C@@H](OC)OCCN32)[C@H](C)O1] | 708 |
| 31 | 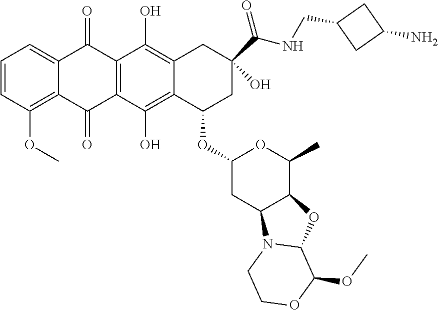 [SMILES: COc1cccc2c1C(=O)c1c(O)c3c(c(O)c1C2=O)C[C@@](O)(C(=O)NC[C@H]1C[C@@H](N)C1)C[C@@H]3O[C@H]1C[C@H]2[C@H](O[C@@H]3[C@@H](OC)OCCN32)[C@H](C)O1] | 710 |
| 32 | 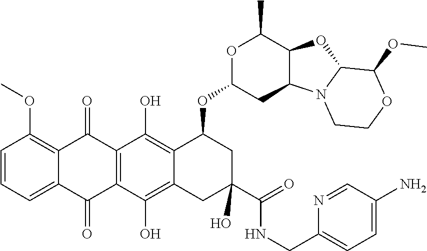 [SMILES: COc1cccc2c1C(=O)c1c(O)c3c(c(O)c1C2=O)C[C@@](O)(C(=O)NCc1ccc(N)cn1)C[C@@H]3O[C@H]1C[C@H]2[C@H](O[C@@H]3[C@@H](OC)OCCN32)[C@H](C)O1] | 733 |
| 33 | 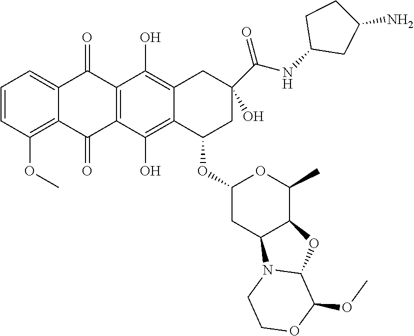 [SMILES: COc1cccc2c1C(=O)c1c(O)c3c(c(O)c1C2=O)C[C@@](O)(C(=O)N[C@@H]1CC[C@H](N)C1)C[C@@H]3O[C@H]1C[C@H]2[C@H](O[C@@H]3[C@@H](OC)OCCN32)[C@H](C)O1] | 710 |
| 34 | 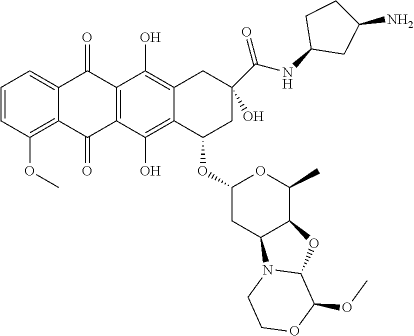 [SMILES: COc1cccc2c1C(=O)c1c(O)c3c(c(O)c1C2=O)C[C@@](O)(C(=O)N[C@H]1CC[C@@H](N)C1)C[C@@H]3O[C@H]1C[C@H]2[C@H](O[C@@H]3[C@@H](OC)OCCN32)[C@H](C)O1] | 710 |
| 35 | 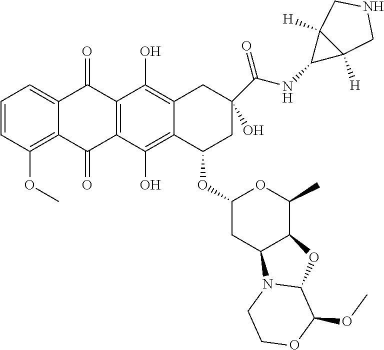 [SMILES: COc1cccc2c1C(=O)c1c(O)c3c(c(O)c1C2=O)C[C@@](O)(C(=O)N[C@H]1[C@@H]2CNC[C@@H]21)C[C@@H]3O[C@H]1C[C@H]2[C@H](O[C@@H]3[C@@H](OC)OCCN32)[C@H](C)O1] | 708 |
| 36 | 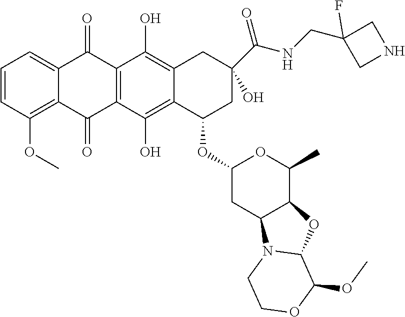 [SMILES: COc1cccc2c1C(=O)c1c(O)c3c(c(O)c1C2=O)C[C@@](O)(C(=O)NCC1(F)CNC1)C[C@@H]3O[C@H]1C[C@H]2[C@H](O[C@@H]3[C@@H](OC)OCCN32)[C@H](C)O1] | 714 |
| 37 | 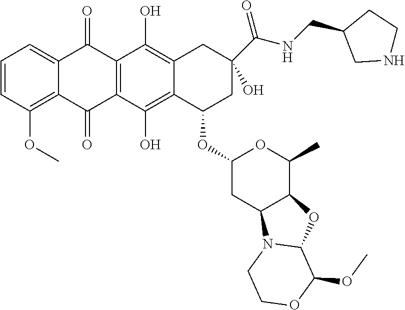 [SMILES: COc1cccc2c1C(=O)c1c(O)c3c(c(O)c1C2=O)C[C@@](O)(C(=O)NC[C@H]1CCNC1)C[C@@H]3O[C@H]1C[C@H]2[C@H](O[C@@H]3[C@@H](OC)OCCN32)[C@H](C)O1] | 710 |
| 38 | 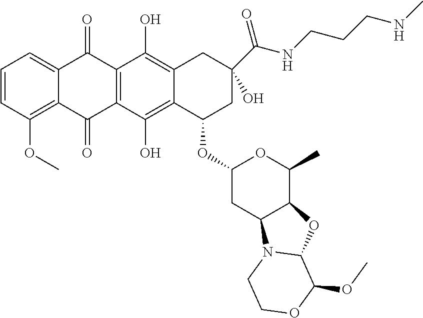 [SMILES: CNCCCNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O] | 698 |
| 39 | 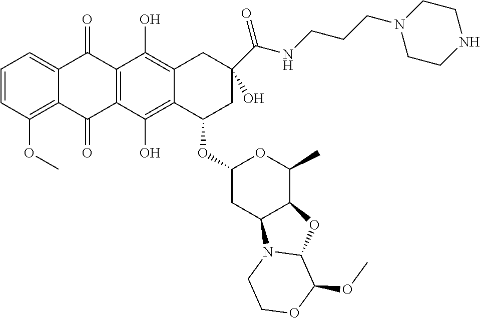 [SMILES: COc1cccc2c1C(=O)c1c(O)c3c(c(O)c1C2=O)C[C@@](O)(C(=O)NCCCN1CCNCC1)C[C@@H]3O[C@H]1C[C@H]2[C@H](O[C@@H]3[C@@H](OC)OCCN32)[C@H](C)O1] | 753 |
| 40 | 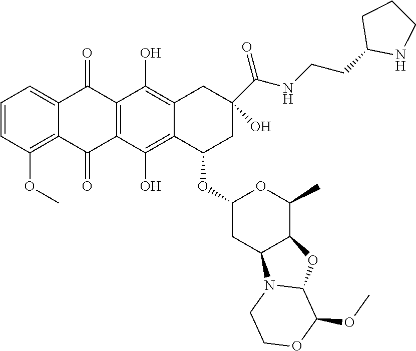 [SMILES: COc1cccc2c1C(=O)c1c(O)c3c(c(O)c1C2=O)C[C@@](O)(C(=O)NCC[C@@H]1CCCN1)C[C@@H]3O[C@H]1C[C@H]2[C@H](O[C@@H]3[C@@H](OC)OCCN32)[C@H](C)O1] | 724 |
| 41 | 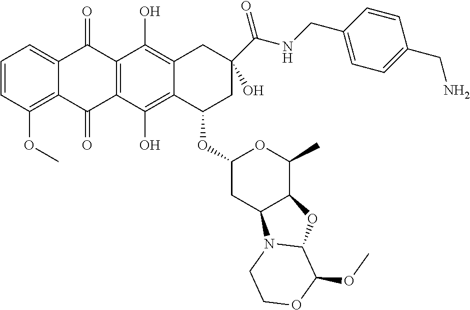 [SMILES: COc1cccc2c1C(=O)c1c(O)c3c(c(O)c1C2=O)C[C@@](O)(C(=O)NCc1ccc(CN)cc1)C[C@@H]3O[C@H]1C[C@H]2[C@H](O[C@@H]3[C@@H](OC)OCCN32)[C@H](C)O1] | 746 |
| 42 | 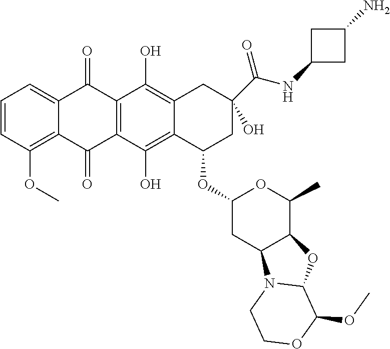 [SMILES: COc1cccc2c1C(=O)c1c(O)c3c(c(O)c1C2=O)C[C@@](O)(C(=O)N[C@H]1C[C@H](N)C1)C[C@@H]3O[C@H]1C[C@H]2[C@H](O[C@@H]3[C@@H](OC)OCCN32)[C@H](C)O1] | 696 |
| 43 | 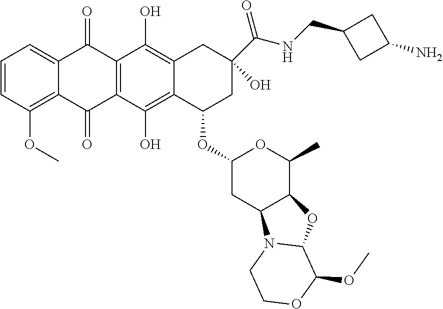 [SMILES: COc1cccc2c1C(=O)c1c(O)c3c(c(O)c1C2=O)C[C@@](O)(C(=O)NC[C@H]1C[C@H](N)C1)C[C@@H]3O[C@H]1C[C@H]2[C@H](O[C@@H]3[C@@H](OC)OCCN32)[C@H](C)O1] | 710 |
| 44 | 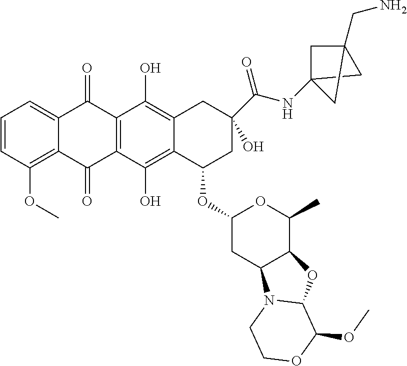 [SMILES: COc1cccc2c1C(=O)c1c(O)c3c(c(O)c1C2=O)C[C@@](O)(C(=O)NC12CC(CN)(C1)C2)C[C@@H]3O[C@H]1C[C@H]2[C@H](O[C@@H]3[C@@H](OC)OCCN32)[C@H](C)O1] | 722 |
| 45 | 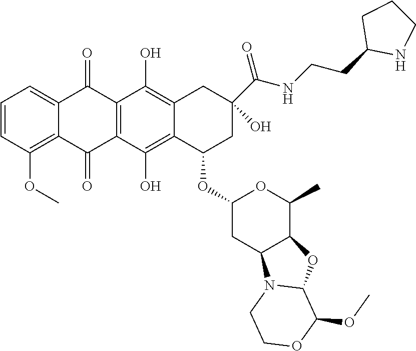 [SMILES: COc1cccc2c1C(=O)c1c(O)c3c(c(O)c1C2=O)C[C@@](O)(C(=O)NCC[C@H]1CCCN1)C[C@@H]3O[C@H]1C[C@H]2[C@H](O[C@@H]3[C@@H](OC)OCCN32)[C@H](C)O1] | 724 |
| 46 | 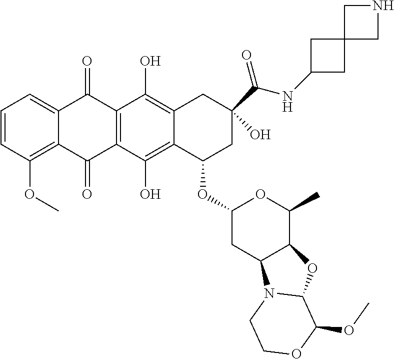 [SMILES: COc1cccc2c1C(=O)c1c(O)c3c(c(O)c1C2=O)C[C@@](O)(C(=O)NC1CC2(CNC2)C1)C[C@@H]3O[C@H]1C[C@H]2[C@H](O[C@@H]3[C@@H](OC)OCCN32)[C@H](C)O1] | 722 |
| 47 | 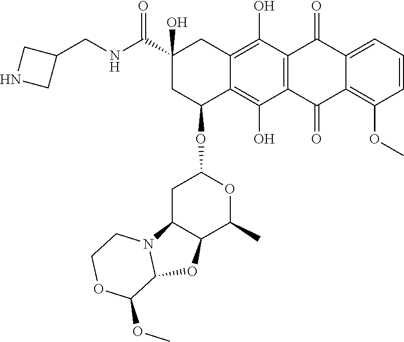 [SMILES: COc1cccc2c1C(=O)c1c(O)c3c(c(O)c1C2=O)C[C@@](O)(C(=O)NCC1CNC1)C[C@@H]3O[C@H]1C[C@H]2[C@H](O[C@@H]3[C@@H](OC)OCCN32)[C@H](C)O1] | 696 |
| 48 | 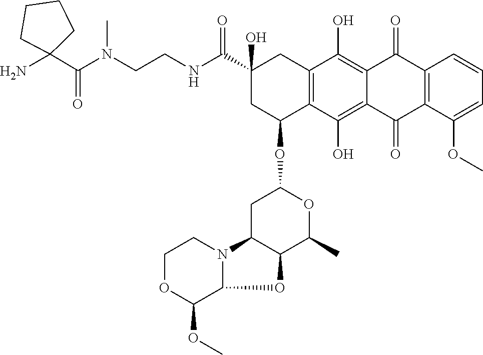 [SMILES: COc1cccc2c1C(=O)c1c(O)c3c(c(O)c1C2=O)C[C@@](O)(C(=O)NCCN(C)C(=O)C1(N)CCCC1)C[C@@H]3O[C@H]1C[C@H]2[C@H](O[C@@H]3[C@@H](OC)OCCN32)[C@H](C)O1] | 795 |
| 49 | 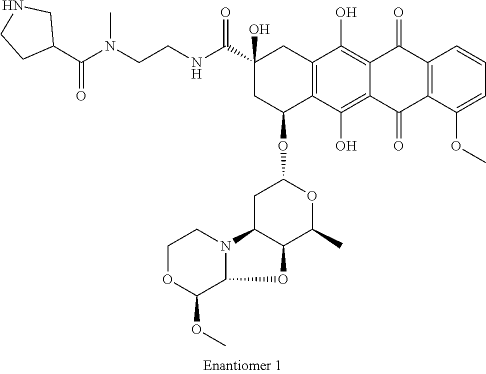 [SMILES: COc1cccc2c1C(=O)c1c(O)c3c(c(O)c1C2=O)C[C@@](O)(C(=O)NCCN(C)C(=O)C1CCNC1)C[C@@H]3O[C@H]1C[C@H]2[C@H](O[C@@H]3[C@@H](OC)OCCN32)[C@H](C)O1] | 781 |
| 50 | 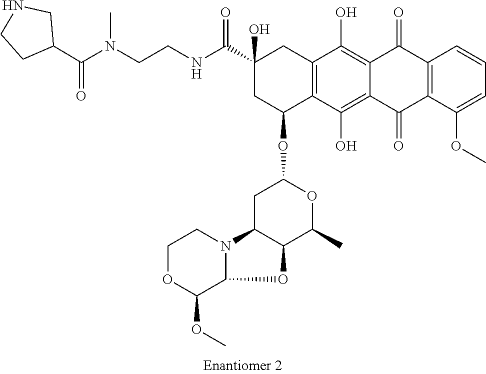 [SMILES: COc1cccc2c1C(=O)c1c(O)c3c(c(O)c1C2=O)C[C@@](O)(C(=O)NCCN(C)C(=O)C1CCNC1)C[C@@H]3O[C@H]1C[C@H]2[C@H](O[C@@H]3[C@@H](OC)OCCN32)[C@H](C)O1] | 781 |
| 51 | 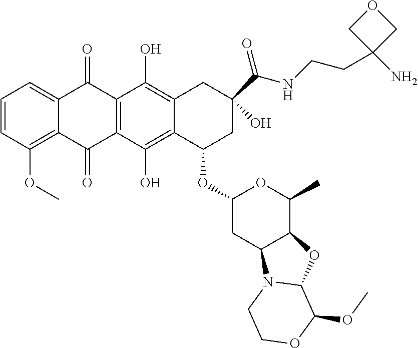 [SMILES: COc1cccc2c1C(=O)c1c(O)c3c(c(O)c1C2=O)C[C@@](O)(C(=O)NCCC1(N)COC1)C[C@@H]3O[C@H]1C[C@H]2[C@H](O[C@@H]3[C@@H](OC)OCCN32)[C@H](C)O1] | 726 |
| 52 | 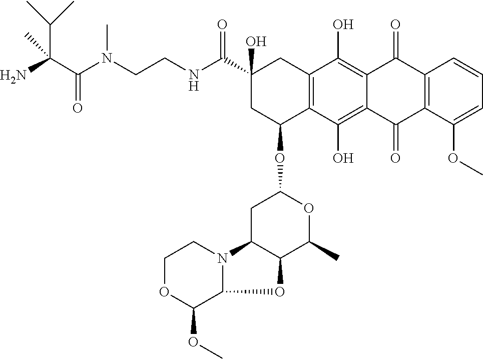 [SMILES: COc1cccc2c1C(=O)c1c(O)c3c(c(O)c1C2=O)C[C@@](O)(C(=O)NCCN(C)C(=O)[C@@](C)(N)C(C)C)C[C@@H]3O[C@H]1C[C@H]2[C@H](O[C@@H]3[C@@H](OC)OCCN32)[C@H](C)O1] | 797 |
| 53 | 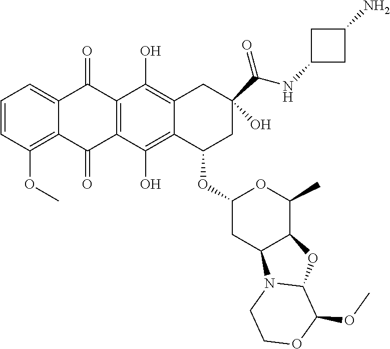 [SMILES: COc1cccc2c1C(=O)c1c(O)c3c(c(O)c1C2=O)C[C@@](O)(C(=O)N[C@H]1C[C@@H](N)C1)C[C@@H]3O[C@H]1C[C@H]2[C@H](O[C@@H]3[C@@H](OC)OCCN32)[C@H](C)O1] | 696 |
| 54 | 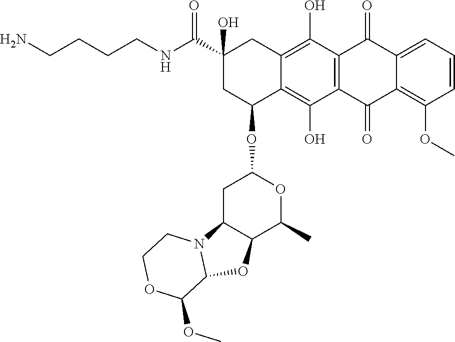 [SMILES: COc1cccc2c1C(=O)c1c(O)c3c(c(O)c1C2=O)C[C@@](O)(C(=O)NCCCCN)C[C@@H]3O[C@H]1C[C@H]2[C@H](O[C@@H]3[C@@H](OC)OCCN32)[C@H](C)O1] | 698 |
| 55 | 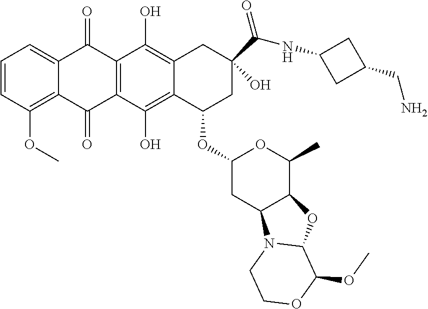 [SMILES: COc1cccc2c1C(=O)c1c(O)c3c(c(O)c1C2=O)C[C@@](O)(C(=O)N[C@H]1C[C@@H](CN)C1)C[C@@H]3O[C@H]1C[C@H]2[C@H](O[C@@H]3[C@@H](OC)OCCN32)[C@H](C)O1] | 710 |
| 56 | 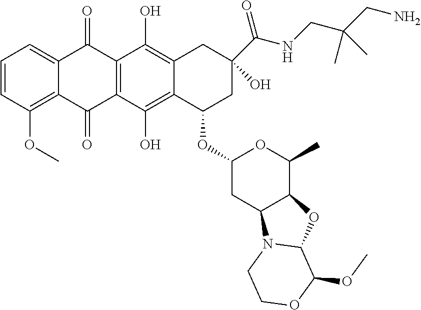 [SMILES: COc1cccc2c1C(=O)c1c(O)c3c(c(O)c1C2=O)C[C@@](O)(C(=O)NCC(C)(C)CN)C[C@@H]3O[C@H]1C[C@H]2[C@H](O[C@@H]3[C@@H](OC)OCCN32)[C@H](C)O1] | 712 |
| 57 | 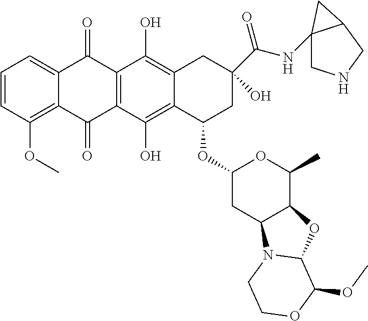 [SMILES: COc1cccc2c1C(=O)c1c(O)c3c(c(O)c1C2=O)C[C@@](O)(C(=O)NC12CNCC1C2)C[C@@H]3O[C@H]1C[C@H]2[C@H](O[C@@H]3[C@@H](OC)OCCN32)[C@H](C)O1] | 708 |
| 58 | 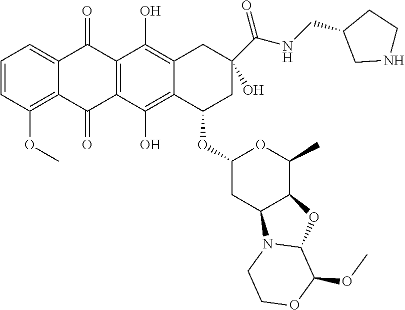 [SMILES: COc1cccc2c1C(=O)c1c(O)c3c(c(O)c1C2=O)C[C@@](O)(C(=O)NC[C@@H]1CCNC1)C[C@@H]3O[C@H]1C[C@H]2[C@H](O[C@@H]3[C@@H](OC)OCCN32)[C@H](C)O1] | 710 |
| 59 | 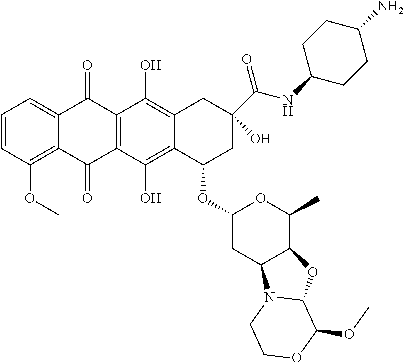 [SMILES: COc1cccc2c1C(=O)c1c(O)c3c(c(O)c1C2=O)C[C@@](O)(C(=O)N[C@H]1CC[C@H](N)CC1)C[C@@H]3O[C@H]1C[C@H]2[C@H](O[C@@H]3[C@@H](OC)OCCN32)[C@H](C)O1] | 724 |
| 60 | 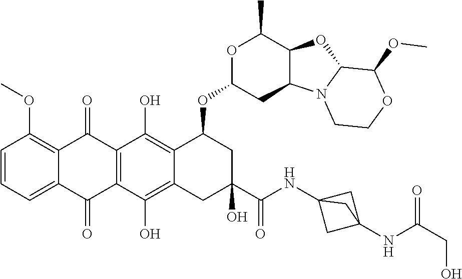 [SMILES: COc1cccc2c1C(=O)c1c(O)c3c(c(O)c1C2=O)C[C@@](O)(C(=O)NC12CC(NC(=O)CO)(C1)C2)C[C@@H]3O[C@H]1C[C@H]2[C@H](O[C@@H]3[C@@H](OC)OCCN32)[C@H](C)O1] | 766 |

### Example 14

### Preparation of Compounds 61-90

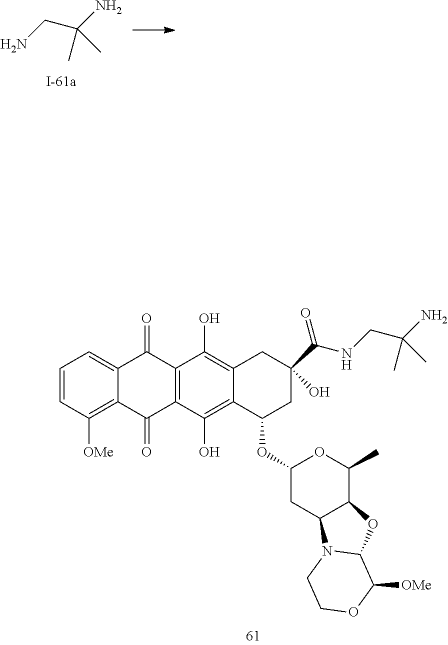

To a solution of (9H-fluoren-9-yl)methyl (3-(aminomethyl)phenyl)carbamate (150 mg. 0.239 mmol) in DMF (1 mL) was added HATU (109 mg, 0.287 mmol). The resulting solution was allowed to stir for 10 minutes at room temperature, then a solution of 2-methylpropane-1,2-diamine (I-61a. 21.1 mg, 0.239 mmol) in DMF (0.2 mL) was added, and the resulting reaction was allowed to stir for 10 minutes at room temperature. DIEA (93 mg. 0.72 mmol) was added, and the resulting reaction was allowed to stir at 25° C. for 20 minutes. The reaction mixture was then concentrated in vacuo, and the residue obtained was purified using preparative HPLC (Column: C18-1, 150×30 mm×5 um, eluting with 45% to 75% ACN:water (with 7 mM HCOONH₄ modifier)) to provide (2S,4S)—N-(2-amino-2-methylpropyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (61, 29.8 mg, 0.041 mmol) as a solid. LCMS (ESI) m/z: 698.3 [M+H]⁺. ¹H NMR (400 MHz, CDCl₃) δ=13.73 (s, 1H), 8.44 (s, 2H), 8.06 (br s, 1H), 7.89 (d, J=7.6 Hz, 1H), 7.68 (t, J=8.1 Hz, 1H), 7.23 (br d, J=8.5 Hz, 1H), 5.47 (br t, J=5.1 Hz, 1H), 5.19 (br s, 1H), 4.71 (d, J=1.4 Hz, 1H), 4.48 (d, J=1.5 Hz, 1H), 4.09 (br d, J=6.6 Hz, 2H), 3.98-3.87 (m, 4H), 3.62-3.53 (m, 2H), 3.48-3.39 (m, 5H), 3.21-3.02 (m, 2H), 2.86-2.67 (m, 2H), 2.47-2.24 (m, 2H), 2.04-1.90 (m, 1H). 1.90-1.81 (m, 1H), 1.47-1.39 (m, 8H), 1.35 (br d, J=6.4 Hz, 3H).

The following illustrative compounds of the present disclosure were made using the methods described in Example 14, and substituting the appropriate reactants and/or reagents:

| Com- | | MS |
|---|---|---|
| pound | Structure | [M + H] |
| 62 | 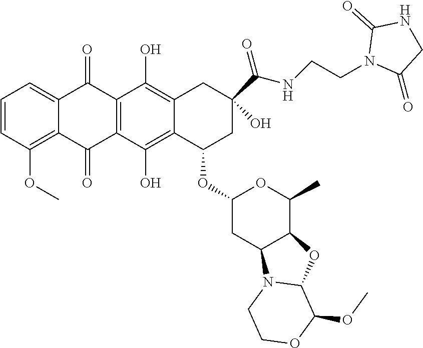 [SMILES: COc1cccc2c1C(=O)c1c(O)c3c(c(O)c1C2=O)C[C@@](O)(C(=O)NCCN1C(=O)CNC1=O)C[C@@H]3O[C@H]1C[C@H]2[C@H](O[C@@H]3[C@@H](OC)OCCN32)[C@H](C)O1] | 753 |
| 63 | 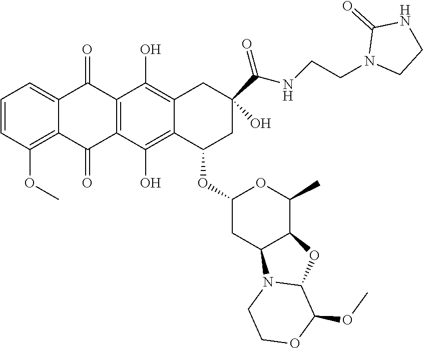 [SMILES: COc1cccc2c1C(=O)c1c(O)c3c(c(O)c1C2=O)C[C@@](O)(C(=O)NCCN1CCNC1=O)C[C@@H]3O[C@H]1C[C@H]2[C@H](O[C@@H]3[C@@H](OC)OCCN32)[C@H](C)O1] | 739 |
| 64 | 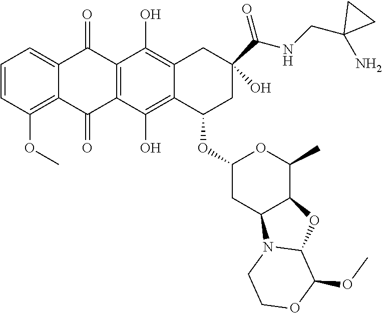 [SMILES: COc1cccc2c1C(=O)c1c(O)c3c(c(O)c1C2=O)C[C@@](O)(C(=O)NCC1(N)CC1)C[C@@H]3O[C@H]1C[C@H]2[C@H](O[C@@H]3[C@@H](OC)OCCN32)[C@H](C)O1] | 696 |
| 65 | 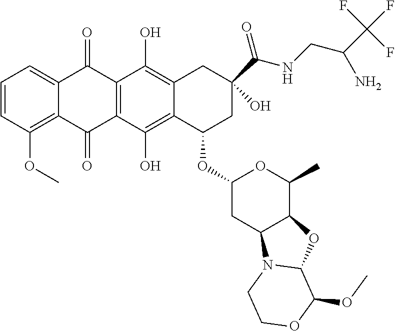 [SMILES: COc1cccc2c1C(=O)c1c(O)c3c(c(O)c1C2=O)C[C@@](O)(C(=O)NCC(N)C(F)(F)F)C[C@@H]3O[C@H]1C[C@H]2[C@H](O[C@@H]3[C@@H](OC)OCCN32)[C@H](C)O1] | 738 |
| 66 | 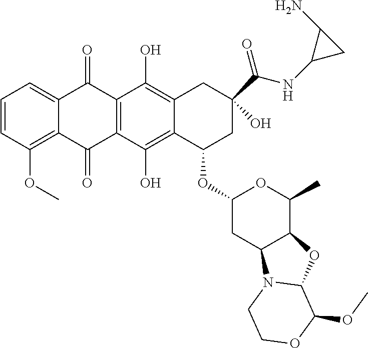 [SMILES: COc1cccc2c1C(=O)c1c(O)c3c(c(O)c1C2=O)C[C@@](O)(C(=O)NC1CC1N)C[C@@H]3O[C@H]1C[C@H]2[C@H](O[C@@H]3[C@@H](OC)OCCN32)[C@H](C)O1] | 682 |
| | Enantiomer 1 | |
| 67 | 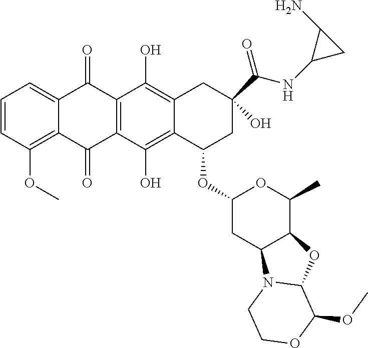 [SMILES: COc1cccc2c1C(=O)c1c(O)c3c(c(O)c1C2=O)C[C@@](O)(C(=O)NC1CC1N)C[C@@H]3O[C@H]1C[C@H]2[C@H](O[C@@H]3[C@@H](OC)OCCN32)[C@H](C)O1] | 682 |
| | Enantiomer 2 | |
| 68 | 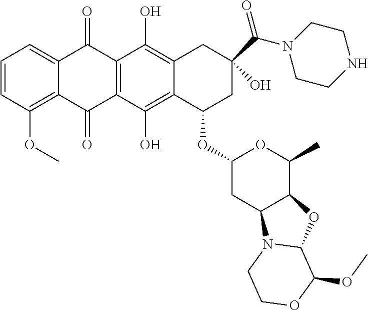 [SMILES: COc1cccc2c1C(=O)c1c(O)c3c(c(O)c1C2=O)C[C@@](O)(C(=O)N1CCNCC1)C[C@@H]3O[C@H]1C[C@H]2[C@H](O[C@@H]3[C@@H](OC)OCCN32)[C@H](C)O1] | 696 |
| 69 | 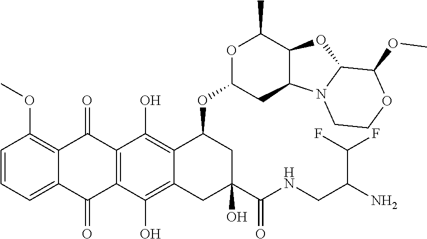 [SMILES: COc1cccc2c1C(=O)c1c(O)c3c(c(O)c1C2=O)C[C@@](O)(C(=O)NCC(N)C(F)F)C[C@@H]3O[C@H]1C[C@H]2[C@H](O[C@@H]3[C@@H](OC)OCCN32)[C@H](C)O1] | 720 |
| 70 | 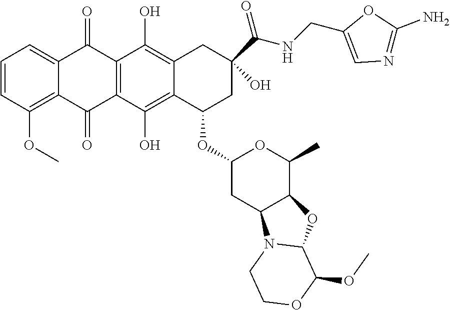 [SMILES: COc1cccc2c1C(=O)c1c(O)c3c(c(O)c1C2=O)C[C@@](O)(C(=O)NCc1cnc(N)o1)C[C@@H]3O[C@H]1C[C@H]2[C@H](O[C@@H]3[C@@H](OC)OCCN32)[C@H](C)O1] | 723 |
| 71 | 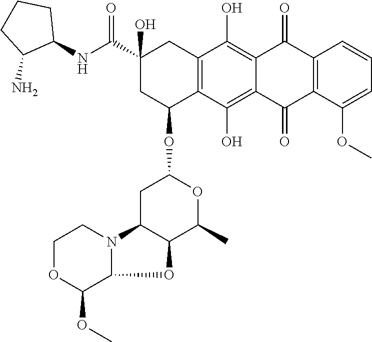 [SMILES: COc1cccc2c1C(=O)c1c(O)c3c(c(O)c1C2=O)C[C@@](O)(C(=O)N[C@@H]1CCC[C@H]1N)C[C@@H]3O[C@H]1C[C@H]2[C@H](O[C@@H]3[C@@H](OC)OCCN32)[C@H](C)O1] | 710 |
| 72 | 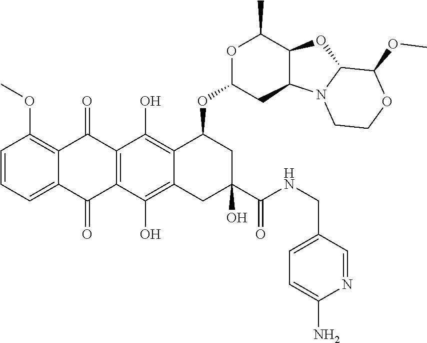 [SMILES: COc1cccc2c1C(=O)c1c(O)c3c(c(O)c1C2=O)C[C@@](O)(C(=O)NCc1ccc(N)nc1)C[C@@H]3O[C@H]1C[C@H]2[C@H](O[C@@H]3[C@@H](OC)OCCN32)[C@H](C)O1] | 733 |
| 73 | 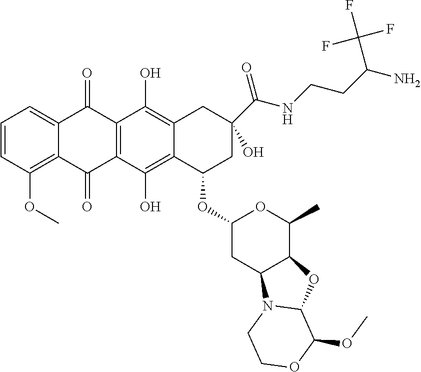 [SMILES: COc1cccc2c1C(=O)c1c(O)c3c(c(O)c1C2=O)C[C@@](O)(C(=O)NCCC(N)C(F)(F)F)C[C@@H]3O[C@H]1C[C@H]2[C@H](O[C@@H]3[C@@H](OC)OCCN32)[C@H](C)O1] | 752 |
| 74 | 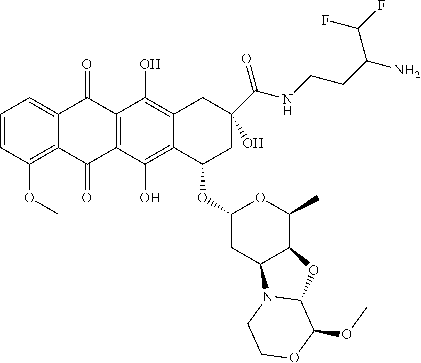 [SMILES: COc1cccc2c1C(=O)c1c(O)c3c(c(O)c1C2=O)C[C@@](O)(C(=O)NCCC(N)C(F)F)C[C@@H]3O[C@H]1C[C@H]2[C@H](O[C@@H]3[C@@H](OC)OCCN32)[C@H](C)O1] | 734 |
| 75 | 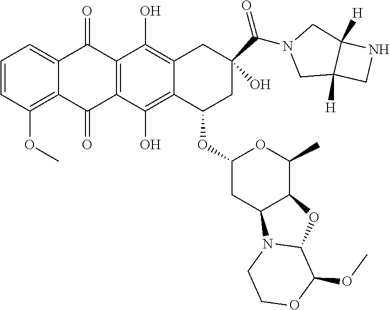 [SMILES: COc1cccc2c1C(=O)c1c(O)c3c(c(O)c1C2=O)C[C@@](O)(C(=O)N1C[C@H]2CN[C@H]2C1)C[C@@H]3O[C@H]1C[C@H]2[C@H](O[C@@H]3[C@@H](OC)OCCN32)[C@H](C)O1] | 708 |
| 76 | 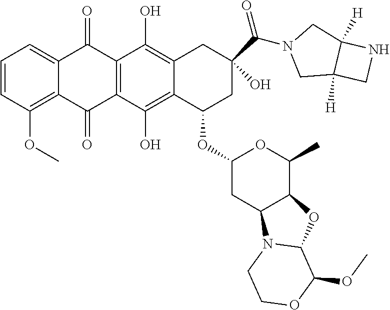 [SMILES: COc1cccc2c1C(=O)c1c(O)c3c(c(O)c1C2=O)C[C@@](O)(C(=O)N1C[C@@H]2CN[C@@H]2C1)C[C@@H]3O[C@H]1C[C@H]2[C@H](O[C@@H]3[C@@H](OC)OCCN32)[C@H](C)O1] | 708 |
| 77 | 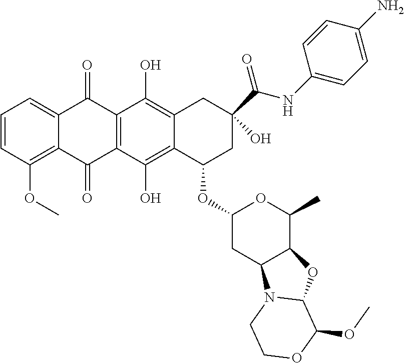 [SMILES: COc1cccc2c1C(=O)c1c(O)c3c(c(O)c1C2=O)C[C@@](O)(C(=O)Nc1ccc(N)cc1)C[C@@H]3O[C@H]1C[C@H]2[C@H](O[C@@H]3[C@@H](OC)OCCN32)[C@H](C)O1] | 718 |
| 78 | 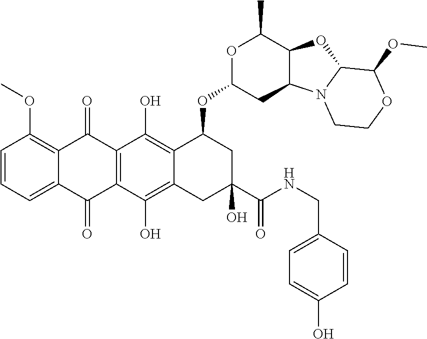 [SMILES: COc1cccc2c1C(=O)c1c(O)c3c(c(O)c1C2=O)C[C@@](O)(C(=O)NCc1ccc(O)cc1)C[C@@H]3O[C@H]1C[C@H]2[C@H](O[C@@H]3[C@@H](OC)OCCN32)[C@H](C)O1] | 733 |
| 79 | 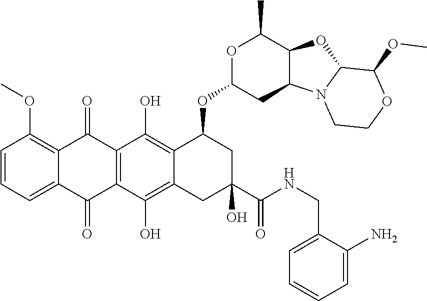 [SMILES: COc1cccc2c1C(=O)c1c(O)c3c(c(O)c1C2=O)C[C@@](O)(C(=O)NCc1ccccc1N)C[C@@H]3O[C@H]1C[C@H]2[C@H](O[C@@H]3[C@@H](OC)OCCN32)[C@H](C)O1] | 732 |
| 80 | 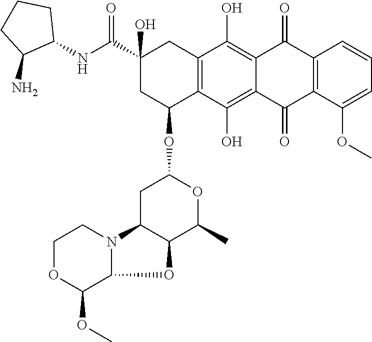 [SMILES: COc1cccc2c1C(=O)c1c(O)c3c(c(O)c1C2=O)C[C@@](O)(C(=O)N[C@H]1CCC[C@@H]1N)C[C@@H]3O[C@H]1C[C@H]2[C@H](O[C@@H]3[C@@H](OC)OCCN32)[C@H](C)O1] | 710 |
| 81 | 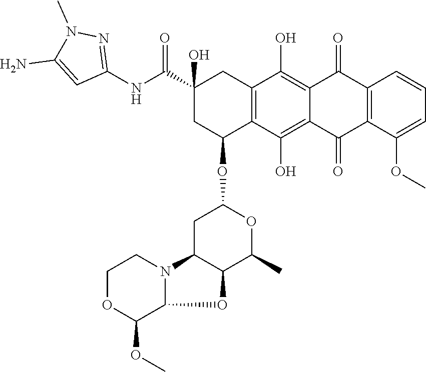 [SMILES: COc1cccc2c1C(=O)c1c(O)c3c(c(O)c1C2=O)C[C@@](O)(C(=O)Nc1cc(N)n(C)n1)C[C@@H]3O[C@H]1C[C@H]2[C@H](O[C@@H]3[C@@H](OC)OCCN32)[C@H](C)O1] | 722 |
| 82 | 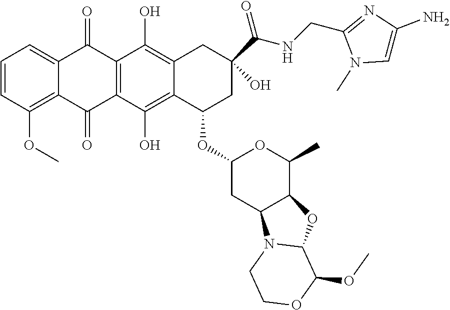 [SMILES: COc1cccc2c1C(=O)c1c(O)c3c(c(O)c1C2=O)C[C@@](O)(C(=O)NCc1nc(N)cn1C)C[C@@H]3O[C@H]1C[C@H]2[C@H](O[C@@H]3[C@@H](OC)OCCN32)[C@H](C)O1] | 736 |
| 83 | 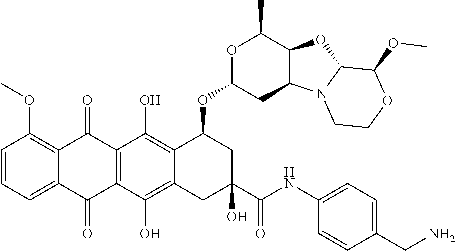 [SMILES: COc1cccc2c1C(=O)c1c(O)c3c(c(O)c1C2=O)C[C@@](O)(C(=O)Nc1ccc(CN)cc1)C[C@@H]3O[C@H]1C[C@H]2[C@H](O[C@@H]3[C@@H](OC)OCCN32)[C@H](C)O1] | 732 |
| 84 | 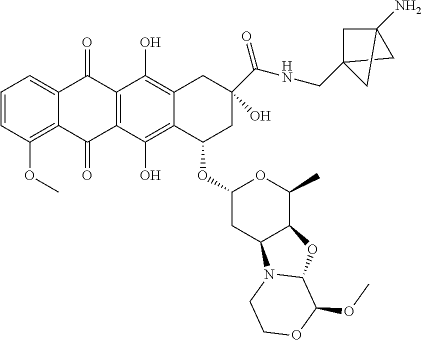 [SMILES: COc1cccc2c1C(=O)c1c(O)c3c(c(O)c1C2=O)C[C@@](O)(C(=O)NCC12CC(N)(C1)C2)C[C@@H]3O[C@H]1C[C@H]2[C@H](O[C@@H]3[C@@H](OC)OCCN32)[C@H](C)O1] | 722 |
| 85 | 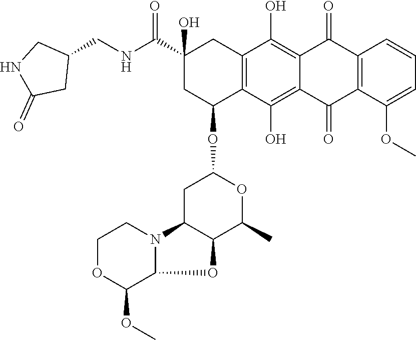 [SMILES: COc1cccc2c1C(=O)c1c(O)c3c(c(O)c1C2=O)C[C@@](O)(C(=O)NC[C@H]1CNC(=O)C1)C[C@@H]3O[C@H]1C[C@H]2[C@H](O[C@@H]3[C@@H](OC)OCCN32)[C@H](C)O1] | 724 |
| 86 | 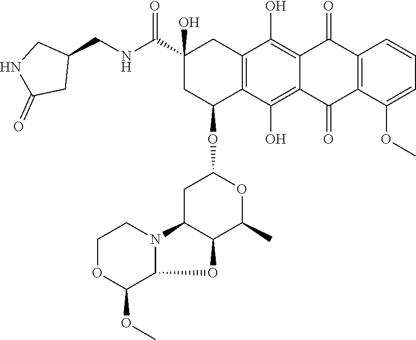 [SMILES: COc1cccc2c1C(=O)c1c(O)c3c(c(O)c1C2=O)C[C@@](O)(C(=O)NC[C@@H]1CNC(=O)C1)C[C@@H]3O[C@H]1C[C@H]2[C@H](O[C@@H]3[C@@H](OC)OCCN32)[C@H](C)O1] | 724 |
| 87 | 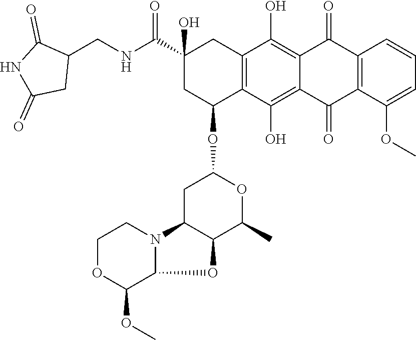 [SMILES: COc1cccc2c1C(=O)c1c(O)c3c(c(O)c1C2=O)C[C@@](O)(C(=O)NCC1CC(=O)NC1=O)C[C@@H]3O[C@H]1C[C@H]2[C@H](O[C@@H]3[C@@H](OC)OCCN32)[C@H](C)O1] | 738 |
| 88 | 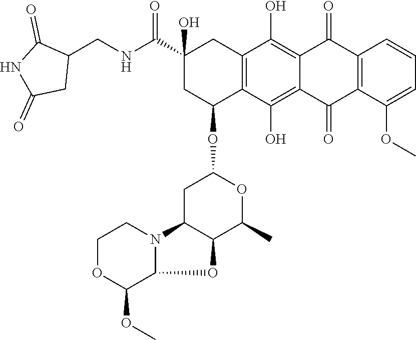 [SMILES: COc1cccc2c1C(=O)c1c(O)c3c(c(O)c1C2=O)C[C@@](O)(C(=O)NCC1CC(=O)NC1=O)C[C@@H]3O[C@H]1C[C@H]2[C@H](O[C@@H]3[C@@H](OC)OCCN32)[C@H](C)O1] | 738 |
| 89 | 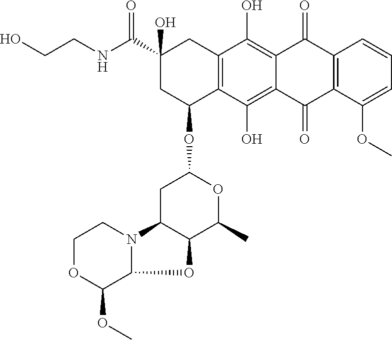 [SMILES: COc1cccc2c1C(=O)c1c(O)c3c(c(O)c1C2=O)C[C@@](O)(C(=O)NCCO)C[C@@H]3O[C@H]1C[C@H]2[C@H](O[C@@H]3[C@@H](OC)OCCN32)[C@H](C)O1] | 671 |
| 90 | 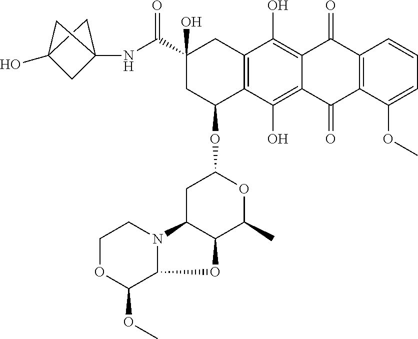 [SMILES: COc1cccc2c1C(=O)c1c(O)c3c(c(O)c1C2=O)C[C@@](O)(C(=O)NC12CC(O)(C1)C2)C[C@@H]3O[C@H]1C[C@H]2[C@H](O[C@@H]3[C@@H](OC)OCCN32)[C@H](C)O1] | 709 |

### Example 15

### CellTiter-Glo® 2.0 Cytotoxicity Assay

### Step 1: Seeding of Plates

On day zero, Jeko-1 cells were quickly thawed in a cryo-vial by incubating them in a 37° C. water bath for <1 min until there is just a small bit of ice left in the vial. The vial was promptly removed and wiped down with 70% ethanol. The cells were transferred from the vial to a sterile centrifuge tube containing 8 mL of pre-warmed cell culture medium. The vial was flushed with an additional 1 mL of medium to ensure complete transfer of cells to the centrifuge tube. The cells were then centrifuged at 150×g for 5 minutes. The supernatant was aspirated, and the cell pellet was resuspended in 10-20 mL cell culture medium. Cells were counted using Vi-cell and prepared 6.6×10⁴ cells/ml, 3000 cells/45 uL per well. Then added 45 uL/well of cells into Corning® 384-well Low Flange White Flat Bottom Polystyrene TC-treated Microplates (Corning, Cat #3570) using Standard Cassette Combi (if needed, dispense 1 dummy plate at 20 uL to help normalize Combi, using medium speed). The plates were spun down at 150×g for 30 seconds.

### Step 2: Addition of Illustrative Compounds to Seeded Plates

On day 1, the compound plates and reference compound stock were taken out and allowed to thaw at room temperature. The tubes were centrifuged at 2000×g for 30 seconds. The 10× Intermediate assay plates (Greiner plate, Cat #781280) were prepared using an Echo liquid handler. Proper buffer (HBSS (Gibco, Cat #14025-092)+10 mM HEPES (Gibco, Cat #15630-080)+0.1% BSA (Sigma, Cat #A9576)) were used to make serial dilutions. Media (no cells) was used for Max_E. Compound (5 uL of 10×) was transferred from intermediate plate to assay plate using a Bravo liquid handler using a very slow speed so the cell monolayer wasn't disturbed. The plates were spun down at 150×g for 30 seconds.

### Step 3: Perform CellTiter-Glo 2.0 Assay (Proinega, Cat #G9242)

On day 4 or 5, the CellTiter-Glo® 2.0 Reagent (from CellTiter-Glo kit that was stored at −70° C.) was thawed at 4° C. overnight, taking care not to expose the reagent to temperatures above 25° C.). The kit was equilibrated to room temperature for approximately 30 minutes. CellTiter-Glo® 2.0 Reagent (20l) was added to 50 μl of medium containing cells using Standard Cassette Combi. The contents were mixed for 2-3 minutes on an orbital shaker to induce cell lysis. The plates were spun down at 150×g for 30 seconds. The plates were allowed to incubate at room temperature for 5 minutes to stabilize the luminescent signal. The luminescence was recorded to calculate an EC₅₀ value, using an integration time of 0.25-1 second per well as a guideline.

Illustrative compounds of the present disclosure were tested in the above assay, and results are provided in the Table below:

| | BxPC3 |
|---|---|
| Compound | EC₅₀ (nM) |
| 1 | 0.08 |
| 2 | 22 |
| 3 | 13 |
| 4 | 13 |
| 5 | 2.8 |
| 6 | 8.6 |
| 7 | 13 |
| 8 | 48 |
| 9 | 17 |
| 10 | 124 |
| 11 | 821.5 |
| 12 | 7.7 |
| 13 | 5.4 |
| 14 | 11 |
| 15 | 64 |
| 16 | 24.0 |
| 17 | 10.7 |
| 18 | 8.5 |
| 19 | 4.0 |
| 20 | 8.5 |
| 23 | 0.06 |
| 24 | 8.3 |
| 25 | 10.5 |
| 26 | 2.7 |
| 27 | 39 |
| 28 | 478 |
| 29 | 90 |
| 30 | 14 |
| 31 | 2.6 |
| 32 | 0.07 |
| 33 | 9.2 |
| 34 | 19 |
| 35 | 0.97 |
| 36 | 0.42 |
| 37 | 8.0 |
| 38 | 9.1 |
| 39 | 3.8 |
| 40 | 4.3 |
| 41 | 0.42 |
| 42 | 12 |
| 43 | 2.2 |
| 44 | 4.8 |
| 45 | 0.6 |
| 46 | 18 |
| 47 | 9.4 |
| 48 | 0.31 |
| 49 | 31 |
| 50 | 20 |
| 51 | 0.18 |
| 52 | 1.9 |
| 53 | 7.7 |
| 54 | 1.4 |
| 55 | 10.2 |
| 56 | 1.4 |
| 57 | 1.6 |
| 58 | 3.9 |
| 59 | 6.3 |
| 60 | 0.55 |
| 61 | 8.7 |
| 62 | 0.19 |
| 63 | 0.14 |
| 64 | 0.49 |
| 65 | 0.16 |
| 68 | 8.1 |
| 69 | 0.16 |
| 70 | 0.095 |
| 71 | 8.4 |
| 72 | 0.08 |
| 73 | 0.20 |
| 74 | 0.20 |
| 75 | 2.9 |
| 76 | 15 |
| 77 | 0.11 |
| 78 | 0.056 |
| 79 | 0.15 |
| 80 | 14.1 |
| 81 | 0.40 |
| 82 | 0.26 |
| 83 | 11 |
| 84 | 0.61 |
| 85 | 0.40 |
| 86 | 0.39 |
| 87 | 0.12 |
| 88 | 0.27 |
| 89 | 0.37 |
| 90 | 0.23 |

### Uses of the PNU Anthracycline Derivatives

### Treatment or Prevention of Cellular Proliferation Disorders

The present disclosure also relates to methods of treating a cellular proliferative disorder, said methods comprising administering to a subject in need thereof a PNU Anthracycline Derivative.

The PNU Anthracycline Derivatives disclosed herein are potentially useful in treating diseases or disorders including, but not limited to, cellular proliferative disorders. Cellular proliferation disorders include, but are not limited to, cancers, benign papillomatosis, and gestational trophoblastic diseases. The terms “cancer”, “cancerous”, or “malignant” refer to or describe the physiological condition in mammals that is typically characterized by unregulated cell growth.

In specific embodiments, the cellular proliferative disorder is selected from cancer, benign papillomatosis, benign neoplastic diseases and gestational trophoblastic diseases. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In a particular embodiment, the cellular proliferative disorder being treated is cancer.

Accordingly, in one embodiment, provided herein are methods for treating cancer in a patient, the methods comprising administering to the patient an effective amount of a PNU Anthracycline Derivative. In a specific embodiment, the amount administered is effective to treat cancer in the patient. In another specific embodiment, the amount administered is effective to inhibit cancer cell replication or cancer cell metastasis in the patient.

In one embodiment, described herein are the use of the PNU Anthracycline Derivatives, or a pharmaceutically acceptable salt thereof, for the preparation of a medicament for the treatment of cancer.

In another embodiment, described herein are PNU Anthracycline Derivatives, for use in the treatment of cancer.

In one embodiment, the cancer is metastatic. In another embodiment, the cancer is relapsed. In another embodiment, the cancer is refractory. In yet another embodiment, the cancer is relapsed and refractory.

In one embodiment, the patient has previously received treatment for cancer. In another embodiment, the patient has not previously received treatment for cancer.

In one embodiment, the patient has previously received systemic treatment for cancer. In another embodiment, the patient has not previously received systemic treatment for cancer.

In other embodiments, the cancer is present in an adult patient; in additional embodiments, the cancer is present in a pediatric patient.

The compounds, compositions and methods provided herein are useful for the treatment of cancer. Cancers that may be treated using the compounds, compositions and methods disclosed herein include, but are not limited to: (1) Cardiac: sarcoma (angiosarcoma, fibrosarcoma, rhabdomyosarcoma, liposarcoma), myxoma, rhabdomyoma, fibroma, lipoma and teratoma; (2) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell; (3) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal: (4) Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); (5) Liver: hepatoma (hepatocellular carcinoma), cholangiocarcinoma, hepatoblastoma, angiosarcoma, hepatocellular adenoma, hemangioma; (6) Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; (7) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiforme, oligodendroglioma, schwannoma, retinoblastoma, congenital tumors), spinal cord neurofibroma, meningioma, glioma, sarcoma); (8) Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; (9) Hematologic: blood (myeloid leukemia [acute and chronic], acute lymphoblastic leukemia, chronic lymphocytic leukemia, chronic myelomonocytic (CMML), myelocellular proliferative disorders, multiple myeloma, myelodysplastic syndrome), Hodgkin's disease, non-Hodgkin's lymphoma [malignant lymphoma]; (10) Skin: malignant melanoma, basal cell carcinoma, squamous cell carcinoma, Karposi's sarcoma, moles dysplastic nevi, lipoma, angioma, dermatofibroma, keloids, psoriasis; and (11) Adrenal glands: neuroblastoma. Examples of cancer that may be treated using the compounds, compositions and methods described herein include thyroid cancer, anaplastic thyroid carcinoma, epidermal cancer, head and neck cancer (e.g., squamous cell cancer of the head and neck), sarcoma, tetracarcinoma, hepatoma and multiple myeloma.

The term “cancerous cell” as used herein, includes a cell afflicted by any, one of the above-identified conditions.

In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, metastatic microsatellite instability-high (MSI-H) cancer, mismatch repair deficient cancer, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary origin (i.e., cancers in which a metastasized cancer is found but the original cancer site is not known). In particular embodiments, the cancer is AIDS-related.

In one embodiment, the cancer is bladder cancer. In another embodiment, the cancer is breast cancer. In yet another embodiment, the cancer is NSCLC. In still another embodiment, the cancer is CRC. In another embodiment, the cancer is RCC. In another embodiment, the cancer is HCC. In one embodiment, the cancer is skin cancer. In another embodiment, the skin cancer is melanoma. In another embodiment, the cancer is ovarian cancer. In yet another embodiment, the cancer is pancreatic cancer. In another embodiment, the cancer is a primary or metastatic brain cancer. In still another embodiment, the cancer is CRC.

In one embodiment, provided herein is a method of treating unresectable or metastatic melanoma in a human patient. In some embodiments, the method comprises treating resected high-risk stage III melanoma.

In one embodiment, provided herein is a method of treating metastatic non-small cell lung cancer (NSCLC) in a human patient. In some embodiments, the NSCLC is non-squamous. In other embodiments, the NSCLC is squamous.

In some embodiments, the cancer exhibits high PD-L1 expression [(Tumor Proportion Score (TPS)≥50%)] and was not previously treated with platinum-containing chemotherapy. In alternative embodiments, the patient has a tumor with PD-L1 expression (TPS≥1%), and was previously treated with platinum-containing chemotherapy. In specific embodiments, the patient had disease progression on or after receiving platinum-containing chemotherapy.

In certain embodiments the PD-L1 TPS is determined by an FDA-approved test.

In certain embodiments of the method for treating NSCLC, the patient's tumor has no EGFR or ALK genomic aberrations.

In certain embodiments of the method for treating NSCLC, the patient's tumor has an EGFR or ALK genomic aberration and had disease progression on or after receiving treatment for the EGFR or ALK aberration(s) prior to receiving combination therapy described herein.

In one embodiment, provided herein is a method of treating recurrent or metastatic head and neck squamous cell cancer (HNSCC) in a human patient. In some embodiments, the patient was previously treated with platinum-containing chemotherapy. In certain embodiments, the patient had disease progression during or after platinum-containing chemotherapy.

In one embodiment, provided herein is a method of treating refractory classical Hodgkin lymphoma (cHL) in a human patient. In certain embodiments, the patient has relapsed after 1, 2, 3 or more lines of therapy for cHL. In specific embodiments, the patient is an adult patient. In alternative embodiments the patient is a pediatric patient.

In one embodiment, provided herein is a method of treating locally advanced or metastatic urothelial carcinoma in a human patient. In certain embodiments, the patient is not eligible for cisplatin-containing chemotherapy. In further embodiments, the patient has disease progression during or following platinum-containing chemotherapy or within 12 months of neoadjuvant or adjuvant treatment with platinum-containing chemotherapy. In specific embodiments, the patient's tumor expresses PD-L1 (CPS≥10).

In one embodiment, provided herein is a method of treating unresectable or metastatic, microsatellite instability-high (MSI-H) or mismatch repair deficient solid tumors in a human patient. In specific embodiments, the patient had disease progression following prior anti-cancer treatment.

In one embodiment, provided herein is a method of treating unresectable or metastatic, microsatellite instability-high (MSI-H) or mismatch repair deficient colorectal cancer in a human patient. In specific embodiments, the patient had disease progression following prior treatment with a fluoropyrimidine, oxaliplatin, and irinotecan.

In one embodiment, provided herein is a method of treating recurrent locally advanced or metastatic gastric cancer or recurrent locally advanced or metastatic gastroesophageal junction adenocarcinoma in a human patient. In specific embodiments, the patient's tumor expresses PD-L1 [Combined Positive Score (CPS)≥1]. In some embodiments, the patient has disease progression on or after two or more prior lines of therapy including fluoropyrimidine- and platinum-containing chemotherapy. In some embodiments, the patient has disease progression on or after two or more prior lines of therapy including HER2/neu-targeted therapy.

In one embodiment, provided herein is a method of treating non-Hodgkin lymphoma in a human patient. In certain embodiments, the non-Hodgkin lymphoma is primary mediastinal large B-cell lymphoma.

In one embodiment, provided herein is a method of treating breast cancer in a human patient. in specific embodiments, the breast cancer is triple negative breast cancer. In other specific embodiments, the breast cancer is ER+/HER2− breast cancer.

In one embodiment, provided herein is a method of treating cancer in a human patient comprising, wherein the patient has a tumor with a high mutational burden.

In specific embodiments, the cancer is selected from brain and spinal cancers. In particular embodiments, the brain and spinal cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET). In one embodiment, the brain or spinal cancer is a metastatic brain tumor or tumors.

In specific embodiments, the cancer is selected from cancers of the head and neck, including recurrent or metastatic head and neck squamous cell carcinoma (HNSCC), nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers. In specific embodiments, the skin cancer is unresectable or metastatic melanoma.

In specific embodiments, the cancer is selected from cancers of the reproductive system. In particular embodiments, the cancer is selected from the group consisting of breast cancers, cervical cancers, vaginal cancers, ovarian cancers, endometrial cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In some instances of these embodiments, the breast cancer is triple-negative breast cancer. In other instances, the breast cancer is ER+/HER2− breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gall bladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gall bladder adenocarcinomas, colorectal adenocarcinomas, microsatellite stable colorectal cancer, advanced microsatellite stable colorectal cancer, metastatic microsatellite stable colorectal cancer and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urothelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., classical Hodgkin refractory lymphoma), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In embodiments, the cancer is a metastatic tumor, for example, liver metastases from colorectal cancer or pancreatic cancer; and brain metastases from lung or breast cancer.

In embodiments, the cancer is selected from the group consisting of solid tumors and lymphomas. In particular embodiments, the cancer is selected from the group consisting of advanced or metastatic solid tumors and lymphomas. In more particular embodiments, the cancer is selected from the group consisting of malignant melanoma, head and neck squamous cell carcinoma, breast adenocarcinoma, and lymphomas. In aspects of such embodiments, the lymphomas are selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma.

In particular embodiments, the cancer is classified as stage III cancer or stage IV cancer. In some instances of these embodiments, the cancer is not surgically resectable.

### Compositions and Administration

When administered to a patient, a PNU Anthracycline Derivative can be administered as a component of a pharmaceutical composition that comprises a pharmaceutically acceptable excipient. Accordingly, in one embodiment, the present disclosure provides pharmaceutical compositions comprising an effective amount of a PNU Anthracycline Derivative, and one or more pharmaceutically acceptable carriers or excipients.

The PNU Anthracycline Derivatives are useful in preparing a medicament that is useful in treating a cellular proliferative disorder. In one embodiment, the PNU Anthracycline Derivatives are also useful for preparing a medicament that is useful in treating cancer.

In the pharmaceutical compositions and methods of the present disclosure, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms), and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Suitable lubricants include boric acid, sodium benzoate, sodium acetate, sodium chloride, and the like. Disintegrants include starch, methylcellulose, guar gum, and the like. Sweetening and flavoring agents, and preservatives may also be included where appropriate.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein. The molten homogeneous mixture is then poured into convenient sized molds, allowed to cool, and thereby solidify.

Additionally, the pharmaceutical compositions of the present disclosure may be formulated in sustained release form to provide the rate-controlled release of any one or more of the components or active ingredients to optimize therapeutic effects, i.e., anticancer activity and the like. Suitable dosage forms for sustained release include layered tablets containing layers of varying disintegration rates or controlled release polymeric matrices impregnated with the active components, and shaped in tablet form or capsules containing such impregnated or encapsulated porous polymeric matrices.

In one embodiment, the PNU Anthracycline Derivative is administered orally. In another embodiment, the PNU Anthracycline Derivative is administered orally in a capsule. In another embodiment, the PNU Anthracycline Derivative is administered orally in a tablet.

In another embodiment, the PNU Anthracycline Derivative is administered intravenously.

In another embodiment, the PNU Anthracycline Derivative is administered via subcutaneous injection.

In another embodiment, the PNU Anthracycline Derivative is administered via intertumoral injection.

In another embodiment, the PNU Anthracycline Derivative is administered topically. In a specific embodiment, the PNU Anthracycline Derivative is formulated as a cream that can be applied topically.

In still another embodiment, the PNU Anthracycline Derivative is administered sublingually.

In one embodiment, a pharmaceutical preparation comprising a PNU Anthracycline Derivative is in unit dosage form. In such form, the preparation is subdivided into unit doses containing effective amounts of the active components.

Compositions can be prepared using techniques such as conventional mixing, granulating or coating methods; and by using solid dispersion based upon the guidance provided herein. In one embodiment, the present compositions can contain from about 0.1% to about 99% of a PNU Anthracycline Derivative by weight or volume. In various embodiments, the present compositions can contain, in one embodiment, from about 1% to about 70%, or from about 5% to about 60%, or from about 10% to about 50% of a PNU Anthracycline Derivative by weight or volume.

In one embodiment, the present disclosure provides compositions comprising a PNU Anthracycline Derivative, a pharmaceutically acceptable carrier, and one or more additional therapeutic agents. In another embodiment, the present disclosure provides compositions comprising a PNU Anthracycline Derivative, a pharmaceutically acceptable carrier, and one additional therapeutic agent. In another embodiment, the present disclosure provides compositions comprising a PNU Anthracycline Derivative, a pharmaceutically acceptable carrier, and two additional therapeutic agents.

The quantity of a PNU Anthracycline Derivative in a unit dose of preparation may be varied or adjusted from about 1 mg to about 2500 mg. In various embodiments, the quantity is from about 10 mg to about 1000 mg, 1 mg to about 500 mg, 1 mg to about 100 mg, 1 mg to about 50 mg, 1 mg to about 20 mg, and 1 mg to about 10 mg.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., the Physicians' Desk Reference, 71′ Edition, 2017 (published by PDR Network, LLC at Montvale, NJ 07645-1725), presently accessible through www.pdr.net; the disclosures of which are incorporated herein by reference thereto.

If the patient is responding, or is stable, after completion of the therapy cycle, the therapy cycle can be repeated according to the judgment of the skilled clinician. Upon completion of multiple therapy cycles, the patient can be continued on the PNU Anthracycline Derivatives at the same dose that was administered in the treatment protocol. This maintenance dose can be continued until the patient progresses or can no longer tolerate the dose (in which case the dose can be reduced and the patient can be continued on the reduced dose).

The doses and dosage regimen of the additional therapeutic agent(s) used in the combination therapies described herein for the treatment of cellular proliferative disorders can be determined by the attending clinician, taking into consideration the approved doses and dosage regimen in the package insert; the age, sex and general health of the patient; and the type and severity of the cellular proliferative disorder. When administered in combination with one or more additional therapeutic agents, the PNU Anthracycline Derivative, and the additional therapeutic agent(s) can be administered simultaneously (i.e., in the same composition or in separate compositions one right after the other) or sequentially. This is particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms can therefore be advantageous.

The attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Generally, a total daily dosage of a PNU Anthracycline Derivative alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

For convenience, the total daily dosage may be divided and administered in portions during the day if desired. In one embodiment, the daily dosage is administered in one portion. In another embodiment, the total daily dosage is administered in two divided doses over a 24-hour period. In another embodiment, the total daily dosage is administered in three divided doses over a 24-hour period. In still another embodiment, the total daily dosage is administered in four divided doses over a 24-hour period.

The amount and frequency of administration of a PNU Anthracycline Derivative will be regulated according to the judgment of the attending clinician considering such factors as age, condition and size of the patient as well as severity of the symptoms being treated.

### Combination Therapy

In one aspect, the present methods for treating a cellular proliferative disorder can further comprise the administration of one or more additional therapeutic agents that are other than a PNU Anthracycline Derivative.

Accordingly, in one embodiment, the present disclosure provides methods for treating a cellular proliferative disorder in a patient, the method comprising administering to the patient: (i) a PNU Anthracycline Derivative, or a pharmaceutically acceptable salt thereof, and (ii) at least one additional therapeutic agent that is other than a PNU Anthracycline Derivative, wherein the amounts administered are together effective to treat a cellular proliferative disorder. In one embodiment, the cellular proliferative disorder treated is cancer.

When administering a combination therapy disclosed herein to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, the PNU Anthracycline Derivative, and an additional therapeutic agent may be present in fixed amounts (dosage amounts) in a single dosage unit (e.g., a capsule, a tablet and the like).

In one embodiment, the PNU Anthracycline Derivative is administered during a time when the additional therapeutic agent(s) exert their prophylactic or therapeutic effect, or vice versa.

In another embodiment, the PNU Anthracycline Derivative, and the additional therapeutic agent(s) are administered in doses commonly employed when such agents are used as monotherapy for treating cancer.

In another embodiment, the PNU Anthracycline Derivative, and the additional therapeutic agent(s) are administered in doses lower than the doses commonly employed when such agents are used as monotherapy for treating cancer.

In one embodiment, the PNU Anthracycline Derivative, and the additional therapeutic agent(s) are present in the same composition. In one embodiment, this composition is suitable for oral administration. In another embodiment, this composition is suitable for intravenous administration. In another embodiment, this composition is suitable for intertumoral administration. In another embodiment, this composition is suitable for subcutaneous administration. In still another embodiment, this composition is suitable for parenteral administration.

Cancers and proliferative disorders that can be treated or prevented using the combination therapy methods of the present disclosure include, but are not limited to, those listed above.

The PNU Anthracycline Derivative, and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy. Accordingly, in one embodiment, the PNU Anthracycline Derivative, and the additional therapeutic agent(s) act synergistically and are administered in doses lower than the doses commonly employed when such agents are used as monotherapy for treating cancer.

In one embodiment, the administration of the PNU Anthracycline Derivative, and the additional therapeutic agent(s) may inhibit the resistance of cancer to these agents.

The PNU Anthracycline Derivatives may be used in combination with one or more other active agents (collectively referred to herein as “additional therapeutic agents”), including but not limited to, other therapeutic agents that are used in the prevention, treatment, control, amelioration, or reduction of risk of a particular disease or condition (e.g., cancer). In one embodiment, a PNU Anthracycline Derivative is combined with one or more other therapeutic agents for use in the prevention, treatment, control amelioration, or reduction of risk of a particular disease or condition for which the PNU Anthracycline Derivatives are useful. Such other active agents may be administered, by a route and in an amount commonly used therefor, contemporaneously or sequentially with a compound of the present disclosure.

Combinations of the PNU Anthracycline Derivatives with one or more anticancer agents are within the scope of the present disclosure. Examples of such additional anticancer agents can be found in Cancer Principles and Practice of Oncology by V. T. Devita and S. Hellman (editors), 9^{th} edition (May 16, 2011), Lippincott Williams & Wilkins Publishers. A person of ordinary skill in the art would be able to discern which combinations of additional therapeutic agents would be useful based on the particular characteristics of the drugs and the cancer involved. Such additional therapeutic agents include the following: estrogen receptor modulators, programmed cell death protein 1 (PD-1) inhibitors, programmed death-ligand 1 (PD-L1) inhibitors, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors. HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints.

The additional therapeutic agents, and classes of additional therapeutic agents, disclosed below herein, are all useful in the combination therapies described herein.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

In the treatment of breast cancer (e.g., postmenopausal and premenopausal breast cancer, e.g., hormone-dependent breast cancer) the compound of formula (I) may be used with an effective amount of at least one antihormonal agent selected from the group consisting of: (a) aromatase inhibitors, (b) antiestrogens, and (c) LHRH analogues; and optionally an effective amount of at least one chemotherapeutic agent. Examples of aromatase inhibitors include but are not limited to: Anastrozole (e.g., Arimidex), Letrozole (e.g., Femara). Exemestane (Aromasin), Fadrozole and Formestane (e.g., Lentaron). Examples of antiestrogens include but are not limited to: Tamoxifen (e.g., Nolvadex), Fulvestrant (e.g., Faslodex), Raloxifene (e.g., Evista), and Acolbifene.

Examples of LHRH analogues include but are not limited to: goserelin (e.g., Zoladex) and leuprolide (e.g., leuprolide acetate, such as Lupron or Lupron Depot). Examples of additional therapeutic agents useful in the present compositions and methods include, but are not limited to, the following cancer chemotherapeutic agents: trastuzumab (e.g., Herceptin), gefitinib (e.g., Iressa), erlotinib (e.g., erlotinib HCl, such as Tarceva), bevacizumab (e.g., Avastin), cetuximab (e.g., Erbitux), and bortezomib (e.g., Velcade).

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylomithine, ILX23-7553. trans-N-(4′-hydroxyphenyl) retinamide, and N-4-carboxyphenyl retinamide.

“Cytotoxic/cytostatic agents” refers to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-┌diamine-platinum(II)┐bis┌diamine(chloro)platinum(II)┐tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, 4-demethoxy-3-deamino-3-aziridinyl-4-methylsulphonyl-daunorubicin (see WO 00/50032), Raf kinase inhibitors (such as Bay43-9006) and mTOR inhibitors (such as Wyeth's CCI-779).

An example of a hypoxia activatable compound is tirapazamine.

Examples of proteosome inhibitors include but are not limited to lactacystin and MLN-341 (Velcade).

Examples of microtubule inhibitors/microtubule-stabilizing agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, TDX258, the epothilones (see for example U.S. Pat. Nos. 6,284,781 and 6,288,237) and BMS188797. In an example the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane. 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5.5a,6,8,8a.9-hexohydrofuro(3′,4′:6.7)naphtho(2,3-d)-1,3-dioxol-6-one, 2,3-(methylenedioxy)-5-methyl-7-hydroxy-8-methoxybenzo[c]-phenanthridinium. 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in Publications WO03/039460, WO03/050064, WO03/050122, WO03/049527, WO03/049679, WO03/049678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an example inhibitors of mitotic kinesins include, but are not limited to inhibitors of KSP, inhibitors of MKLP1, inhibitors of CENP-E, inhibitors of MCAK and inhibitors of Rab6-KIFL.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24).5097-5116 (2003).

“Inhibitors of kinases involved in mitotic progression” include, but are not limited to, inhibitors of aurora kinase, inhibitors of Polo-like kinases (PLK: in particular inhibitors of PLK-1), inhibitors of bub-1 and inhibitors of bub-R¹. An example of an “aurora kinase inhibitor” is VX-680 (tozasertib).

“Antiproliferative agents” include antisense RNA and DNA oligonucleotides such as G3139, ODN698, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine. 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N₆-[4-deoxy-4-[N₂-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N₄-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. In one embodiment, a monoclonal antibody targeted therapeutic agent is Bexxar.

“HMG-CoA reductase inhibitor” refers to inhibitors of 3-hydroxy-3-methylglutaryl-CoA reductase. Examples of HMG-CoA reductase inhibitors that may be used include but are not limited to lovastatin, simvastatin, pravastatin, fluvastatin, atorvastatin, rosuvastatin and cerivastatin. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open-acid and lactone forms is included within the scope of the present disclosure.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). For an example of the role of a prenyl-protein transferase inhibitor on angiogenesis see European J of Cancer, Vol. 35, No. 9, pp. 1394-1401 (1999).

“Angiogenesis inhibitor” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib, steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists.

Other examples of angiogenesis inhibitors useful in the present combinations include, but are not limited to, endostatin, ukrain, ranpimase, IM862, 5-methoxy-4-[2-methyl-3-(3-methyl-2-butenyl)oxiranyl┐-1-oxaspiro┌2,5┐oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416), or a pharmaceutically acceptable salt thereof.

Additional therapeutic agents that modulate or inhibit angiogenesis and may also be used in combination with the PNU Anthracycline Derivatives, include agents that modulate or inhibit the coagulation and fibrinolysis systems (see review in Clin. Chem. La. Med. 38:679-692 (2000)). Examples of such agents include, but are not limited to, heparin, low molecular weight heparins and carboxypeptidase U inhibitors (also known as inhibitors of active thrombin activatable fibrinolysis inhibitor [TAFIa]).

Further examples of angiogenesis inhibitors include a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF.

“Agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the CHK1 and CHK2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refers to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001. Specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine. BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline. SU6668, STI571A, N-4-chlorophenyl-4-(4-pyridylmethyl)-1-phthalazinamine, and EMD121974, or a pharmaceutically acceptable salt thereof.

“Inhibitors of cell proliferation and survival signaling pathway” refers to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004/0102360, WO 03/086404. WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, U.S. Pat. Nos. 7,454,431, 7,589,068), inhibitors of Raf kinase (for example BAY-43-9006), inhibitors of MEK (for example CI-1040 and PD-098059), inhibitors of mTOR (for example Wyeth CCI-779), and inhibitors of PI3K (for example LY294002).

The present disclosure also encompasses combination therapies comprising NSAIDs which are selective COX-2 inhibitors. For purposes of the specification NSAIDs which are selective inhibitors of COX-2 are defined as those which possess a specificity for inhibiting COX-2 over COX-1 of at least 100-fold as measured by the ratio of IC₅₀ for COX-2 over IC₅₀ for COX-1 evaluated by cell or microsomal assays. Inhibitors of COX-2 that are useful in the present methods are: 3-phenyl-4-(4-(methylsulfonyl)phenyl)-2-(5H)-furanone; and 5-chloro-3-(4-methylsulfonyl)-phenyl-2-(2-methyl-5-pyridinyl)pyridine; or a pharmaceutically acceptable salt thereof. Compounds that have been described as specific inhibitors of COX-2 and are therefore also useful in the present disclosure include, but are not limited to, the following: rofecoxib, etoricoxib, parecoxib, BEXTRA® and CELEBREX® or a pharmaceutically acceptable salt thereof.

As used herein, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Combinations with additional therapeutic agents, other than anti-cancer agents, are also contemplated in the instant methods. For example, combinations of the PNU Anthracycline Derivatives with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. PPAR-γ agonists have been shown to inhibit the angiogenic response to VEGF in vitro; both troglitazone and rosiglitazone maleate inhibit the development of retinal neovascularization in mice (Arch. Ophthamol. 2001: 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. No. 60/235,708 and 60/244,697), or a pharmaceutically acceptable salt thereof.

Another embodiment of the present disclosure is the use of the PNU Anthracycline Derivatives in combination with gene therapy for the treatment of cancer. For an overview of genetic strategies to treating cancer see Hall et al., (Am. J. Hum. Genet. 61:785-789, 1997) and Kufe et al., (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998:5(8):1105-13), and interferon gamma (J. Immunol. 2000; 164:217-222).

The PNU Anthracycline Derivatives may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar), or a pharmaceutically acceptable salt thereof.

A PNU Anthracycline Derivative may also be administered with an immunologic-enhancing drug, such as levamisole, isoprinosine and Zadaxin, or a pharmaceutically acceptable salt thereof.

A PNU Anthracycline Derivative may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomvcin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir, or a pharmaceutically acceptable salt thereof.

A PNU Anthracycline Derivative may also be useful for treating or preventing cancer in combination with Pgp and/or BCRP inhibitors including: cyclosporin A, PSC833, GF120918, cremophorEL, fumitremorgin C, Ko132, Ko134, Iressa, Imatnib mesylate, EKI-785, C11033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC144-093, erythromycin, vincristine, digoxin and talinolol, or a pharmaceutically acceptable salt thereof.

A PNU Anthracycline Derivative may also be useful for treating or preventing cancer, including bone cancer, in combination with bisphosphonates, including but not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A PNU Anthracycline Derivative may also be useful for treating or preventing breast cancer in combination with aromatase inhibitors. Examples of aromatase inhibitors include but are not limited to: anastrozole, letrozole and exemestane, or a pharmaceutically acceptable salt thereof.

A PNU Anthracycline Derivative may also be useful for treating or preventing cancer in combination with siRNA therapeutics.

The PNU Anthracycline Derivatives may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553. U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139), or a pharmaceutically acceptable salt thereof.

In one embodiment, specific anticancer agents useful in the present combination therapies include, but are not limited to: pembrolizumab (Keytruda®), abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, NeosarX®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFSP); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elite®); Rituximab (Rituxan®); Ridaforolimus; sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®): thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); vorinostat (Zolinza®) and zoledronate (Zometa®), or a pharmaceutically acceptable salt thereof.

Thus, the scope of the present disclosure encompasses the use of the PNU Anthracycline Derivatives in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

Yet another example of the present disclosure is a method of treating cancer that comprises administering a therapeutically effective amount of a PNU Anthracycline Derivative in combination with paclitaxel or trastuzumab.

The therapeutic combination disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a PNU Anthracycline Derivative is combined with one or more other anti-cancer agents for use in the prevention, treatment, control amelioration, or reduction of risk of a particular disease or condition for which the PNU Anthracycline Derivatives are useful. Such other active agents may be administered, by a route and in an amount commonly used therefor, prior to, contemporaneously, or sequentially with a compound of the present disclosure.

The present disclosure also includes a pharmaceutical composition useful for treating or preventing cancer that comprises a therapeutically effective amount of a PNU Anthracycline Derivative and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

The present disclosure further relates to a method of treating cancer in a human patient comprising administration of a PNU Anthracycline Derivative and a PD-1 antagonist to the patient. The compound of the present disclosure and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is an anti-PD-1 antibody, independently selected from pembrolizumab, nivolumab, cemiplimab, sintilimab, tislelizumab, atezolizumab (MPDL3280A), camrelizumab and toripalimab. In other embodiments, the PD-L1 antagonist is an anti-PD-L1 antibody independently selected from atezolizumab, durvalumab and avelumab.

In one embodiment, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

When the PNU Anthracycline Derivatives are administered in combination with an anti-human PD-1 antibody (or antigen-binding fragment thereof), the anti-human PD-1 antibody (or antigen-binding fragment thereof) may be administered either simultaneously with, or before or after, the PNU Anthracycline Derivative. Either of the anti-human PD-1 antibody (or antigen-binding fragment thereof), and/or PNU Anthracycline Derivative of the present disclosure, or a pharmaceutically acceptable salt thereof, may be administered separately, by the same or different route of administration, or together in the same pharmaceutical composition as the other agent(s). The weight ratio of the anti-human PD-1 antibody (or antigen-binding fragment thereof) to PNU Anthracycline Derivative of the present disclosure, may be varied and will depend upon the therapeutically effective dose of each agent. Generally, a therapeutically effective dose of each will be used. Combinations including at least one anti-human PD-1 antibody (or antigen-binding fragment thereof), a PNU Anthracycline Derivative of the present disclosure, and optionally other active agents will generally include a therapeutically effective dose of each active agent. In such combinations, the anti-human PD-1 antibody (or antigen-binding fragment thereof), the PNU Anthracycline Derivative, and other active agents may be administered separately or in conjunction. In addition, the administration of one element may be prior to, concurrent with, or subsequent to the administration of other agent(s).

In one embodiment, this disclosure provides an anti-human PD-1 antibody (or antigen-binding fragment thereof), and/or PNU Anthracycline Derivative, and at least one other active agent as a combined preparation for simultaneous, separate or sequential use in treating cancer.

The disclosure also provides the use of a PNU Anthracycline Derivative of the present disclosure, for treating cancer, where the patient has previously (e.g., within 24-hours) been treated with an anti-human PD-1 antibody (or antigen-binding fragment thereof). The disclosure also provides the use of an anti-human PD-1 antibody (or antigen-binding fragment thereof) for treating a cellular proliferative disorder, where the patient has previously (e.g., within 24-hours) been treated with a PNU Anthracycline Derivative of the present disclosure.

The present disclosure further relates to methods of treating cancer, said method comprising administering to a subject in need thereof a combination therapy that comprises (a) a PNU Anthracycline Derivative of the present disclosure, and (b) an anti-human PD-1 antibody (or antigen-binding fragment thereof); wherein the anti-human PD-1 antibody (or antigen-binding fragment thereof) is administered once every 21 days.

Additionally, the present disclosure relates to methods of treating cancer, said method comprising administering to a subject in need thereof a combination therapy that comprises: (a) a PNU Anthracycline Derivative of the present disclosure, and (b) an anti-human PD-1 antibody (or antigen-binding fragment thereof. In specific embodiments, the cancer occurs as one or more solid tumors or lymphomas. In further specific embodiments, the cancer is selected from the group consisting of advanced or metastatic solid tumors and lymphomas. In still further specific embodiments, the cancer is selected from the group consisting of malignant melanoma, head and neck squamous cell carcinoma, MSI-H cancer, MMR deficient cancer, non-small cell lung cancer, urothelial carcinoma, gastric or gastroesophageal junction adenocarcinoma, breast adenocarcinoma, and lymphomas. In additional embodiments, the lymphoma is selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma. Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma. In particular embodiments, the cellular proliferative disorder is a cancer that has metastasized, for example, a liver metastases from colorectal cancer. In additional embodiments, the cellular proliferative disorder is a cancer is classified as stage III cancer or stage IV cancer. In instances of these embodiments, the cancer is not surgically resectable.

In embodiments of the methods disclosed herein, the anti-human PD-1 antibody (or antigen binding fragment thereof) is administered by intravenous infusion or subcutaneous injection.

In one embodiment, the present disclosure provides compositions comprising a PNU Anthracycline Derivative, a pharmaceutically acceptable carrier, and an anti-human PD-1 antibody (or antigen-binding fragment thereof).

In another embodiment, the present disclosure provides compositions comprising a PNU Anthracycline Derivative, a pharmaceutically acceptable carrier, and pembrolizumab.

In one embodiment, the present disclosure provides compositions comprising a PNU Anthracycline Derivative, a pharmaceutically acceptable carrier, and two additional therapeutic agents, one of which is an anti-human PD-1 antibody (or antigen-binding fragment thereof), and the other of which is independently selected from the group consisting of anticancer agents.

A compound of the present disclosure may be employed in conjunction with anti-emetic agents to treat nausea or emesis, including acute, delayed, late-phase, and anticipatory emesis, which may result from the use of a compound of the present disclosure, alone or with radiation therapy. For the prevention or treatment of emesis, a compound of the present disclosure may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another example, conjunctive therapy with an anti-emesis agent selected from a neurokinin-1 receptor antagonist, a 5HT3 receptor antagonist and a corticosteroid is disclosed for the treatment or prevention of emesis that may result upon administration of the PNU Anthracycline Derivatives.

A PNU Anthracycline Derivative may also be administered with an agent useful in the treatment of anemia. Such an anemia treatment agent is, for example, a continuous erythropoiesis receptor activator (such as epoetin alfa).

A PNU Anthracycline Derivative may also be administered with an agent useful in the treatment of neutropenia. Such a neutropenia treatment agent is, for example, a hematopoietic growth factor which regulates the production and function of neutrophils such as a human granulocyte colony stimulating factor. (G-CSF). Examples of a G-CSF include filgrastim.

The PNU Anthracycline Derivatives may be useful when co-administered with other treatment modalities, including but not limited to, radiation therapy, surgery, and gene therapy. Accordingly, in one embodiment, the methods of treating cancer described herein, unless stated otherwise, can optionally include the administration of an effective amount of radiation therapy. For radiation therapy, γ-radiation is preferred.

The methods of treating cancers described herein can optionally include the administration of an effective amount of radiation (i.e., the methods of treating cancers described herein optionally include the administration of radiation therapy).

The methods of treating cancer described herein include methods of treating cancer that comprise administering a therapeutically effective amount of a PNU Anthracycline Derivative in combination with radiation therapy and/or in combination with a second compound selected from: an estrogen receptor modulator, an androgen receptor modulator, a retinoid receptor modulator, a cytotoxic/ytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the additional therapeutic agents listed herein.

Additional embodiments of the present disclosure include the pharmaceutical compositions, combinations, uses and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### Kits

In one aspect, provided is a kit comprising a therapeutically effective amount of a PNU Anthracycline Derivative, or a pharmaceutically acceptable salt, solvate or ester of said compound and a pharmaceutically acceptable carrier, vehicle or diluent.

In another aspect provided is a kit comprising an amount of a PNU Anthracycline Derivative, and an amount of at least one additional therapeutic agent listed above, wherein the amounts of the two or more active ingredients result in a desired therapeutic effect. In one embodiment, the PNU Anthracycline Derivative, and the one or more additional therapeutic agents are provided in the same container. In one embodiment, the PNU Anthracycline Derivative, and the one or more additional therapeutic agents are provided in separate containers.

What is claimed:

1. A compound having a structural Formula (I): 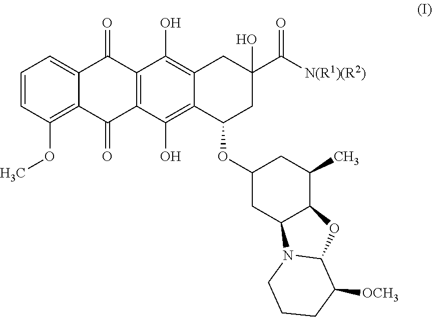
or a pharmaceutically acceptable salt thereof, wherein:
R¹ is H or C₁₋₆ alkyl;
R² is selected from C₁-C₆ hydroxyalkyl, 5 to 11-membered bridged bicyclic heterocycloalkyl, 5 to 11-membered fused bicyclic heterocycloalkyl, C₁-C₆ aminoalkyl, —(C₁-C₆ alkylene)-NR³C(O)—(C₁-C₆ aminoalkyl), —(C₁-C₆ alkylene)-NHC(O)NHNH₂, —(C₁-C₆ alkylene)-NR³, —(C₁-C₆ alkylene)-N(R³)—(C₁-C₆ alkylene)-N(R³)—NHC(O)—(C₁-C₆ aminoalkyl), —(C₁-C₆ haloalkylene)-NR³, —(C₁-C₆ alkylene)ₙ-(3 to 7-membered monocyclic heterocycloalkyl), —(C₁-C₆ alkylene)-NR³C(O)-(3 to 7-membered monocyclic heterocycloalkyl), —(C₁-C₆ alkylene)-NR³C(O)—(C₃-C₇ monocyclic cycloalkyl), —(C₁-C₆ alkylene)ₙ-(C₅-C₁₁ fused bicyclic cycloalkyl), —(C₁-C₆ alkylene)ₙ-(C₅-C₁₁ bridged bicyclic cycloalkyl), —(C₁-C₆ alkylene)ₙ-(C₃-C₇ monocyclic cycloalkyl), —(C₁-C₆ alkylene)ₙ-(C₆-C₁₀ aryl), —(C₁-C₆ alkylene)ₙ-(5 or 6-membered monocyclic heteroaryl), and 5-11 membered spirocyclic heterocycloalkyl, wherein said 3 to 7-membered monocyclic heterocycloalkyl group, said 5 to 10-membered fused bicyclic heterocycloalkyl group, said 5 to 10 membered bridged bicyclic heterocycloalkyl group, said C₃-C₇ monocyclic cycloalkyl group, said C₆-C₁₀ aryl group, said 5 or 6-membered monocyclic heteroaryl group, said C₅-C₁₁ bicyclic cycloalkyl group, and said 5-11 membered spirocyclic heterocycloalkyl group can each be optionally and independently substituted with one or more R^{A} groups, and wherein said 3 to 7-membered monocyclic heterocycloalkyl group can have one or more ring carbon atoms substituted with an oxo group;
alternatively, R¹ and R², taken together with the common nitrogen atom to which they are each attached, join to form: (i) a 3 to 7-membered monocyclic heterocycloalkyl group, (ii) a 5 to 11-membered bridged bicyclic heterocycloalkyl group, (iii) a 5 to 11-membered fused bicyclic heterocycloalkyl group, or (iv) a 5 to 11-membered spirocyclic heterocycloalkyl group, wherein said 3 to 7-membered monocyclic heterocycloalkyl group, said 5 to 11-membered bicyclic heterocycloalkyl group, said 5 to 11-membered fused heterocycloalkyl group, and said 5 to 11-membered spirocyclic heterocycloalkyl group can each be optionally and independently substituted with one or more R^{B} groups;
each occurrence of R³ is independently H or C₁₋₆ alkyl;
each occurrence of R^{A} is independently selected from C₁₋₆ alkyl, halo, —CN, —OR³, —(C₁-C₆ alkylene)ₙ-N(R³)₂, C₁-C₆ aminoalkyl, —(C₁-C₆ alkylene)-NHC(O)—(C₁-C₆ aminoalkyl), —O—(C₁₋₆ alkyl), —NHC(O)CH₂OH, and —(C₁-C₆ alkylene)-(3 to 7-membered monocyclic heterocycloalkyl), wherein a 3 to 7-membered monocyclic heterocycloalkyl group can be optionally and independently substituted with one or more R^{c} groups;
each occurrence of R^{B} is independently selected from C₁₋₆ alkyl, halo, —CN, —OR³, —N(R³)₂, C₁-C₆ aminoalkyl, —(C₁-C₆ alkylene)-NHC(O)—(C₁-C₆ aminoalkyl), —O—(C₁₋₆ alkyl), —NHC(O)CH₂OH, and —(C₁-C₆ alkylene)-(3 to 7-membered monocyclic heterocycloalkyl), wherein a 3 to 7-membered monocyclic heterocycloalkyl group can be optionally and independently substituted with one or more R^{c} groups;
each occurrence of R^{c} is independently selected from C₁₋₆ alkyl, halo, —CN, —OR³, —N(R³)₂, C₁-C₆ aminoalkyl, —O—(C₁₋₆ alkyl), —NHC(O)—(C₁-C₆ aminoalkyl), —(C₁-C₆ alkylene)-NHC(O)—(C₁-C₆ aminoalkyl), C₁₋₆ alkyl, halo, —CN, —OR³, —N(R³)₂, —O—(C₁₋₆ alkyl), and —NHC(O)CH₂OH; and
each occurrence of n is independently 0 or 1.

2. The compound of claim 1, or a pharmaceutically acceptable salt thereof, having the structural formula (Ia):

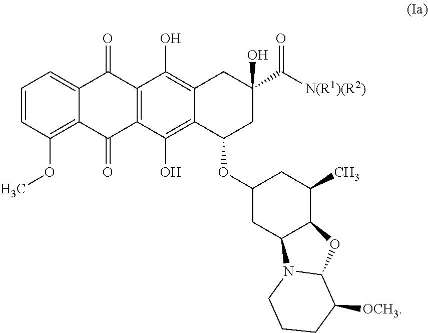

3. The compound of claim 1, or a pharmaceutically acceptable salt thereof, having the structural formula (Ib):

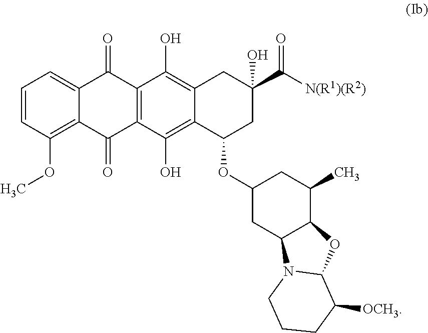

4. The compound of claim 1, or a pharmaceutically acceptable salt thereof wherein R¹ is H.

5. The compound of claim 4, or a pharmaceutically acceptable salt thereof, wherein R² is selected from C₃-C₇ monocyclic cycloalkyl, C₅-C₁₁ fused bicyclic cycloalkyl, C₅-C₁₁ bridged bicyclic cycloalkyl, C₆-C₁₀ aryl, 3 to 7-membered monocyclic heterocycloalkyl, 5 to 11-membered bridged bicyclic heterocycloalkyl, 5 to 11-membered fused bicyclic heterocycloalkyl, 5-11 membered spirocyclic heterocycloalkyl, and 5 or 6-membered monocyclic heteroaryl, wherein said C₃-C₇ monocyclic cycloalkyl group, said C₅-C₁₁ fused bicyclic cycloalkyl group, said C₅-C₁₁ bridged bicyclic cycloalkyl group, said C₆-C₁₀ aryl group, said 3 to 7-membered monocyclic heterocycloalkyl group, said 5 to 10-membered fused bicyclic heterocycloalkyl group, said 5 to 10 membered bridged bicyclic heterocycloalkyl group, said 5-11 membered spirocyclic heterocycloalkyl group, and said 5 or 6-membered heteroaryl group can each be optionally and independently substituted with one or more R^{A} groups.

6. The compound of claim 5, wherein R² is selected from:

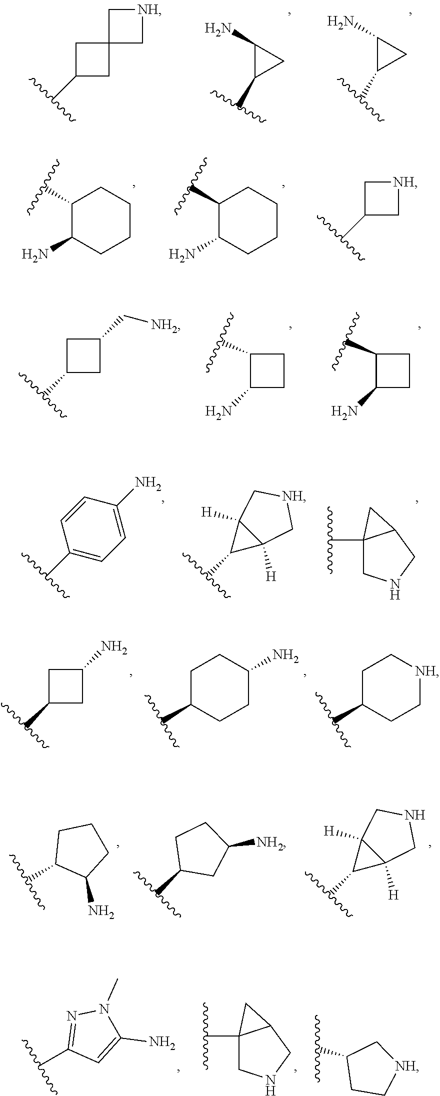

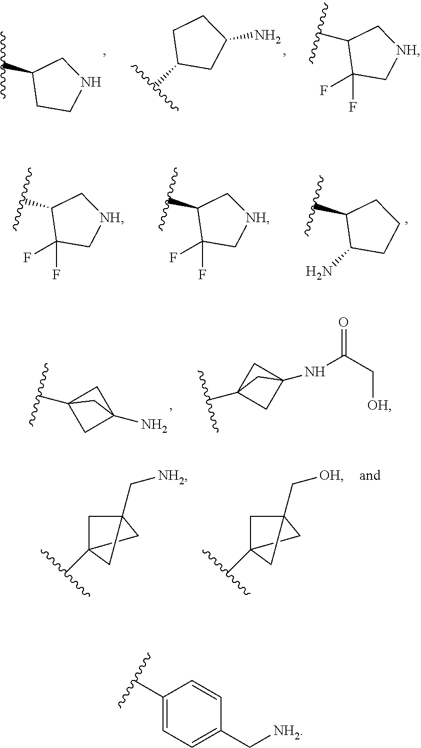

7. The compound of claim 4, or a pharmaceutically acceptable salt thereof, wherein R² is selected from C₁-C₆ hydroxyalkyl, C₁-C₆ aminoalkyl, —(C₁-C₆ alkylene)-NR³C(O)—(C₁-C₆ aminoalkyl), —(C₁-C₆ alkylene)-NHC(O)NHNH₂, —(C₁-C₆ alkylene)-N(R³)—(C₁-C₆ alkylene)-N(R³)—NHC(O)—(C₁-C₆ aminoalkyl), and —(C₁-C₆ haloalkylene)-NR³.

8. The compound of claim 7, wherein R² is selected from —CH₂CH₂NHC(O)CH₂NH₂, —CH₂CH₂NHC(O)NHNH₂, —CH₂CH₂NHC(O)CH(CH₃)NH₂, —(CH₂)₃NH(CH₂)₃NHC(O)CH₂NH₂, —CH₂CH₂N(CH₃)C(O)C(CH₃)(NH₂)CH(CH₃)₂, —CH₂CH₂NHCH₃, —(CH₂)₄NH₂, —CH₂C(CH₃)₂CH₂NH₂, —(CH₂)₃N(CH₃)CH₂CH₂CH₂NH₂, —CH₂C(CH₃)₂NH₂, —(CH₂)₃NH₂, —CH₂CH(NH₂)CF₃, —CH₂CH(NH₂)CHF₂, —CH₂CH₂CH(NH₂)CF₃, CH₂CH₂CH(NH₂)CHF₂, —(CH₂)₃NHCH₃, —CH₂CH₂OH, and —CH₂NH₂.

9. The compound of claim 4, or a pharmaceutically acceptable salt thereof, wherein R² is selected from —(C₁-C₆ alkylene)-(3 to 7-membered monocyclic heterocycloalkyl), —(C₁-C₆ alkylene)-NR³C(O)-(3 to 7-membered monocyclic heterocycloalkyl), —(C₁-C₆ alkylene)-NR³C(O)—(C₃-C₇ monocyclic cycloalkyl), —(C₁-C₆ alkylene)-(C₃-C₇ monocyclic cycloalkyl), —(C₁-C₆ alkylene)-(C₆-C₁₀ aryl), —(C₁-C₆ alkylene)-(C₅-C₁₁ fused bicyclic cycloalkyl group), and —(C₁-C₆ alkylene)-(5 or 6-membered monocyclic heteroaryl), wherein any of said 3 to 7-membered monocyclic heterocycloalkyl groups, any of said C₃-C₇ monocyclic cycloalkyl groups, said C₆-C₁₀ aryl group, said C₅-C₁₁ fused bicyclic cycloalkyl group, and said 5 or 6-membered monocyclic heteroaryl group can each be optionally and independently substituted with one or more R^{A} groups.

10. The compound of claim 9, wherein R² is selected from:

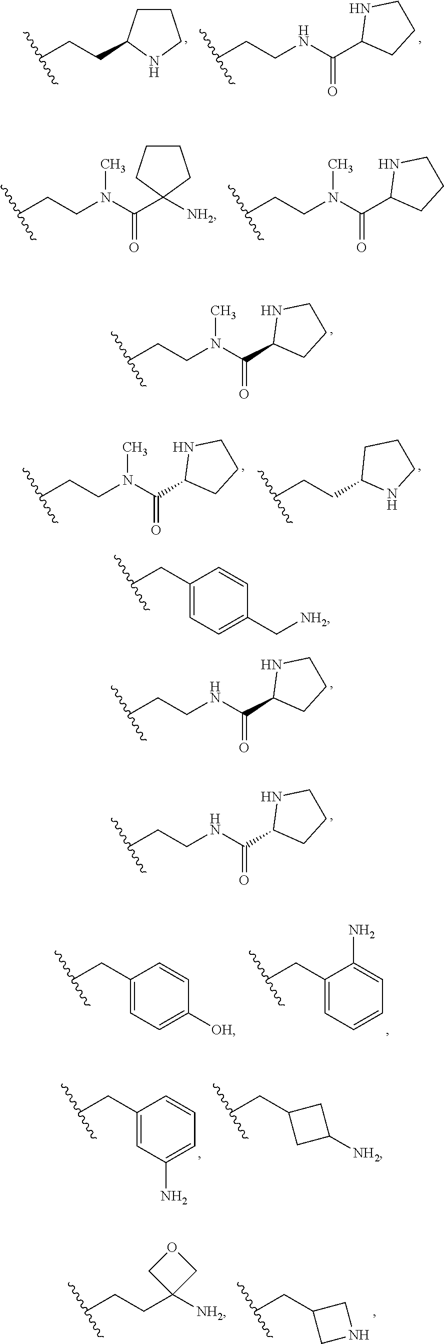

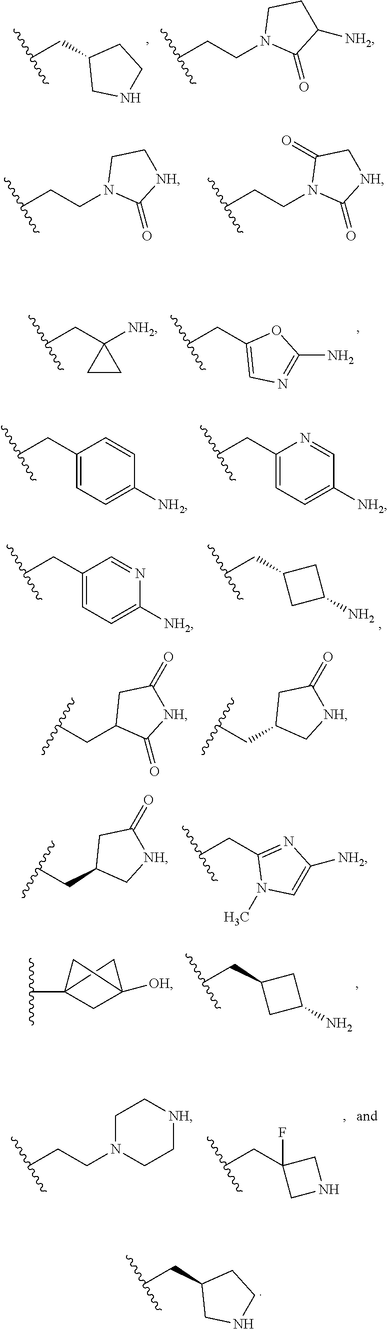

11. The compound of claim 1, or a pharmaceutically acceptable salt thereof wherein R¹ and R², taken together with the common nitrogen atom to which they are each attached, join to form a 3 to 7-membered monocyclic heterocycloalkyl group, 5 to 11-membered bridged bicyclic heterocycloalkyl group, or a 5 to 11-membered fused bicyclic heterocycloalkyl group, wherein said 3 to 7-membered monocyclic heterocycloalkyl group, said 5 to 11-membered bridged bicyclic heterocycloalkyl group, and said 5 to 11-membered fused bicyclic heterocycloalkyl group can each be optionally and independently substituted with one or more R^{B} groups.

12. The compound of claim 11, or a pharmaceutically acceptable salt thereof, wherein R¹ and R², taken together with the common nitrogen atom to which they are each attached, join to form a group selected from the following:

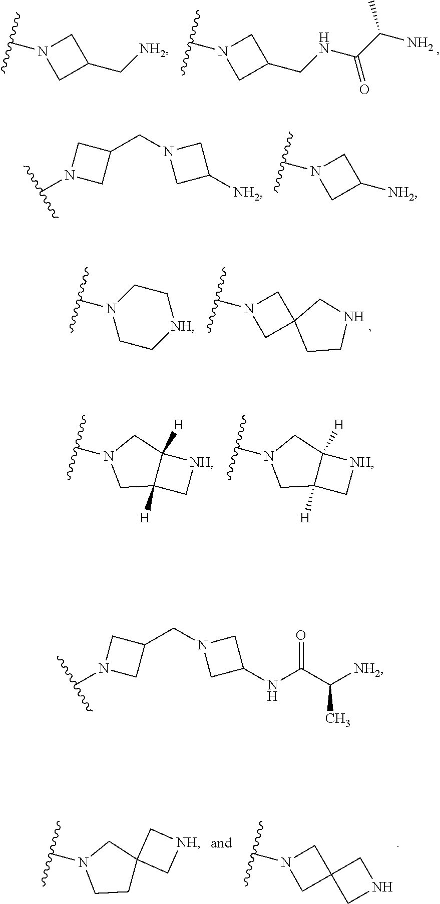

13. The compound of claim 1, or a pharmaceutically acceptable salt thereof, wherein
R¹ is H; and
R² is selected from —(C₁-C₁₀ alkylene)-(C₆-C₁₀ aryl), —(C₁-C₁₀ alkylene)-(3 to 7-membered monocyclic heterocycloalkyl), 5 to 10-membered bicyclic cycloalkyl, —(C₁-C₁₀ alkylene)-(C₃-C₇ monocyclic cycloalkyl); and 5 or 6-membered monocyclic heteroaryl, wherein said C₆-C₁₀ aryl group, said 3 to 7-membered monocyclic heterocycloalkyl group, said 5 to 10-membered bicyclic cycloalkyl group, said C₃-C₇ monocyclic cycloalkyl group; and said 5 or 6-membered monocyclic heteroaryl group can be optionally substituted by —NH₂.

14. The compound of claim 13, wherein R² is selected from:

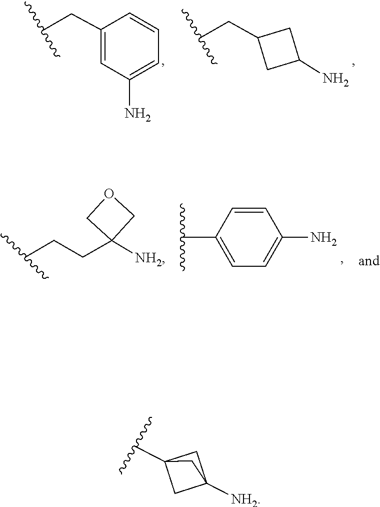

15. A compound, or a pharmaceutically acceptable salt thereof which is:

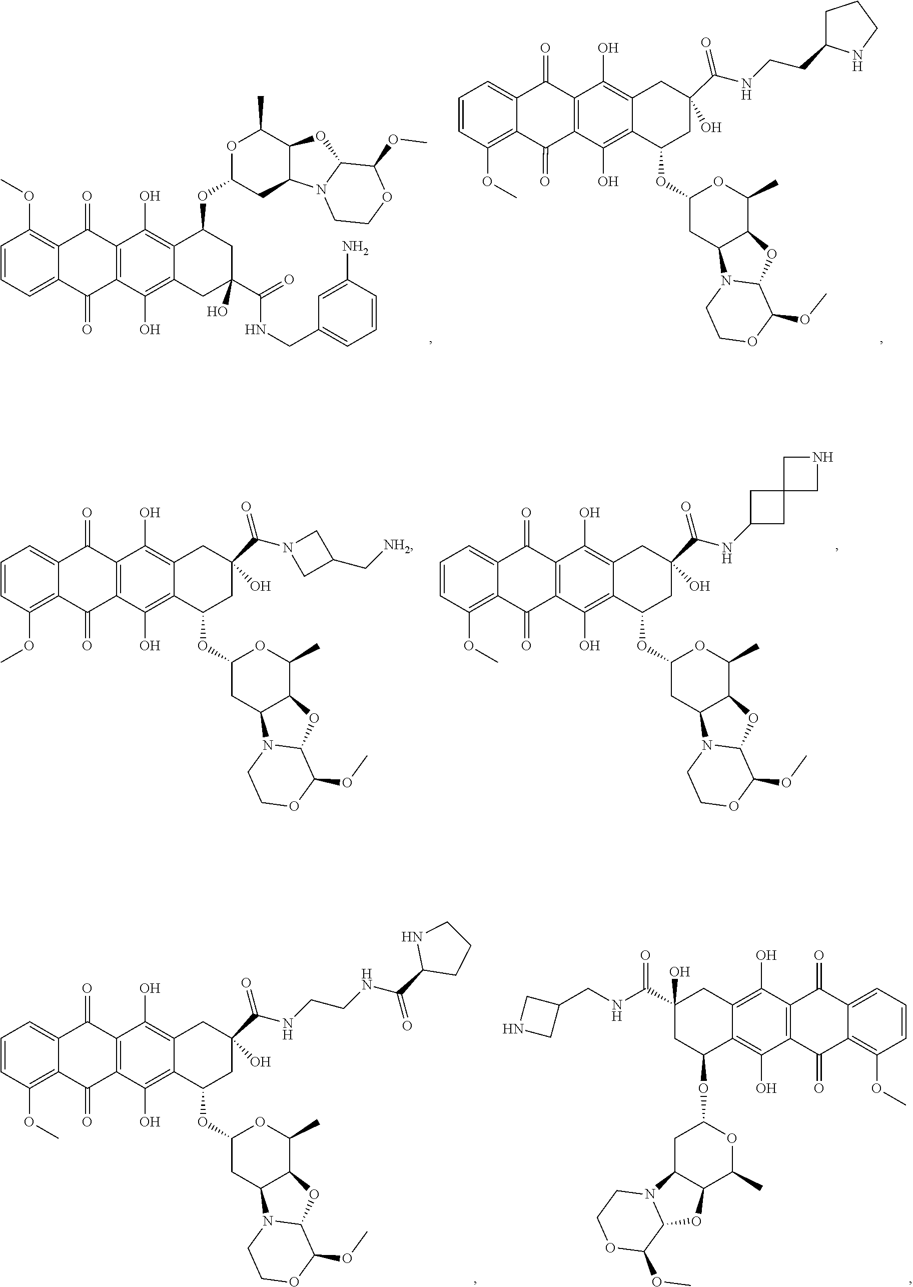

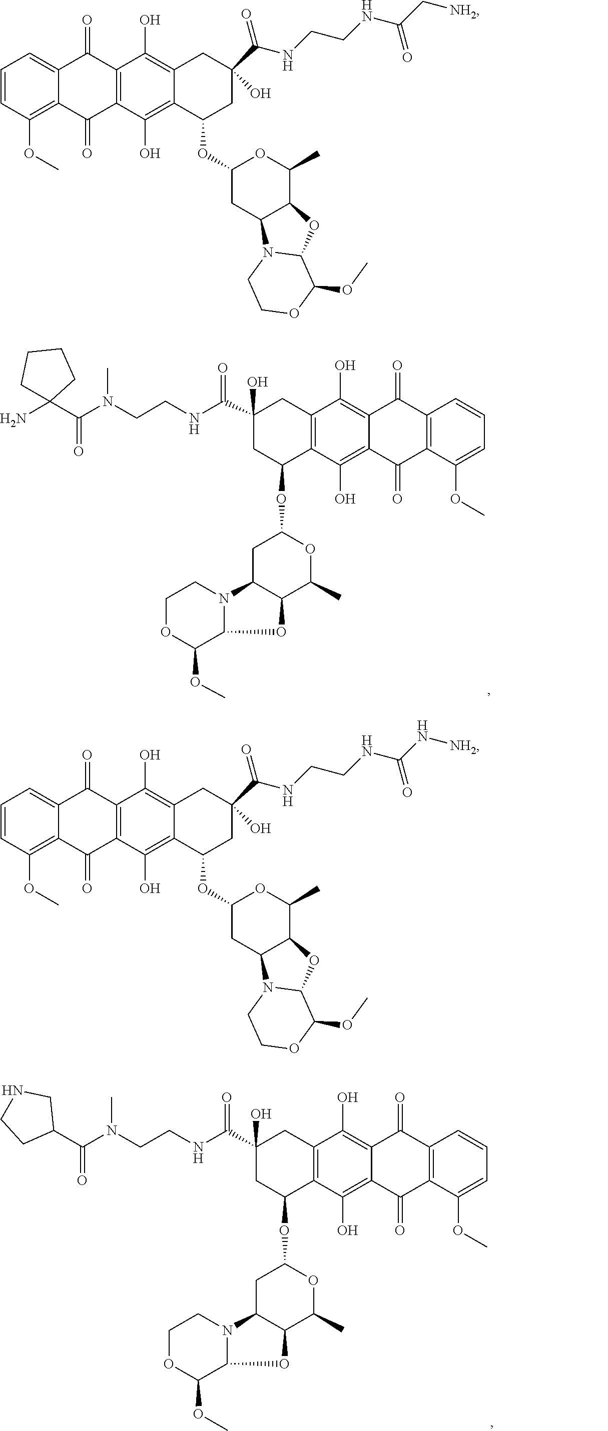

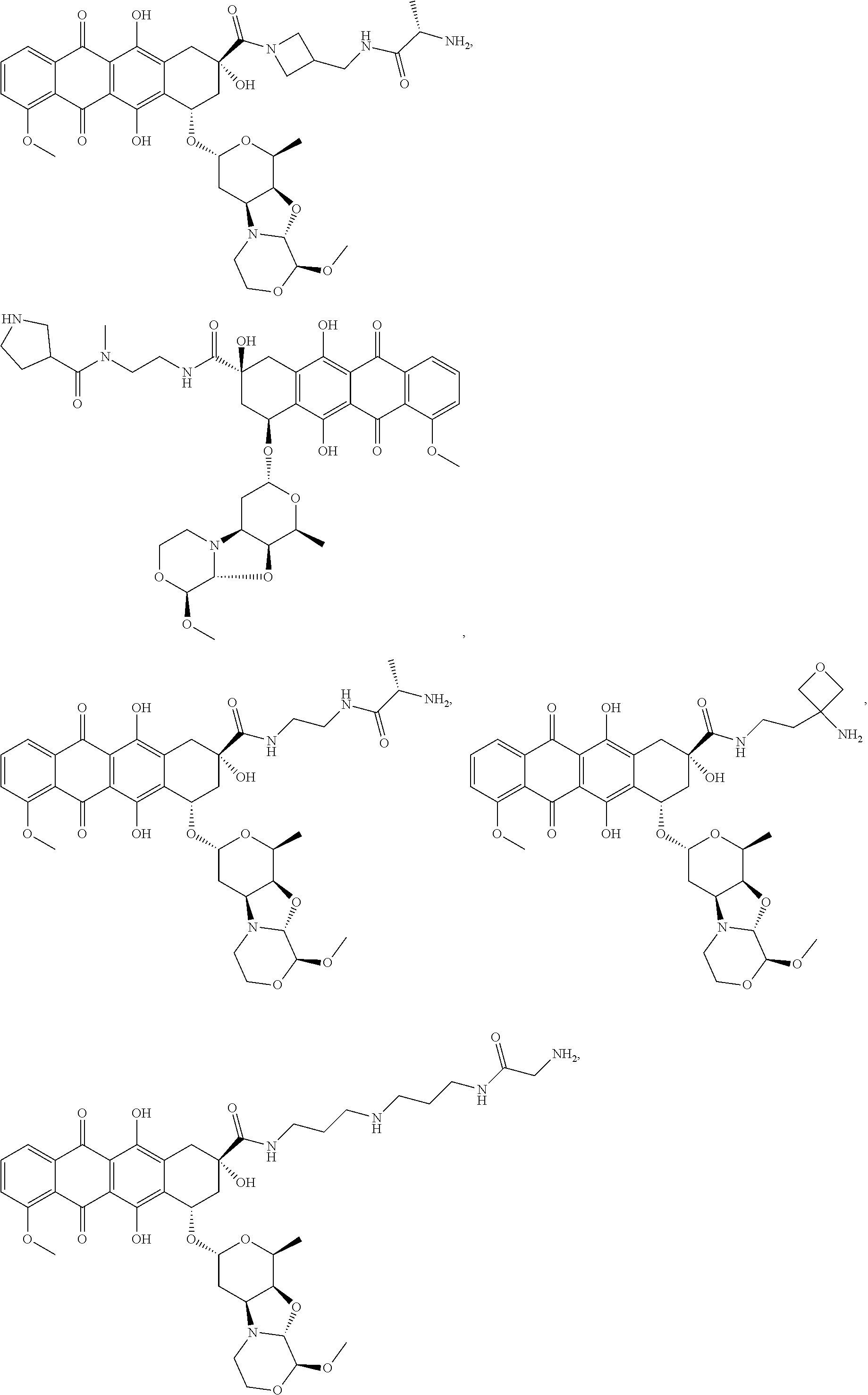

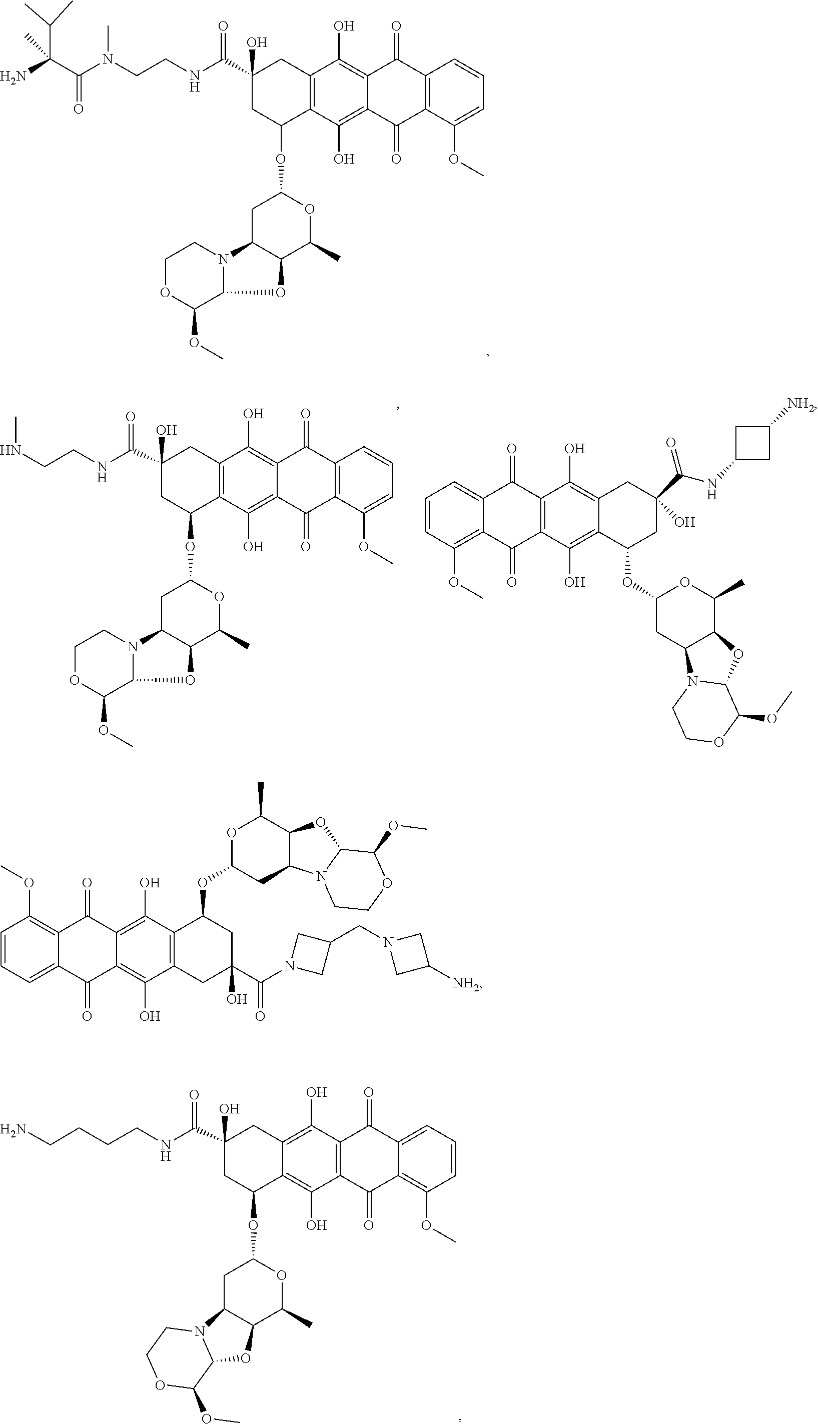

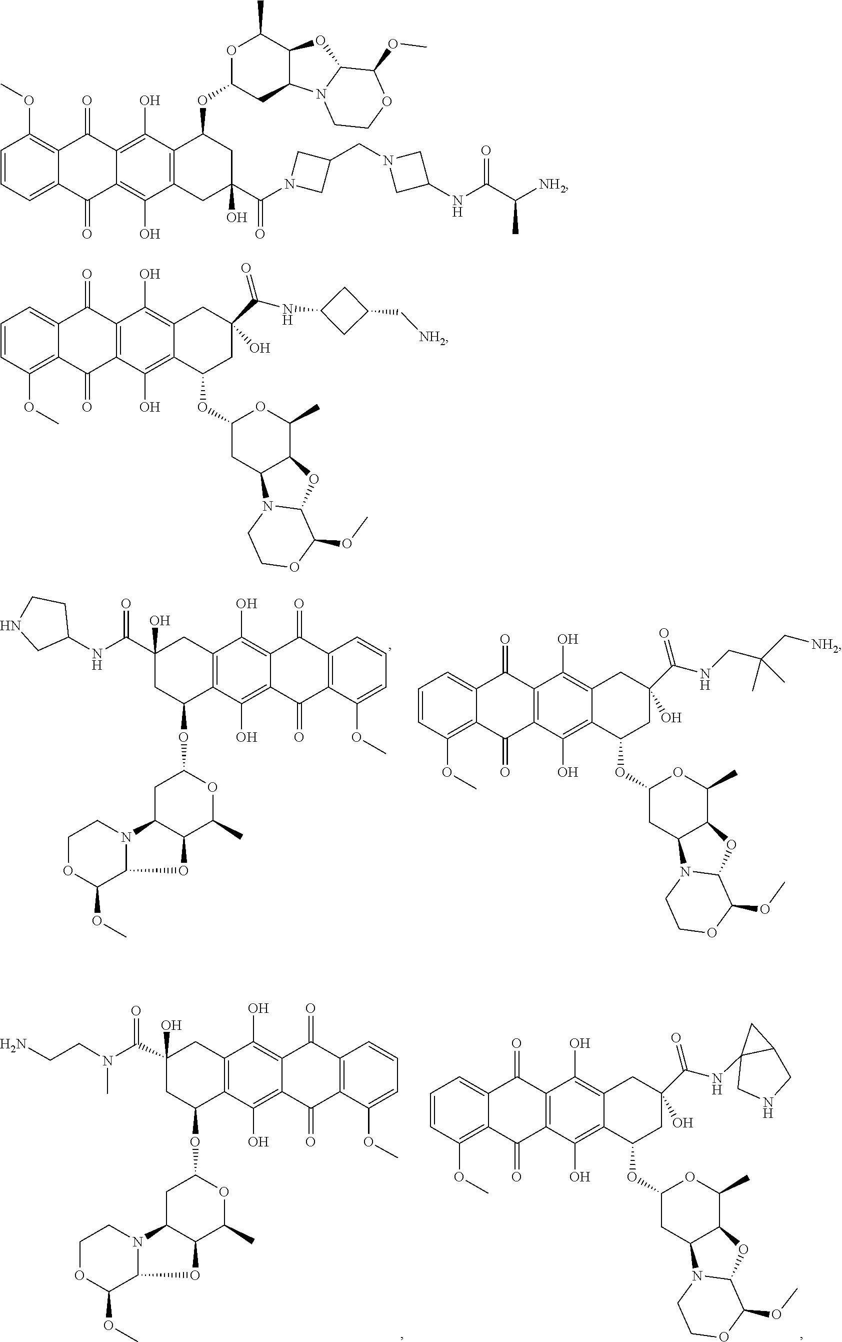

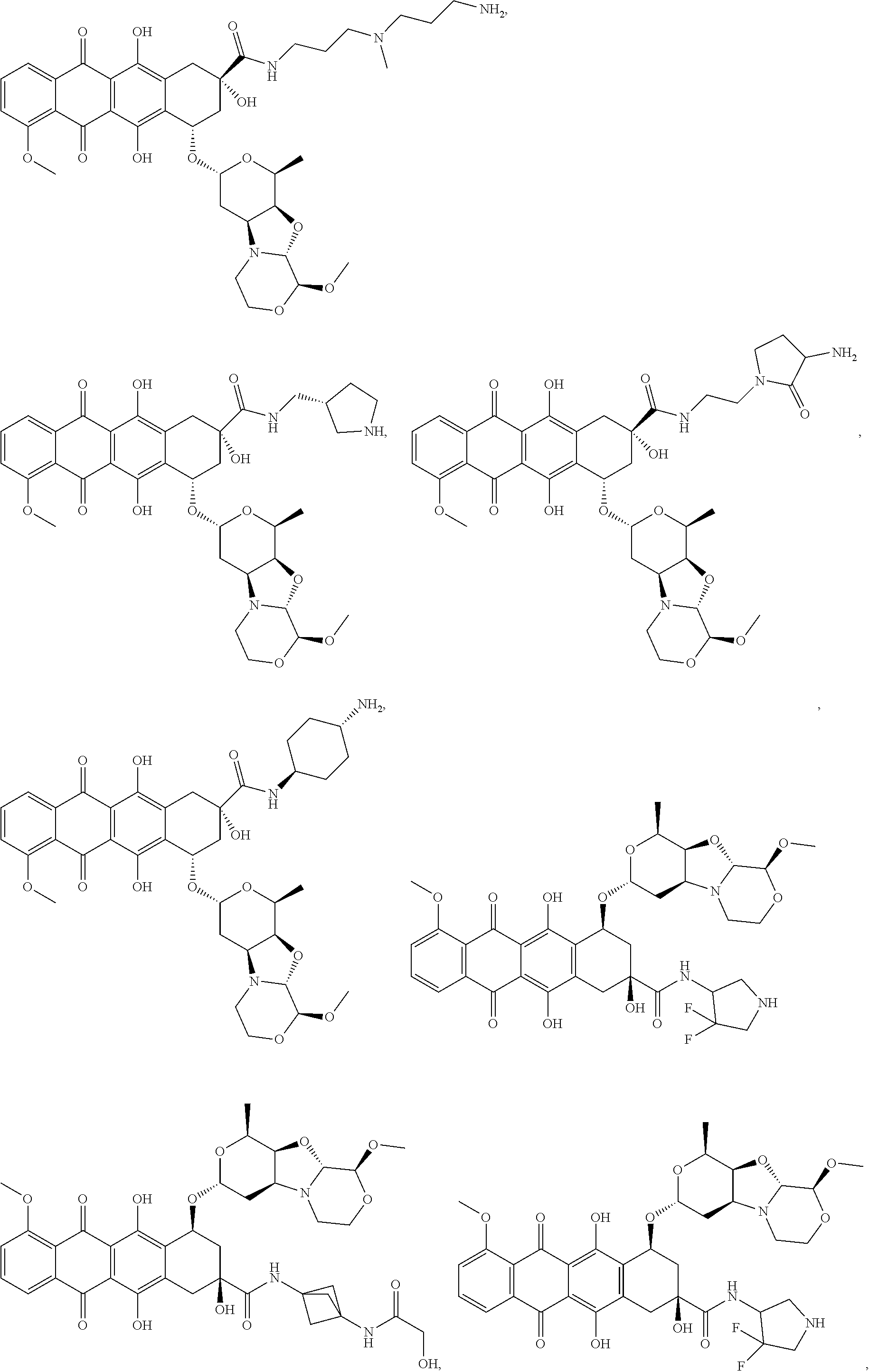

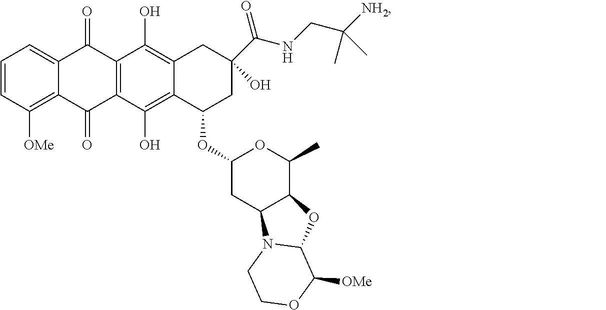 [SMILES: COc1cccc2c1C(=O)c1c(O)c3c(c(O)c1C2=O)C[C@@](O)(C(=O)NCC(C)(C)N)C[C@@H]3O[C@H]1C[C@H]2[C@H](O[C@@H]3[C@@H](OC)OCCN32)[C@H](C)O1]

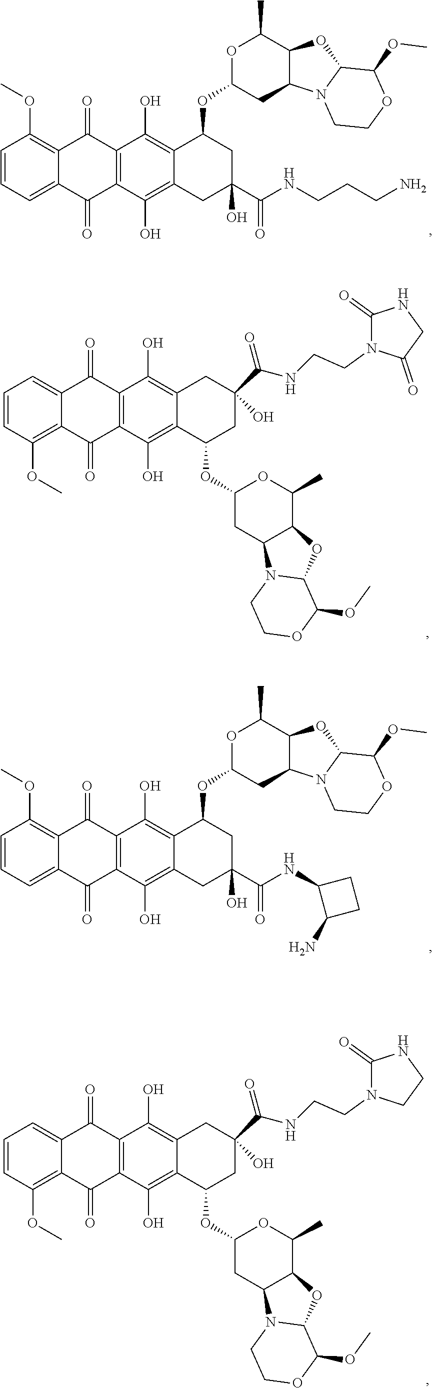

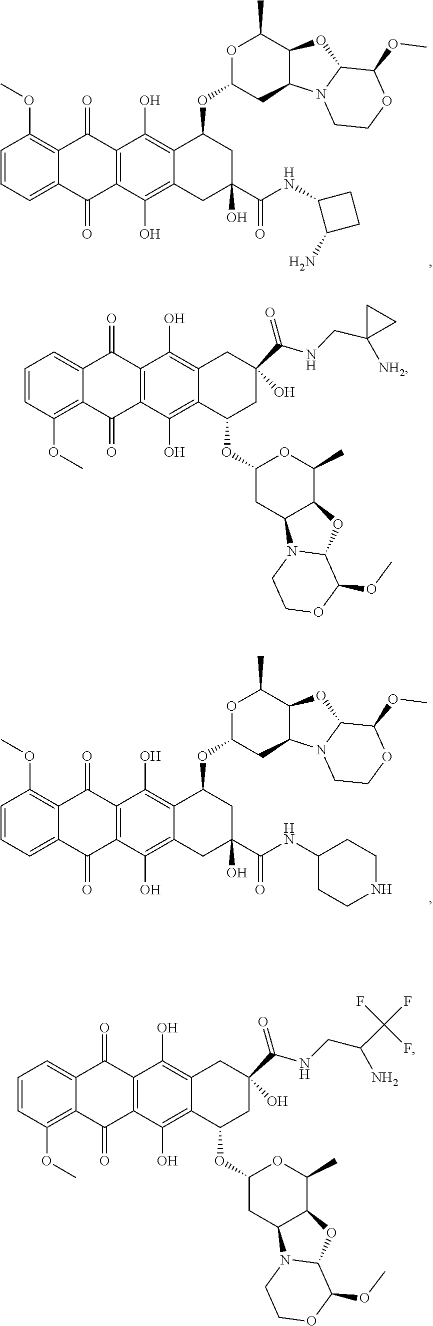

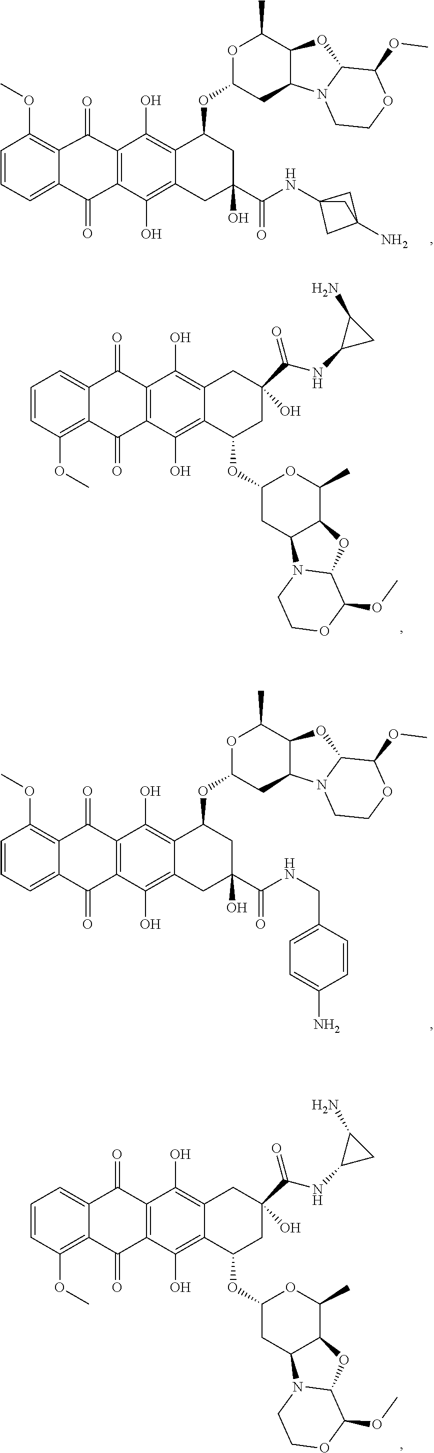

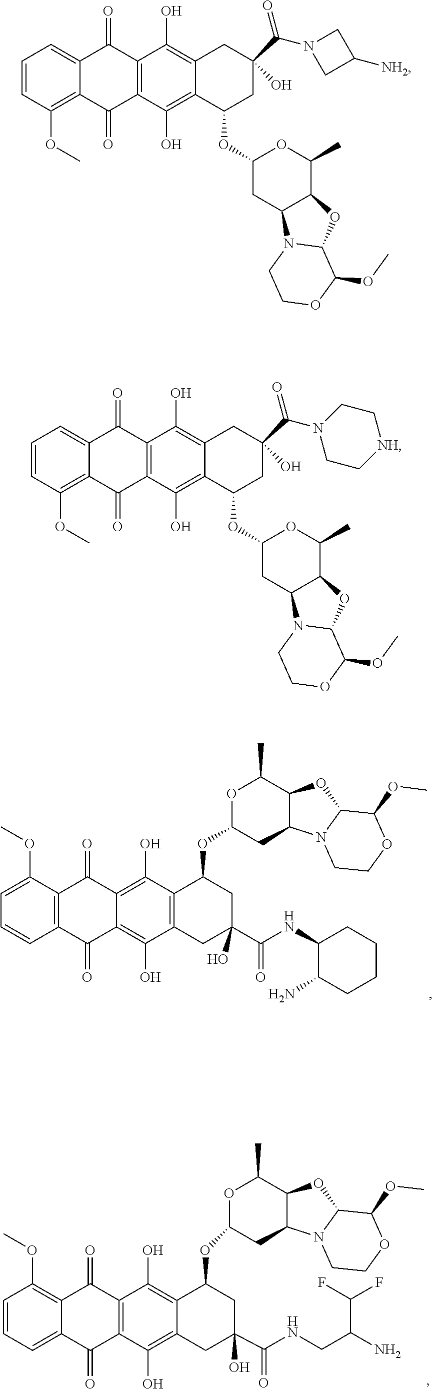

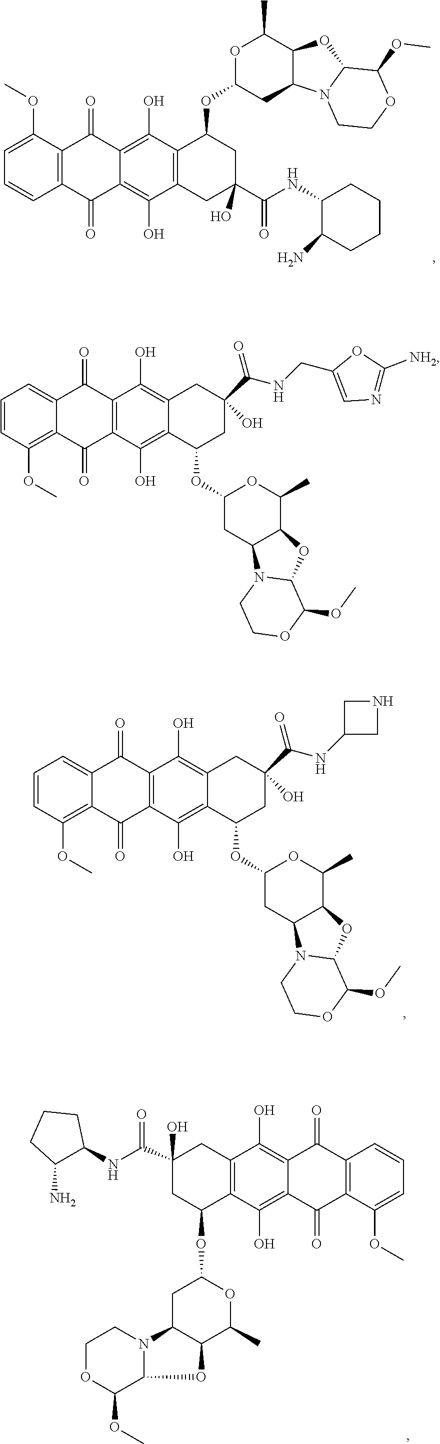

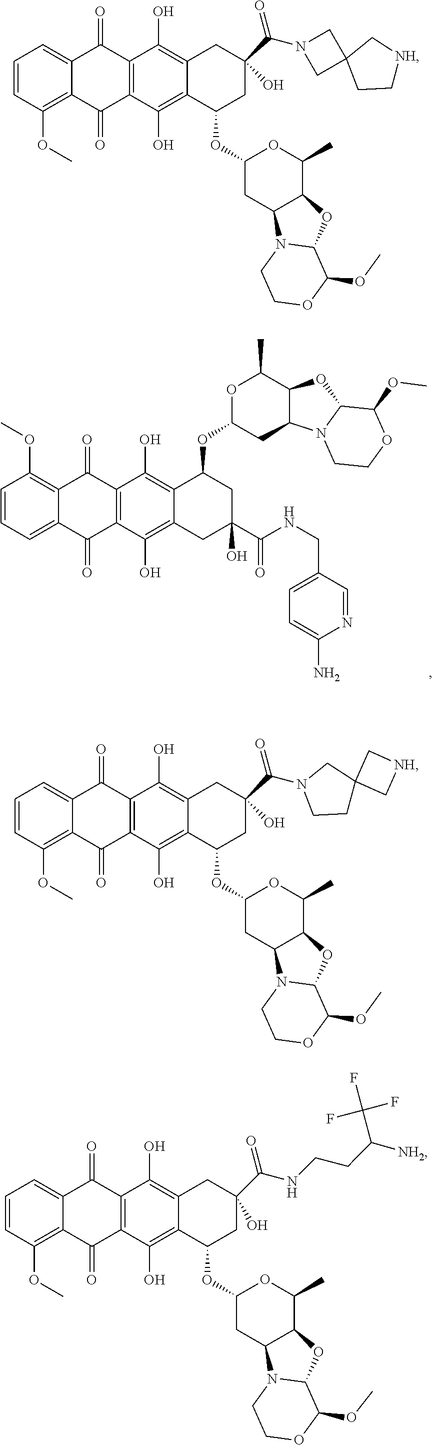

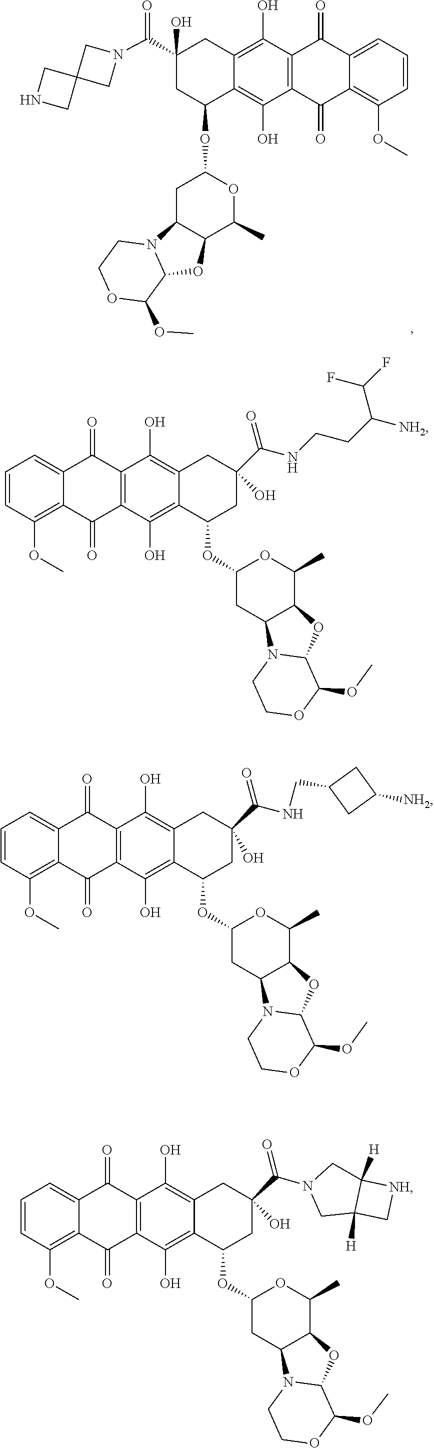

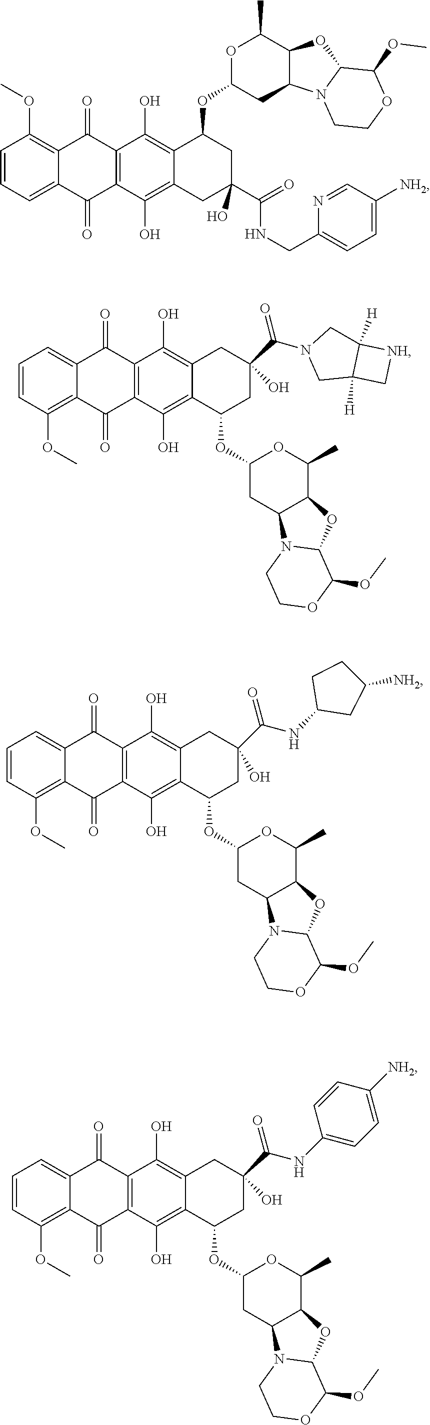

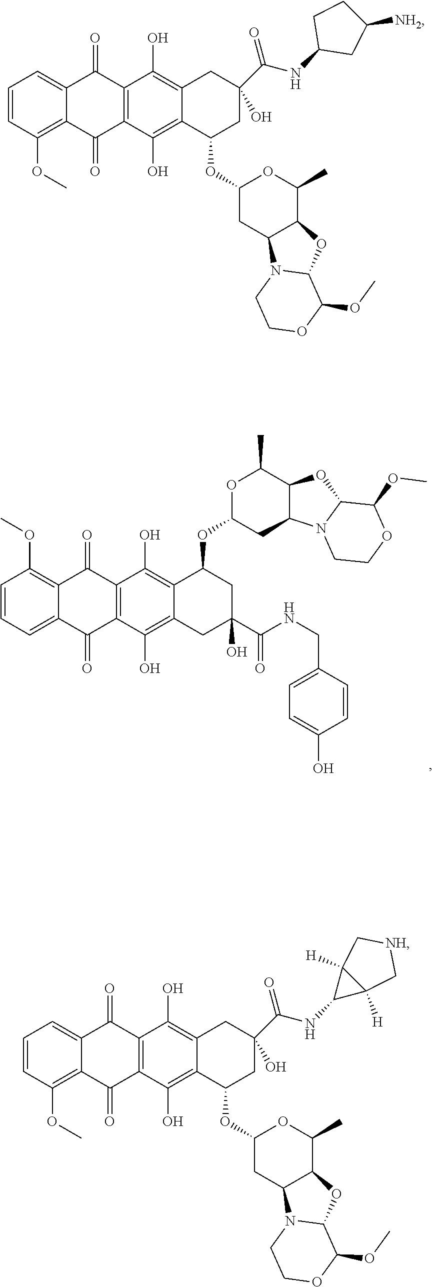

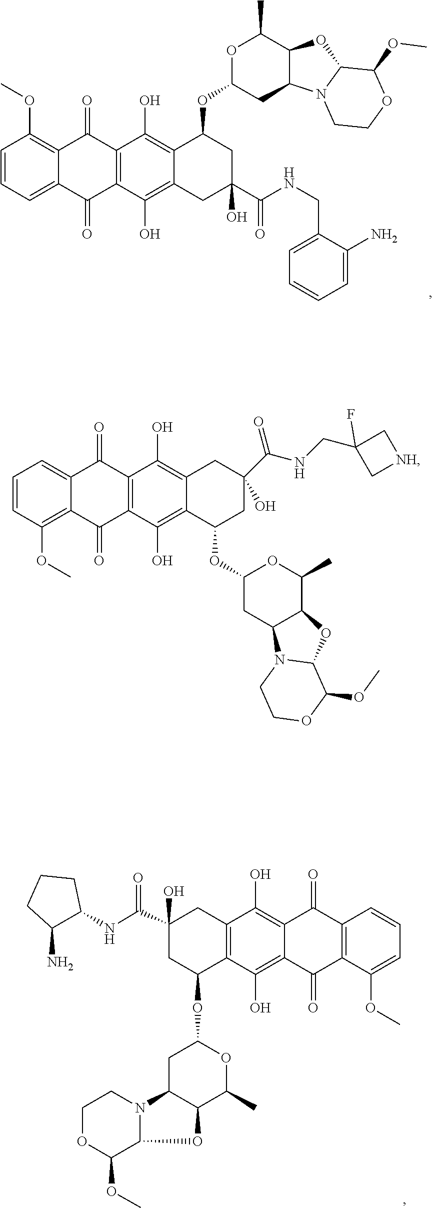

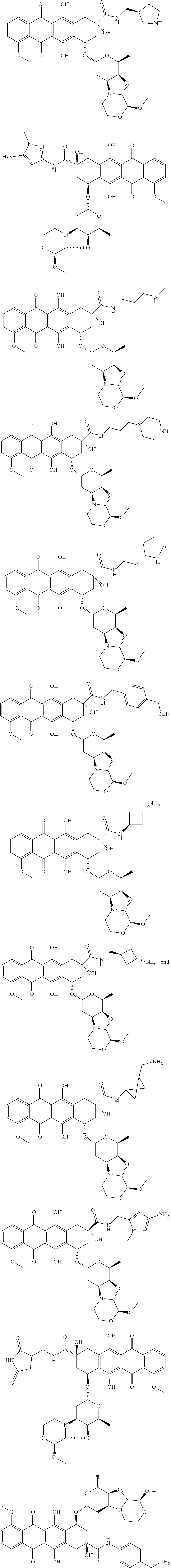

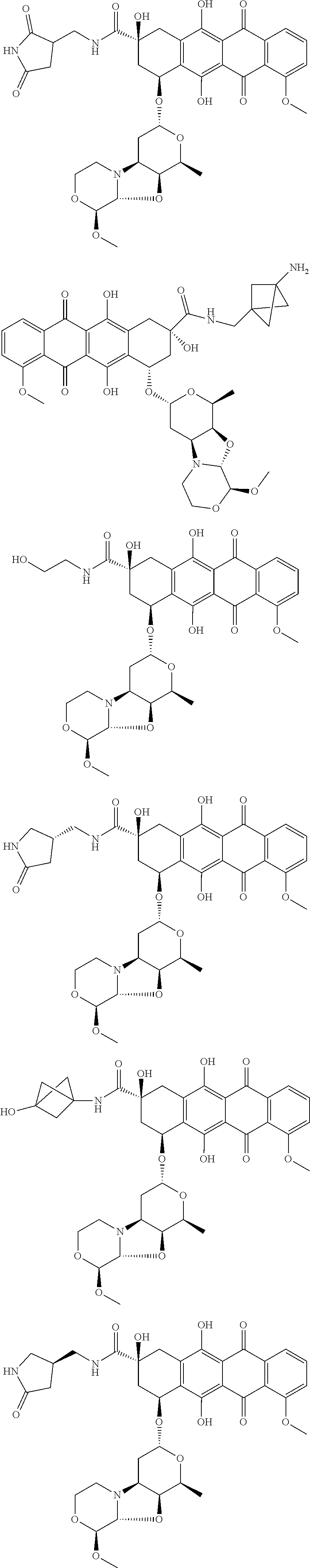

16. A pharmaceutical composition comprising an effective amount of the compound of claim 1, or a pharmaceutically acceptable salt thereof, and a pharmaceutically acceptable carrier.

17. The pharmaceutical composition of claim 16 further comprising one or more additional therapeutic agents, wherein said additional therapeutic agents are selected from anticancer agents.

18. A method of treating cancer in a patient in need thereof, comprising administering to the patient an effective amount of the compound of claim 1, or a pharmaceutically acceptable salt thereof.

19. The method of claim 18, further comprising administering one or more additional therapeutic agents, wherein said additional therapeutic agents are selected from anticancer agents.

20. (canceled)

21. The method of claim 19, wherein said additional therapeutic agents comprise pembrolizumab.

22. (canceled)
